(19) United States
(12) Patent Application Publication
(10) Pub. No.: US 2003/0220254 A1
(43) Pub. Date: Nov. 27, 2003

(54) Composition and method for preparation of an oral dual controlled release formulation of a protein and inhibitor

(75) Inventors: Mansoor A. Khan, Amarillo, TX (US); Vikas Agarwal, Plymouth, MN (US)

Correspondence Address: COX & SMITH INCORPORATED, SUITE 1800, 112 EAST PECAN STREET, SAN ANTONIO, TX, 782051536

(73) Assignee: TEXAS TECH UNIVERSITY SYSTEM

(21) Appl. No.: 10/405,968

(22) Filed: Mar. 31, 2003

Related U.S. Application Data
(60) Provisional application No. 60/368,489, filed on Mar. 29, 2002.

(51) Int. Cl. A61K 38/17; A61K 9/14

(52) U.S. Cl. 514/12; 424/486; 514/8

(57) ABSTRACT

The application discloses a composition and method for an oral dual controlled release formulation of a protein and absorption modifier. The coprecipitation technique for preparation of microcapsules of insulin as a model protein was evaluated and dissolution stability experiments in the presence of trypsin and α-chymotrypsin using chicken and duck ovomucoids as absorption modifiers were performed. The novel formulation improves the bioavailability of the protein with ovomucoids, while conserving the protein structure even after formulation and processing. An optimization design was used to evaluate critical process variables including the rate of addition of polymeric solution, compression pressure, and volume of water with respect to polymeric solution. The novel formulation incorporates controlled release characteristics of both protein and inhibitor to enhance protein stability and bioavailability with less potential for inhibitor concentration-related toxicity. The novel formulation utilizes an aqueous polymer having a pH sensitive solubility for targeted protein release.

This application claims the benefit under Title 35 United States Code §119(e) of U.S. Provisional Application No. 60/368,489 filed Mar. 29, 2002.

### BACKGROUND OF THE INVENTION

1. Field of Invention

The present invention relates, in general, to formulations for the oral delivery of proteins. More specifically, the present invention is directed to an oral dual controlled release formulation of a protein and inhibitor and to methods of preparing and using these compounds.

2. Description of the Related Art

The advent of recombinant DNA technology and proteomics has spawned tremendous interest and rapid development of research in therapeutic polypeptides and proteins. At this time, however, the common route of administration of these therapeutics is still through injections. In the past few decades, there has been great interest in the development of non-invasive routes for delivery of proteins and polypeptides. Among the non-invasive routes, the oral route for delivery of proteins is most desirable in terms of convenience and ease of administration. However, ingested proteins and polypeptides are generally broken down into amino acids by enzymes located in various regions of the gastrointestinal (GI) tract. These amino acids are then absorbed by the epithelium of the GI tract. Because the amino acids do not retain the original biological activity of the protein or polypeptide, their therapeutic efficacy is often lost.

Thus, the development of an oral delivery formulation for proteins faces several major obstacles. Oral bioavailability of proteins is extremely low due to extensive degradation in the gastrointestinal tract and low epithelial permeability. Further, the structure and conformation of proteins are easily altered when exposed to formulation and process conditions leading to a loss of biological activity.

Some examples of therapeutic proteins and polypeptides being aggressively studied for non-invasive route administration include calcitonin, growth hormone and insulin. Among these proteins, insulin (which is indicated for the treatment of insulin dependent diabetes mellitus (IDDM)), is the most widely studied protein for oral absorption.

Background

Insulin

Insulin was isolated from the bovine pancreas in the year 1922 (Banting & Best 1922) and has since been one of the most extensively studied molecules in biochemistry. It has the size of a polypeptide but all the structural features of a large protein. The elucidation of its primary structure (Sanger et al. 1955), chemical synthesis (Meienhofer & Schnabel 1965), crystallization in a variety of forms (Schlichtkrull 1958), the biosynthetic pathway in the pancreas (Steiner 1967), and the hormone's three dimensional structure (Adams et al. 1969; Baker et al. 2000) represented major scientific achievements and important milestones in our understanding of protein structure and function. Insulin was the first protein to be biosynthesized on a large scale using E-coli by recombinant DNA technology. Insulin was also the first molecule for which analogs were synthesized for therapeutic tailoring.

Insulin Structure

The primary structure of the human insulin molecule is shown in FIG. 1. Insulin contains 51 amino acid residues in two polypeptide chains (A and B) linked by two disulfide bonds. The A chain consists of 21 residues with an additional disulfide loop between A6 and A1 whereas the longer B chain holds 30 residues. The invariant residues (shown in black in FIG. 1) are responsible for the structural integrity and folding of the molecule. Non-polar residues such as cysteine constitute the hydrophobic core. The other residues are involved in the folding of the molecule into its three dimensional structure.

The secondary and tertiary structure of insulin is conserved among the species. The A chain forms two nearly antiparallel α-helices, A2 to A8 and A13 to A20. The B chain forms a single α-helix from B9 to B19 followed by a turn and a β-strand from B21 to B30.

The quaternary structure of insulin is related to its self-association. Insulin exists as a monomer only at low concentrations (<0.1 pM). At higher concentrations it dimerizes and in the presence of zinc ions, three dimers assemble at concentrations >0.01 mM further into a hexamer. At concentrations greater than 2 mM the hexamer is formed at neutral pH without the assistance of zinc ions (Hansen 1991). The various residues involved in self-association of insulin are indicated by their corresponding letters in FIG. 1. The two insulin molecules in the dimer are held together by vanderwaals and four hydrogen bonds (between B24 and B26 main chain residues) arranged as an anti-parallel (3-sheet structure between the two COOH-terminal strands of the B chain. The packing of the dimers around two zinc ions is associated with the burial of the remaining non-polar surface, but the interactions between the dimers in the hexamer are considerably weaker than those between monomers in the dimer.

Insulin is presumably stored in the granules of the pancreatic β-cells in the hexameric form with two molecules of Zn²⁺. Presence of zinc is believed to serve the functional role for the formation of crystals. Crystallization facilitates the conversion of proinsulin to insulin and storage of the hormone. During the absorption process, concentration of the hormone falls to physiological levels (nanomolar) which leads to the dissociation of hexamer to monomeric forms. Insulin exercises its action in the monomeric form.

Structure activity relationships of insulin reveal that a dozen invariant residues in A and B chains form a surface that interacts with the insulin receptor. These residues are Gly^{A1}, Glu^{A4}, GIn^{A5}, Tyr^{A19}, Asn^{A21}, Val^{B12}, Tyr^{B16}, Gly^{B23}, Phe^{B25}, Phe^{B25}, and Tyr^{B26}. These residues are also involved in dimer formation (De Meyts 1994) and are indicated by the letter D in FIG. 1.

Insulin circulates in blood as a free monomer, and its volume of distribution approximates the volume of extracellular fluid. Under fasting conditions the pancreas secretes about 40 micrograms of insulin per hour in the portal vein, to achieve a concentration of insulin in portal blood of 2 to 4 ng/mL (50 to 100 pU/mL) and peripheral circulation of 0.5 ng/mL (12 pU/mL). After ingestion of a meal, there is a rapid increase in the concentration of insulin in portal blood, followed by a similar and smaller rise in peripheral circulation. The goal of insulin therapy is to mimic this pattern. The half life of insulin in plasma for normal subjects is 5 to 6 minutes. Degradation of insulin occurs primarily in liver, kidneys, and muscle (Duckworth 1988). About 50% of the insulin that reaches the liver via the portal vein undergoes degradation and never reaches the systemic circulation.

Diabetes Mellitus and Physiological. Action of Insulin

Diabetes Mellitus is a common and serious disease characterized by hyperglycemia; altered metabolism of lipids; carbohydrates, and proteins, and an increased risk of complications from vascular disease. Acute hyperglycemia may cause life-threatening ketoacidosis. Chronic hyperglycemia is responsible for long-term side effects of diabetes such as retinopathy, nephropathy, neuropathy and cardiovascular symptoms. The hyperglycemia results from metabolic defect(s) causing a deficit in insulin secretion, insulin action, or both.

Previously, diabetes was classified along therapeutic lines, as either insulin-dependent (IDDM) or non-insulin dependent (NIDDM). However the American Diabetes Association recommends a more etiological classification. Diabetes resulting from deficiency of insulin secretion is classified as Type 1 diabetes, which commonly occurs in childhood. This has a relatively acute onset, requiring insulin for survival. Diabetes resulting from resistance to insulin (with or without concomitant insulin secretory defect) is classified as Type 2 diabetes. Type 2 diabetes usually occurs late in life, has a more insidious onset, and may or may not require an exogenous supply of insulin. Apart from these two categories, other forms of diabetes include cases arising from genetic defects of the beta-cell (maturity onset diabetes of the young genes, mitochondrial DNA mutations), genetic defects in insulin action, drug, chemical or disease induced pancreatic damage and endocrinopathies among others. The fourth general category comprises gestational diabetes mellitus, defined as any degree of glucose intolerance with onset or first recognition during pregnancy.

Diabetes is a chronic disease that has no cure. According to the official website of the American Diabetes Association (www.ada.org) there are 15.7 million people in the United States alone that have diabetes. It is the seventh leading cause of death. Many people first become aware that they have diabetes when they develop one of its life threatening complications such as blindness, kidney disease, nerve disease and heart disease. Diabetes is a costly health problem in America, with costs related to health care and lost productivity of over 100 billion annually.

Diabetes mellitus is characterized by decrease in circulating concentration of insulin (insulin deficiency) and decrease in response of peripheral tissues to insulin (insulin resistance). These abnormalities lead to alteration in the metabolism of carbohydrates, lipids, ketones, and amino acids. The central feature of the syndrome is hyperglycemia. The overview of insulin action is shown in FIG. 2. It has been hypothesized that the factor responsible for the development of most complications of diabetes is prolonged exposure of tissues to elevated concentrations of glucose (Pirart 1978). Insulin stimulates the storage of glucose in the liver as glycogen and in adipose tissue as triglycerides. It also stimulates the storage of amino acids in muscle as protein and promotes utilization of glucose in muscle for energy. These pathways, which are also enhanced by feeding, are indicated by the filled arrows in FIG. 2. Insulin inhibits the breakdown of triglycerides, glycogen, and protein and the conversion of amino acids to glucose (gluconeogenesis), as indicated by the open arrows (see FIG. 2). These pathways are increased during fasting and in diabetic states. The conversion of amino acids to glucose and of glucose to fatty acids occurs primarily in the liver.

Insulin Therapy

The primary objective of insulin therapy is to control glucose levels in blood. Insulin is used to achieve this objective in virtually all IDDM patients. Long term treatment of insulin relies on the subcutaneous injection of the hormone. The kinetics of subcutaneously delivered insulin differs from the physiological secretion of insulin in that it does not mimic the rapid rise and fall of insulin secretion in response to ingestion of nutrients, and insulin diffuses into the peripheral circulation instead of being released in the portal circulation. This causes the preferential effect of secreted insulin on hepatic metabolic processes to be eliminated. However, when treatment is performed carefully, considerable success has been achieved. Subcutaneous administration of insulin is the primary treatment for all patients with IDDM. In addition, insulin is critical for the management of diabetic ketoacidosis, and has an important role in the treatment of hyperglycemic, non-ketonic coma and in the perioperative management of both IDDM and NIDDM patients. The therapeutic goal is the normalization of blood glucose and all aspects of metabolism. Optimum treatment requires a coordinated approach to diet, exercise and administration of insulin. The goal of insulin therapy is to achieve a fasting blood glucose level concentration between 90 and 120 mg/dL and a 2 hour post-prandial value below 150 mg/dL. In less compliant patients or in those with defective responses of counterregulatory hormones, it may be necessary to accept higher fasting blood glucose concentrations like 140 mg/dL in fasting state and 200 mg/dL 12 hour postprandial.

Current Status on the Delivery of Insulin

Delivery of insulin has been a subject of intense scientific research worldwide for many decades. Advanced Drug Delivery Reviews (Vol 35, Nos 2,3 1999) recently devoted one complete issue to summarize various developments in insulin research. The pathogenesis of Type 1 diabetes is characterized by hyperglycemia (Graves & Eisenbarth 1999). The most common cause for this is the autoimmune destruction of the insulin producing cells of the pancreas. Role of genetic factors and environmental factors have been identified in the development of autoimmunity. The prediction of autoimmunity is possible by genetic screening before the development of auto-antibodies and immunological screening. Trials for the prevention of Type 1 diabetes can be primary (preautoimmunity), secondary (post-autoimmunity) or tertiary (post-diabetes). There are not many primary prevention trials to discuss due to the incomplete understanding of the development of autoimmunity. Trials planned include removal of potential diabetogenic exposures in early childhood. Secondary prevention trials are initiated after signs of autoimmunity have been detected. Treatment agents include cyclosporine, insulin and nicotinamide. There are risks associated with both primary and secondary prevention methods for Type 1 diabetes.

Pathogenesis of NIDDM is characterized by abnormal blood glucose homeostasis, resulting in hyperglycemia (Jun et al. 1999). The pathogenesis of Type 2 diabetes is affected profoundly by genetic and environmental factors. It is suggested that the genetic component plays an important etiological role in the development of NIDDM. Patients with a genetic predisposition undergo a slow transition from a normal state to hyperglycemia due to a combination of insulin resistance and defects in insulin secretion. Although candidate genes responsible for insulin resistance and defeats in insulin secretion are reported, a specific gene has not been identified. Examples of candidate genes for secretory defects include insulin gene glucose transporter (GLUT)-2 gene and for insulin resistance include insulin receptor gene (GLUT)-4, (GLUT)-1. Environmental factors that affect the pathogenesis include obesity, physical activity, and age.

Current Status and Prospects of Future Parenteral Delivery Regimens, Strategies and Delivery Systems for Diabetes Treatment (Jeandidier & Boivin 1999)

The normal route of administration of insulin by injection is through the subcutaneous route. Current parenteral delivery regimens also include administration of insulin by insulin pens, insulin injectors, and continuous subcutaneous infusion. All of these approaches suffer from limitations with respect to adequate control of blood glucose levels. Approaches to improve these limitations involve the use of insulin analogs and administration of peptides such as amylin, glucagon-like peptide, insulin growth factor 1 and C-peptide. Other routes of administration involve the use of implantable insulin infusion devices. The ulimate goal remains the development of an automated, glucose-controlled device with extended duration of action.

Intranasal Insulin Delivery and Therapy (Hinchcliffe & Illum 1999)

Intranasal delivery of insulin has been extensively investigated as an alternative to subcutaneous injection for the treatment of diabetes. The pharmacokinetic profile of intranasal insulin closely mimics the “pulsatile” pattern of insulin secretion during meal times. This suggests that intranasal delivery insulin has considerable potential for controlling postprandial hyperglycemia. The bioavailability of insulin administered by the intranasal route is minimal. To improve the bioavailability, absorption enhancers have been incorporated.

Inhaled Insulin (Patton et al. 1999)

Regular insulin can be administered by the pulmonary route for mealtime glucose control in diabetics. Absorption of insulin is possible without the use of penentration enhancers. Questions about variability in dosing have been addressed by the design of new reproducible delivery systems. Controlled studies in humans for three months indicated that inhaled insulin provides equivalent glucose control when directly compared with subcutaneous injection. For Type 2 diabetes patients, adjunct therapy with inhaled insulin markedly improved glycemic control with low risk of hypoglycemia. The major advantage of delivery of insulin by the pulmonary route is earlier peak time (5-60 minutes when compared to 6-150 minutes for the subcutaneous route).

Treatment of Type 1 Diabetes Using Encapsulated Islets (Soon-Shiong 1999)

Since Type 1 diabetes is associated with destruction of pancreatic islets that secrete insulin, transplantation of islets has been pursued as an alternative approach. Transplantation is associated with the use of high-dose immunosuppressive drugs. This approach has two serious limitations: rejection of transplantation and potentially serious side-effects of the drugs. Encapsulation circumvents the need for immunosuppression because the transplanted living cells are surrounded by a semi-permeable membrane which protects them from the host's immune system. An example of a recent encapsulated islet system includes alginate-polylysine spherical-bead microcapsules that have the required large surface area, enhanced nutrition and oxygen supply, precisely tailored porosity, maximum protection from membrane failure and direct injectability into the peritoneal cavity. This formulation was tested in a 38-year-old white male with insulin-dependent diabetes for 30 years. Encapsulated islets (10,000/kg) were injected directly into the peritoneal cavity through a 2 cm midline incision. Insulin secretion from the transplanted cells was detected within 24 hours after injection, and continued for more than 58 months. The patient reported significant improvement in the quality of life including a decrease in his lower extremity peripheral neuropathy symptoms, in increased energy level, an ability to walk further, a general feeling of improved health, and no adverse effects. Encapsulated islet transplantation appears to hold promise for the treatment of diabetes in the future. The main limitations are: (1) finding microcapsules with in vivo biocompatibility and increased mechanical stability and (2) finding sufficient sources of insulin-producing tissue.

Biohybrid Artificial Pancreas Based on Macrocapsule Device (Hou & Bae 1999)

Refillable biohybrid artificial pancreases (BAP) are proposed as an alternative to single use BAP. The design features include: (1) use of a thermally reversible synthetic hydrogel made of N-isopropylacrylamide-based copolymer as an extracellular matrix that facilitates recharging of encapsulated islets, (2) the fabrication of BAP device involves a pouch system composed of an inert processable immunoprotective membrane with appropriate physical, chemical and transportation properties, (3) the viability and function of the islets is maintained by the use of an oxygen-carrying polymer, and (4) stimulation of insulin secretion by incorporation of biospecific polymers within the matrix that also decreases the number of islets required.

Site-Specific Insulin Conjugates with Enhanced Stability and Extended Action Profile (Uchio et al. 1999)

Two different types of insulin conjugates were synthesized to develop an alternative to subcutaneous injection of insulin suspension to maintain basal levels. These conjugates were glycosylated insulins and PEG-insulin conjugates. Types of insulin included galactosylated, mannosylated and fucosylated forms. PEG-insulin conjugates were prepared by using carboxyl derivatives of methoxypolyethylene glycols with different molecular weights. Immonogenicity of mono-substituted glycosylated insulins was comparable to that of native insulin while disubstituted glycosylated insulins demonstrated elevated immune response in vivo. Immonogenicity of pegylated insulin decreased as the molecular weight of PEG was increased. Among the glycosylated insulin, only PheB 1 demonstrated bioactivity, immunogenic properties, and stability features. After subcutaneous administration, PheB 1 insulin demonstrated an action profile of intermediate-acting insulin preparations. Preliminary pharmacodynamic experiments done with PheB 1-PEG (600)-insulin in dogs showed an even more protracted action profile. These two kinds of insulin conjugates represent new potential candidates for soluble basal insulin preparations.

Insulin Analogs with Improved Pharmacokinetic Profiles (Brange & Volund 1999)

The aim of insulin replacement therapy is to normalize blood glucose in order to reduce the complications of diabetes. The pharmacokinetics of traditional insulin preparations, however, do not match the profiles of physiological insulin secretion. Peak absorption of regular, short-acting human insulin occurs from 2 to 4 hours after injection, usually persist for several hours, and does not provide the early and quick rise in plasma insulin concentration required to prevent unphysiological postprandial hyperglycemia after a meal. The protracted acting formulations, intended to supply basal insulin levels to control blood glucose between meals and during the night, are not capable of delivering insulin at a constant and reproducible low-level rate that characterize normal insulin secretion. These shortcomings of conventional preparations make it virtually impossible to achieve normoglycemia, thus aggravating the development of chronic complications.

In addition, insulin has a propensity to aggregate into dimers and hexamers at high insulin concentrations. This is not necessary for the biological activity of the hormone, as insulin binds to its receptor as a monomer. A summary of research efforts outlining the principles and strategies for creating insulin analogs is shown in Table 1. As seen from the table, the effort was directed in two areas: (a) creation of rapid-acting analogs, and (b) protracted-acting analogs. The success of creating rapid-acting analogs is evident from availability of two commercial preparations: Novorapid® and Humalog®. These monomeric insulin analogs are allowing patients improved postprandial glycemic control, greater convenience by permitting the patient to inject insulin closer to meals, and more flexibility in meal composition. On the other hand, the progress and delivery of insulin analogs for basal insulin delivery has been slow. The most recent design of protracted-acting insulin that holds promise for the future is fatty acid acylated insulin with albumin-binding properties.

Oral Delivery of Insulin

Oral delivery of insulin is most desirable from the viewpoint of patient compliance and comfort. However, it is also the most challenging route of administration. The details of oral delivery including the barriers for oral delivery of proteins in general, and insulin in particular, have been summarized below.

Enzymatic Barrier

Enzymatic barrier for oral delivery of proteins has been reviewed (Lee 1991; Langguth et al. 1997). Digestion of peptides is a natural phenomenon that is mediated by proteolytic enzymes present throughout the gastrointestinal tract (GIT). These proteolytic enzymes occur in the stomach, intestinal lumen and brush border that includes all the enzyme activity to the serosal side of the epithelium. Dietary proteins are initially digested to polypeptides by pepsin in the acidic pH of the stomach, and digestion is continued in the small intestine by proteolytic enzymes from pancreatic secretions containing trypsin, α-chymotrypsin, elastase and carboxypeptidase A.

TABLE 1

| | | Method used to | |
|---|---|---|---|
| Inhibitor | Activity against | establish efficacy | Reference |
| Pancreatic inhibitor | Not tested | In vitro enzymatic | (Laskowski et al 1958) |
| | | degradation and in vivo | |
| | | absorption in rats | |
| Soybean trypsin inhibitor | Not tested | In vivo studies in rats | (Laskowski et al 1985) |
| KF-448 | Chymotrypsin | In vitro enzymatic | (Fujii et al 1985) |
| | | Degradation and in vivo | |
| | | absorption in rats | |
| Soybean trypsin inhibitor | Not tested | In vivo absorption in | (Kidron et al 1982) |
| | | rats | |
| Aprotinin | Not tested | In vivo absorption in | (Bendayan et al 1990) |
| | | rats | |
| (Aprotinin, soybean trypsin | Small intestine and | In vitro stability and in | (Yamamoto et al 1994) |
| inhibitor | large intestine | vivo absorption | |
| | homogenates | | |
| Soybean trypsin inhibitor | Not tested | Oral absorption in dogs | (Ziv et al 1994) |
| 1,10,phenanthroline | Insulin degrading | Homogenates of | (bai & Chang 1996) |
| | enzyme | subcellular fractions | |
| p-chloromericuri benzoate | IDE | Homogenates of | (Bai & Chang 1996) |
| | | subcellular fractions | |
| Chymostatin | | | (Bai & Chang 1996) |
| Leupeptin | | | (Bai & Chang 1996) |
| Diisopropylphospho | Sesrine proteases | | (Bai 1995) |
| fluoridate | | | |
| Bacitracin | IDE | Homogenates of small | (Bai 1995) |
| | | intestinal epithelium | |
| Camostat | Serine proteases | Colon homogenates | (Tozaki et al 1997) |

Peptides with two to three amino acids are absorbed by the enterocytes. Peptides containing more than three amino acid residues are hydolyzed further by the aminopeptidases located at the brush border membrane of the enterocyte. Inside the enterocytes, the peptides are further hydrolyzed into free amino acids by cytosolic endopeptidases. Brush border and cytosolic enzymes include aminopeptidases A and N, diaminopeptidase IV, angiotension-converting enzyme and Gly-leu peptidase. The brush border membrane proteases prefer to cleave dipeptides, tripeptides and tetrapeptides. The cytosolic enzymes prefer dipeptides and oligopeptides.

The proteolytic enzymes cleave peptide bonds at specific locations within the polypeptide backbone. α-chymotrypsin cleaves peptide bonds near hydrophobic amino acids such as leucine, methionine, phenylalanine, tryptophan and tyrosine. Trypsin cleaves peptide bonds near basic amino acids such as arginine and lysine. Elastase cleaves peptide bonds near alanine, glycine, isoleucine, leucine, serine and valine. These proteases have an optimal activity at pH 8. Substrates for carboxypeptidase A include a free terminal carboxy group and a C-terminal amino acid bearing a branched aliphatic or an aromatic group.

The rate of proteolytic reactions is dependent on the pH of the environment and the substrate concentration. Environment pH has implications on enzyme activity. The activity of pepsin is highest in the acidic environment of the stomach and that of trypsin and α-chymotrypsin is optimal at alkaline pH. The concentration of substrate also plays an important role in determining its susceptibility to enzymes in accordance with the theory of enzyme substrate reactions. The concentration of the substrate in vivo may vary depending on factors such as receptors and endogenous protease inhibitors.

Enzymatic Degradation of Insulin

Insulin has been screened for its enzymatic degradation. Using everted gut sacs, it has been found that insulin did not undergo significant degradation when exposed to mucosal or serosal tissues (Schilling & Mitra 1990), but degraded significantly in whole tissue homogenates. This indicates that insulin is degraded by pancreatic and cytosolic enzymes. The pancreatic enzymes that degrade insulin extensively are trypsin and α-chymotrypsin (Ginsburg & Schachman 1960; Young & Carpenter 1961).

The kinetics of degradation and site of cleavage in the insulin molecule has been reported in the presence of trypsin and chymotrypsin (Schilling & Mitra 1991). It was found that the rate of degradation of insulin in the presence of chymotrypsin is about 10 times that in the presence of trypsin. The cleavage sites identified in the case of trypsin was B29-Lys and B22-Arg. The degradation of insulin in the presence of chymotrypsin generates four metabolites in a sequential and parallel manner. Chymotrypsin first cleaves at B26-Tyr to form Metabolite A and Metabolite B is formed by action on A19-Tyr. Metabolites C and D are formed by action on A14-Tyr and B16-Tyr. The cytosolic enzyme that degrades insulin is Insulin Degrading Enzyme (IDE).

The stability of insulin depends on its associated state in solution. Consequently, agents that deaggregate insulin in solution will increase the rate of degradation. Agents such as chelators (Liu et al. 1991) (EDTA), bile salts (Li et al. 1992) (sodium glycocholate) and surfactants (Shao et al. 1993) (sodium dodecyl sulfate, hexacedyl trimethylammonium bromide, Tween 80 and polyoxyethylene 9 lauryl ether) increase the rate of degradation of insulin by dissociating it into monomers.

Intestinal Epithelial Barrier

Epithelial Barrier for Proteins

The epithelial barrier for protein and peptide drug delivery has been reviewed extensively (Baker et al. 1991). In an average adult human, the small intestine is approximately 280 cm long and 4 cm in diameter. Three anatomical differences increase the effective area of the small intestine in a dramatic way. The folds of kerckring are spiral or circular concentric and folds up to 1 cm in height and 5 cm in length. These folds contain microscopic mucosal villi superimposed between them. The villi increase the absorptive surface area between 7 to 14 fold. The villi are present throughout the small intestine and exhibit size and shape variations from the duodenum to ileum. Each villi is covered by a layer of columnar absorptive cells and a few goblet cells. Absorptive cells have a series of projections on the apical side called microvilli. The microvilli are collectively known as brush border and they increase the absorptive surface by 14 to 40 fold.

The epithelial cells of the small intestine are absorptive cells, undifferentiated crypt cells, M cells and goblet cells. The columnar epithelial cells of the small intestine are highly polarized and their main function is absorption. The apical side consists of numerous closely packed microvilli. Immediately below is a narrower area called the terminal web which is clear in appearance and lacks in cytoplasmic organelles. The epithelial cells are joined at intercellular attachment zones or functional complexes which are 0.5 μM to 2 μM wide. The elements of this complex are known as zonula occludens or tight junctions, zonula adherens or intermediate junctions, and macula adherens or spot desmosomes. The tight junctions are present between the lateral membrane of adjacent cells at the apical end completely occupying the intercellular space. The structure of tight junctions varies with the region, cell type and position along the crypt-villus axis. The intermediate junctions are located below the tight junctions and appear on the site of insertion for the filaments of terminal web. Desmosomes are located in the functional complex and along the lateral membrane. They stabilize the cytoskeletal network of the epithelial cells.

The major functions of the crypt cells are proliferation and secretion. After they are formed in the crypt, many undifferentiated daughter cells undergo differentiation into villus cells as they migrate from the crypt to the villus. The undifferentiated crypt cells can also differentiate into goblet cells, Paneth cells and endocrine cells which are primarily secretory in function.

M (microfold cells) are specialized epithelial cells contained in the Peyer's patches. Peyer's patch is a group of subepithelial, lymphoid follicles distributed randomly throughout the small intestine. The difference with respect to columnar epithelial cells is the presence of sparse microvilli and less mucus. M cells perform sampling and transport of undegraded luminal particles and macromolecules into lymphoid follicles for immunologic surveillance and initiation of appropriate immunologic response. They have been investigated for the uptake of polymeric microparticles containing macromolecules.

Methodology to Study Intestinal Transport and Metabolism

Intestinal transport and metabolism can be studied using in vitro, in situ and in vivo techniques. An extensive summary of details of various methods may be found elsewhere (Baker et al. 1991; Ungell 2000).

In Vitro Methods

1. Everted Intestinal Rings (Slices)

The intestine of the animal is cut into rings or slices of approximately 30-50 mg (2-5 mm width) and put into an incubation media that is being agitated and oxygenated. Such studies are usually done for a short period of time. Samples of incubation media and rings are analyzed for drug content. This method has been used for the kinetic analysis of carrier-mediated transport of amino acids and peptides.

2. Everted Intestinal Sac

In this technique, a small segment of the intestine (2-3 cm) is tied at the ends after evertion on a glass rod. The mucosa faces the outer buffer solution and the serosa becomes the inside of the sac. An oxygenated buffer solution is injected into a sac and then it is immersed into a beaker container drug of interest. Samples of fluid are taken from the buffer solution in the beaker. This method has been used to determine permeability of drugs.

3. Brush Border Membrane Vesicles (BBMV)

This method is based on the homogenization of an inverted frozen intestine to give a purified fraction of the apical cell membranes from the chosen part of the gastrointestinal tract. This is frequently used for isolated studies of the brush-border membrane transport characteristics.

4. Cell Culture Techniques

The use of Caco-2 cell monolayers and other cell cultures (HT29, IEC-18) from the human carcinoma has been extremely popular. They consist of a monolayer of polarized cells grown on a filter support. After the cells are fully differentiated they express the transport characteristics of villus cells. The transport characteristics of drugs are studied in the inserts or by mounting them in side-by-side diffusion cells. Excellent correlations have been obtained between permeability coefficients generated on Caco-2 and absorption in humans.

In Situ Methods

1. Isolated Perfusion or In-Situ Perfusion

In these methods, a 10-30 cm segment of the intestine is cannulated on both ends and perfused with a buffer solution with a low flow rate (0.2 mL/min). The blood side is also cannulated through the mesentric vein and artery. Either the in and out concentrations of the perfusion solutions may be monitored or the appearance and disappearance on both sides of the membrane by monitoring the concentration of drug in blood. This technique has been applied to determine the absorption of drugs.

In Vivo Methods

In vivo methods include bioavailability in different animal models and man, intestinal perfusions in man and triple lumen perfusions. These studies are done early in the clinical phase in order to obtain complete understanding of the absorption of a certain drug, for information on the pharmaceutical dosage form program, and correlation of data obtained from simple animal models.

Mechanism for Protein Transport Across the Intestinal Tract

Passive diffusion and carrier-mediated transport of proteins across the intestinal tract is not reported. Cellular internalization of proteins occurs by an endocytotic process. There are two different pathways of endocytosis: (a) fluid phase type (non specific endocytosis, pinocytosis) and (b) adsorptive or receptor-mediated type (specific endocytosis).

Fluid phase endocytosis (FPE) is a process by which macromolecules dissolved in the extracellular fluid are incorporated by bulk transport into the fluid phase of endocytotic vesicles. Adsorptive endocytosis involves binding of macromolecule to the plasma membrane. Receptor mediated endocytosis (RME) is a subset of adsorptive endocytosis, whereby endocytosis occurs after binding of the macromolecule to the specific receptor present on the membrane. The three endocytotic processes discussed expose the macromolecule to the enzymes of lysosome before being transported. Transcytosis is a type of RME where the macromolecule transported is not exposed to the lysosomes.

Among the endocytotic processes, receptor-mediated endocytosis is the most important. This allows the absorptive cells of the small intestine to select and transport specific molecules while excluding undesirable or potentially harmful ones. Thus, the intestinal mucosa interacts with endogenous and foreign macromolecules (depending on their molecular structure) through the receptors present on the surface and facilitates internalization. Some examples of proteins transported by the endocytotic pathway are insulin (Kidron et al. 1982), trypsin and chymotrypsin (Ambrus et al. 1967).

Paracellular transport involves the control of movement of water and ions and prevention of passage of macromolecules through functional complexes. The absorption of water by the intestinal epithelium occurs by this route. It was hypothesized that this water could carry dissolved drugs and macromolecules that would not otherwise travel the apical membrane. It was demonstrated that glucose-induced increase in absorption of water carried [3H] methoxyinulin and [¹⁴C] polyethylene glycol 4000 across rat intestinal mucosa. In the absence of such increase in water transport, the transmucosal movement of these compounds was negligible (Munck & Rasmussen 1977).

Intestinal Transport of Insulin

Morpho-cytochemical and biochemical evidence for insulin absorption was demonstrated in the rat GIT (Bendayan et al. 1994) (Bendayan et al. 1990). This was achieved by direct instillation of a solution of insulin into various parts of the GIT, followed by visualization with gold markers and immonoassay of insulin in blood. There is no evidence for the transport of insulin by the paracellular route. It was found that insulin gets adsorbed to the apical plasma membrane and gets internalized by endocytosis. It then reaches the basolateral plasma membrane via the endosomal pathway of small vesicles and gets secreted into the interstitial space. It is not clear if the internalization is due to the presence of insulin receptors on the surface of the epithelial cells. The presence of insulin receptors has been demonstrated in enterocytes on both apical and basolateral side (Bergeron et al. 1980) (Pillion et al. 1985) (Gingerich et al. 1987).

Permeability studies of insulin across isolated segments of the GIT have been done with an aim to evaluate the apparent permeability coefficient of insulin. The in vitro permeability studies also serve as screening tools to test the efficacy of absorption modifiers. In recent years the use of epithelial cells like Caco-2 and HT-29 has become very popular due to highly reproducible culture yields and high throughput screening.

Insulin permeability across various segments of the gastrointestinal tract has been studied using isolated segments of the various regions. A summary of the apparent permeability coefficients of insulin calculated in various regions of the GIT is given in Table 2. From the table it can be seen that there are regional differences in permeability across various regions of the GIT. This has been attributed to the histological difference between the various sites. Also, there is a significant difference in permeability coefficient between the same segment. This can be explained by differences in preparation of tissues, apparatus used, concentration of insulin employed in the donor compartment, and duration of the studies.

TABLE 2

| | | Pₐₚₚ × | | |
|---|---|---|---|---|
| Exp. Model | Species | 10⁷ cm/sec | Technique | Reference |
| Duodenum | Rat | 0.78 ± 0.54 | Everted gut sac | (Schilling & |
| | | 4.85 ± 0.99 | Ussing technique | Mitra 1990) |
| | | | | (Asada et al |
| | | | | 1995) |
| Jejunum | Rat | 1.385 ± 0.265 | Everted gut sac | (Schilling & |
| | | 12.27 ± 1.73 | Ussing technique | Mitra 1990) |
| | | | | (Asada et al |
| | | | | 1995) |
| Ileum | Rat | 1.54 ± 0.29 | Everted gut sac | (Schilling & |
| | | 10.50 ± 2.06 | Ussing technique | Mitra 1990) |
| | | 5.0 ± 2.0 | Ussing technique | (Asada et al |
| | | | | 1995) |
| Colon | Rat | 4.05 ± 1.09 | Ussing technique | (Asada et al |
| | | | | 1995) |

Manufacturing Stability Issues

Stability issues associated with proteins during fabrication of delivery systems have been reviewed (Johnson 2000). The activity of proteins is dependent on the three dimensional molecular structure. Fabrication methods for dosage forms may expose the proteins to harsh conditions that may alter their structure. This will have implications in efficacy and immunogenic response from the protein.

Protein stability and structure are affected by variables such as temperature, pH, solvent, solutes and crystallinity states of the protein. During the fabrication of proteins with polymers they may be subjected to physical and chemical degradation.

Physical degradation involves the modification of the native structure of protein to higher order structure (secondary, tertiary, or quarternary). It may be brought about by aggregation, adsorption, unfolding or precipitation. The primary structure of a protein determines the native secondary and tertiary conformation. In general, in globular proteins, hydrophobic residues are buried in the interior and hydrophilic residues are available for interaction with the acqueous solvent.

Denaturation refers to the loss of globular structure and leads to protein unfolding, the extent of which may or may not result in the loss of secondary structure. Once unfolded, the protein may adsorb to surfaces by exposing the hydrophobic residues or the protein may aggregate by interaction with other protein molecules. Denaturation is caused by changes in the environment of the protein such as temperature, pH changes, the introduction of interfaces by the addition of organic solvents, or the introduction of hydrophobic surfaces. Chemical degradation usually involves bond cleavage and leads to the formation of a new product. Chemical degradation is usually preceded by a physical process such as unfolding which exposes the hidden residues to chemical reactions. The processes involved in chemical degradation are:

Deamidation-Hydrolysis of side chain amide on glutamine and asparagine residues to yield a carboxylic acid;

Oxidation-Tryptophan, methionine, cysteine, histidine and tyrosine are susceptible to oxidation in solution state and solid state. The source of oxygen may be atmospheric, flourescent, or hydrogen peroxide;

Disulfide exchange-Cysteine residues are involved in disulfide bond formation. Incorrect linkage of disulfide bonds leads to a change in three dimensional activity of the protein;

Hydrolysis-Aspartic acid residues have been implicated in the cleavage of peptide bonds which in turn has led to a decrease in biological activity.

After fabrication, there is a possibility of an interaction between the delivery matrix and the protein.

Strategies for Oral Delivery of Insulin

Oral insulin delivery involves overcoming the barriers of enzymatic degradation, low epithelial permeability, and taking steps to conserve bioactivity during formulation processing. The use of enzyme inhibitors, absorption enhancers and polymeric systems have been tried to overcome these barriers. These approaches will be discussed individually in the following sections.

Enzyme Inhibitors

The use of protease inhibitors has been tried with the goal of slowing the rate of degradation of insulin. It was hypothesized that a slow rate of degradation will increase the availability of insulin available for absorption. Enzymatic degradation of insulin, as discussed above, is mediated by the serine proteases: trypsin, a-chymotrypsin, and thiol metalloproteinase IDE. Consequently, excipients that inhibit these enzymes have been used for studying the stability of insulin in the presence of these enzymes. Inhibitors successfully used to increase the stability of insulin and enhance the bioavailability are listed in Table 1.

From the table it can be seen that a wide array of inhibitors have been tested using in vitro stability studies in small intestine and large intestine homogenates, Caco-2 cell monolayers and in vivo experiments. Comparison of efficacy of inhibitors has not been systematically studied. A class of inhibitors that has not been extensively evaluated for oral delivery of proteins is ovomucoids.

Some efforts in the prior art have focused on the use of ovomucoids as protein degradation inhibitors, but use very high concentrations of the enzyme inhibitor, leading to inhibitor toxicity problems. These attempts in the prior art involve a dosage form which consists of a polymer to which the proteolytic inhibitor and the biologically active agent are covalently attached. Such a dosage form modifies the biologically active protein, resulting in efficacy and release problems. Moreover, the inhibitor is not controllably released, which may result in sporadic protection of the protein and increased risk of inhibitor-related toxicity. There is a need in the art for a dosage formulation which provides for the controlled release of both the protein and the inhibitor, wherein the protein and inhibitor are not covalently bound or attached to the polymer. Such a formulation would provide localized protection of the protein and enhanced bioavailability. Additionally, there is a need for the release to be targeted to specific areas of the GIT, with prolonged and gradual release of the inhibitor in order to avoid inhibitor-related toxicity.

Ovomucoids

Ovomucoids are glycosylated proteins derived from the egg white of avian species. Extensive reviews of their source, method of isolation and mechanism of inhibitory activity is documented (Laskowski et al. 1990; Laskowski et al. 1987; Laskowski, Jr. & Kato 1960; Laskowski et al. 1958). They exhibit inhibitory action against various enzymes such as trypsin, α-chymotrypsin, subtilisin and elastase that is species dependent. Also, some ovomucoids have inhibitory action against one enzyme (single headed) and some have action against three enzymes (triple headed). These differences were explained by sequencing the domains of ovomucoids. It was found that ovomucoids contain three homologous domains, in which the residue of the reactive site varies widely. The connecting peptide between second and third domain can be readily hydrolyzed, and the resultant domain II and the double domain I-II are independently active. The carbohydrate in ovomucoid is attached to the Asn residues in the Asn-X-Thr/Ser sequence.

The mechanism of inhibitory action of ovomucoids is a standard mechanism shared by inhibitors of serine proteases. The reactive site of the inhibitor molecule specifically interacts with the active site of the cognate enzyme. This leads to hydrolysis of the peptide bond by the cognate enzyme at neutral pH. The hydrolysis reaction is extremely slow, does not proceed to completion and the system behaves as if it were a simple equilibrium between the enzyme and the free inhibitor on one hand and the complex on the other. At neutral pH the equilibrium constant between modified inhibitor (reactive site peptide bond hydrolyzed) and virgin inhibitor (reactive site peptide bond intact) is near unity. Therefore, both the modified inhibitor and virgin inhibitors are thermodynamically equal strong inhibitors of the cognate enzyme. However, the rate of complex formation between modified inhibitor and cognate enzyme is much slower than from virgin inhibitor and enzyme.

Apart from inhibitory action towards proteases, ovomucoids have the ability to interact with lectins due to the presence of a carbohydrate moeity. These two properties make them interesting from a pharmaceutical point of view. Ovomucoids, immobilized on polymers, have been used for enantioselective separation on HPLC columns (Haginaka et al. 1995), and preparation of gels (Plate et al. 1993).

Permeation Enhancers

Permeation enhancers improve the absorption of proteins by increasing their paracellular and transcellular transport. Increase in paracellular transport is mediated by modulation of tight junctions of the cells and increase in transcellular transport is associated with increase in fluidity of the cell membrane. Permeation enhancers that fall in the first class include calcium chelators and that in the second class include surfactants. Calcium chelators act by inducing calcium depletion causing global changes in the cells, including disruption of actin filaments, disruption of adherent junctions and diminished cell adhesion (Citi 1992). Surfactants act by causing exfoliation of the intestinal epithelium compromising its barrier functions (Hochman & Artursson 1994). This raises questions about their toxicity and long term clinical use. The majority of studies in the literature on the use of these agents have demonstrated that their enhancement is dose and time dependent. The ideal permeation enhancer would have a significant enhancement in permeation that is completely reversible. Examples of permeation enhancers used in the oral delivery of insulin are shown in Table 3.

TABLE 3

| Permeation | MOA | Method used to | | |
|---|---|---|---|---|
| Enhancer | category | establish efficacy | Concentration | Reference |
| Sodium laurate | Surfactant | Oral administration of | 4 mg:16 mg in | (Touitou & |
| and cetyl alcohol | | capsule in normal rats | 100 mg arachis | Rubinstein |
| in arachis oil | | | oil | 1986) |
| Sodium cholate | Surfactant | Oral administration of | 123 mg | (Ziv et al |
| | | capsule in diabetic dogs | | 1994) |
| EDTA | Calcium | Diffusion studies across | 5 mM | (Bai & Chang |
| | chelator | isolated tissues in using | | 1996) |
| | | chamber | | |
| Sodium cholate | Surfactant | Oral administration of | 50 mg | (Hosny et al |
| | | capsule in diabetic | | 1995) |
| | | rabbits | | |
| Sodium cholate | Surfactant | Oral administration of | 10 mg | (Trenktrog et |
| | | pellets in diabetic rats | | al 1996) |
| Sodium | Surfactant | Oral administration of | | (McPhililps et |
| deoxycholate | | capsules in normal rats | | al 1997) |

Formulation Approaches

Research in formulation development of proteins is focused in two directions: extended release and protection from the enzymes in the GIT. This has been possible due to the availability of functional polymers; release at specific pH, and inhibition of proteolysis. Formulation approaches with polymers alone have not been popular. Typically, a polymeric dosage form would have an absorption modifier such as enzyme inhibitor and/or permeation enhancer. Absorption modifiers have been discussed above. The formulation development approaches can be divided into functional categories: 1. Formulations targeted to bypass the stomach with an aim to release the drug in the intestine; 2. Formulations targeted to bypass the stomach and small intestine with an aim to release the drug in the colon; and 3. Formulations intended to extend the residence time in the GIT by providing bioadhesion to the intestinal wall. These approaches use the properties of functional polymers. Formulations in the first category are designed based on the pH differences in the gastrointestinal tract. The pH of the gastrointestinal tract changes drastically from the stomach to the small to the large intestine with a median value of pH 1.2 in the stomach to a range of pH 6-7.5 in the small and pH 7-8 in the large intestine.

The objective of successful protein delivery is to avoid protein degradation in the stomach due to harsh pH conditions and the presence of proteolytic enzymes. Functional polymers that dissolve at specific pH values can be used to manipulate the release of the active drug to achieve targeted release. Some examples are discussed below.

1. Methacrylic Polymers—Eudragit L 100® and Eudragit S 100® are examples of polymers manufactured by Rohm Pharma, Germany. Eudragit L100 starts to dissolve at pH 6.0 and Eudragit S 100 starts to dissolve at pH 7.0. Both of these polymers can be mixed in an appropriate ratio to modulate release at specific pH (ex 1:1 mixture of EL100 and ES 100 will dissolve at pH 6.5). Eudragit offers the convenience of enteric coating and extended release. This is an advantage compared to polymers like cellulose acetate pthalate, hydroxy propyl methyl cellulose phthalate and poly vinyl alcohol pthalate that have the property of enteric coating only, making them unpopular in applications for protein formulations.

Eudragit has been used to prepare microcapsules that can be administered directly or provide enteric coating on dosage forms such as microcapsules, pellets, beads and capsules.

Microcapsule-based dosage forms of Eudragit—Microsphere-based dosage forms with Eudragit L 100 and S 100 have been reported (Morishita et al. 1992a; Morishita et al. 1992b; Morishita et al. 1993). The authors prepared Eudragit microspheres by dissolving insulin in 0.1N HCL and Eudragit in alcohol, pouring it in liquid paraffin and forming microspheres with the addition of gelatin solution. The microspheres were characterized for drug incorporation efficiency and dissolution studies. The efficacy of the microspheres was demonstrated with the use of trypsin inhibitor, bowman birk inhibitor and aprotinin in vitro stability experiments in the presence of trypsin and α-chymotrypsin and in vivo experiments in normal and diabetic rats. The authors could demonstrate protection from enzymatic degradation in vitro and improvement in bioavailability (control 0.9±0.3% vs inhibitor 3.4±0.6%). The limitations were that a large amount of coating of Eudragit was need on microspheres and the drug incorporation efficiency was low (78-80%).

Enteric Coated Dosage Forms of Eudragit—Eudragit can be applied as an organic coating solution or as an aqueous dispersion on capsules or beads containing the active substance. Organic coating solutions can be made by dissolving Eudragit in organic solvents such as acetone, isopropyl alcohol up to 10% w/v. Aqueous dispersions of Eudragit such as L30D-55 and Eudragit FS 30D are commercially available and can be used directly for enteric coating purposes. Coating has been applied to capsules containing plain drug in a capsule (Hosny et al. 1998; Hosny et al. 1997; Hosny et al. 1995) or has been applied to insulin polymeric dosage forms such as microcapsules, pellets and beads. The authors used microspheres made of fat and triglycerides (Geary & Schlameus 1993), pellets made of microcrystalline cellulose (Trenktrog et al. 1996) and beads loaded with insulin, (McPhillips et al. 1997) as base material to apply the enteric coating.

Formulations in the second category are designed with an intention to bypass the stomach and small intestine. The active drug is released in the colon. This approach is attractive because of the extremely low concentration of enzymes present in the colon. Functional polymers that dissolve due to the enzymes released by the microbial flora in the colon facilitate the drug release. Examples include azopolymer (Saffran et al. 1991; Saffran et al. 1986) and chitosan (Tozaki et al. 1997) coated dosage forms that were successfully used to improve bioavailability in animal models. Azopolymer is a relatively impervious terpolymer of styrene, hydroxyethylmethacrylate and N,N′-bis (β⁻styrylsulphonyl)-4,4′-diaminoazobenzene as a cross linking agent. When azopolymer coating gets exposed to the resident microflora in the upper part of the colon, the cross links between the polymers are broken, making it porous. This allows for the passage of water to extract the insulin. Chitosan is a high molecular weight cationic polysaccharide derived from naturally-occuring chitin in crab and shrimp shells by deacetylation. This compound is also degraded by microflora of the colon and has the additional property of mucoadhesion. These properties have been utilized to target chitosan-coated dosage forms containing insulin to the colon (Tozaki et al. 1997).

The third category of formulations rely on the adhesion of polymer to the mucosal surface of the intestine. Mucoadhesive polymers localize the dosage form at the site of absorption, thereby decreasing the distance between the released drug and the absorptive tissue which leads to reduced drug metabolism caused by luminally secreted proteases (Luessen et al. 1994). Polymers in this category include polyacrylic acid derivatives, carbomer (Carbopol 974P®,934P® and 971P®, BF Goodrich Company, OH) and polycarbophil (PCP, Novean®, BF Goodrich Company, OH). Polyacrylic acid derivatives have shown a broad range of enzyme inhibitory activity due to their tendency to bind divalent cations from the enzymes (Luessen et al. 1997). They have also shown improvement in intestinal permeability by modulation of tight junctions (Luessen et al. 1995). But it has been shown that the enzyme inhibitory activity of polyacrylic acid derivatives is reduced when the buffer capacity of the medium is increased (Bai et al. 1995).

Optimization Strategies for Oral Controlled Release Dosage Forms

Optimization strategies are extensively used in product development to study the effect of factors (formulation and process variables) on the chosen responses. It offers a scientific way of doing minimal procedures to establish relationships between factors and responses. These relationships serve as important predictive tools to examine the magnitude and extent of effect of factors on the response. It is also possible to study the interaction among factors, if any, on the response. The final product should meet the requirements from a bioavailability, practical mass production and product reproducibility standpoint. The following steps are involved in an optimization process:

1. Selection of Factors and responses;

2. Identification of the low and high levels of the factors;

3. Performing a statistically designed set of experiments;

4. Measuring the response of interest;

5. Optimization by placing constraints on the model: performingmathematical calculations and graphical observation using contour and 3D plots; and

6. Verification of the optimized formulation.

Experimental Designs

In selecting an appropriate design, experiments must be chosen such that (1) the entire area of interest is covered and (2) analysis of results allows for separation of variables. Hence a proper design improves the process yield (efficiency) and reduces data variability, development time, and cost. Experimental designs can be classified as first order and second order depending upon the relationship obtained between the dependent and independent variables.

First Order Designs: These designs are used to screen the effects of independent variables on the responses. The relationship obtained is given as:

Y=A₀+A₁X₁+A₂X₂+A₃X₃+ . . .

Where Y is the measured response, A₀ is the intercept, A₁, A₂ and A₃ are the coefficients of the factors X₁, X₂ and X₃ respectively. Some examples of first order designs that were successfully applied in pharmaceutical product development include Simplex Design (Shek et al. 1980), Placket Burman Screening design (Kamachi et al. 1995a) and Latin Square Design (Khan et al. 1995).

Second Order Designs: These designs provide the relationship between factors and responses as follows: Y=A₀+A₁X₁+A₁₁X₁²+A₂X₂+A₁₂X²²+A₁₂X₁X₂+ . . . where A₁₂ is the interaction coefficient of X₁ and X₂. At least three levels of the factors are required to construct the model and the number of experiments must be greater than or equal to the number of coefficients in the model. Some examples of second order designs successfully applied in pharmaceutical product development include Factorial designs (Schwartz et al. 1973), Box Behnken design (Karnachi & Khan 1996) and Face Centered Cubic design (Agarwal et al. 1999).

Box-Behnken design: This design is suitable for exploration of quadratic response surfaces and constructs a second order polynomial model. It helps in optimizing a process using a small number of experimental runs. The design consists of replicated center points and a set of points lying at the midpoints of each edge of the multidimensional cube that defines the region of interest. For example, for three factors at three levels, only 15 experiments are required as compared to 27 experiments for a full factorial design.

After selection of the appropriate design and performing the experiments, a mathematical relationship between the dependent and independent variables is generated. The calculations involved are simplified by transforming the values of the factors as follows:

X^{T}=(X-average of the levels)/(½*(difference of levels))

The transformed values are in the range of −1 to 1 for minimum and maximum values.

Optimization

Optimization of the formulation is done after establishing the polynomial equation. Optimization techniques include simple inspection, grid search method (Schwartz 2000), Lagrangian method (Former, Jr. et al. 1970), and computer optimization. The computer based optimization methods are most popular. Software-aided optimization has practical applications in formulation and process development as the optimization process is very involved both mathematically and graphically. A variety of programs such as SAS®, RS 1®, ECHIP®, X-STAT®, and STATGRAPHICS® are available.

Validation of Analytical Methods

Validation of an analytical method is the process by which it is established, by laboratory studies, that the performance characteristics of the method meet the requirements for the intended analytical applications. Typical analytical parameters used in assay validation are accuracy, precision, limit of detection, limit of quantitation, linearity and range. The following definitions have been adapted from Validation of Analytical Parameters discussed in USP 23.

Analytical Performance Parameters

Accuracy—The accuracy of an analytical method is the closeness of test results obtained by that method to the true value. It may be determined by applying that method to samples or mixtures of excipients to which known amounts of analyte has been added both below and above the normal levels expected in the samples. It is represented by percentage of analyte recovered by the assay.

Precision—The precision of an analytical method is the degree of agreement among individual test results when the procedure is applied repeatedly to multiple samplings of a homogenous sample. It may be determined by assaying a sufficient number of aliquots of a homogenous sample to be able to calculate statistically valid estimates of standard deviation or relative standard deviation.

Limit of Detection—It is the lowest concentration of analyte in a sample that can be detected but not necessarily quantitated, under the stated experimental conditions.

Limit of Quantitation—It is the lowest concentration of analyte in a sample that can be determined with acceptable precision and accuracy under the stated experimental conditions.

Linearity and Range—The linearity of an analytical method is its ability to elicit results that are directly, or by well defined mathematical transformation, proportional to the concentration of the analyte in samples within a given range. The range of an analytical method is the interval between the upper and lower levels of analyte (including these levels) that have been demonstrated to be determined with precision, accuracy and linearity using the method as written.

Characterization of Dosage Forms of Insulin

The incorporation of a protein in a drug delivery system exposes it to harsh processing conditions. There is a possibility that formulation excipients may also interact with the protein, thus altering its conformation. This may lead to a loss in potency and biological activity. Thus, it is important to characterize the protein once it is incorporated into a drug delivery system to test for conservation of biological activity. The test method has to be suitable for characterization of the protein in its pure form and also in the presence of excipients.

Proteins have to be characterized for change in conformation, size, shape, surface properties, and bioactivity upon formulation processing. Changes in conformation, size and shape can be observed by the use of spectrophotometric techniques, x-ray diffraction, differential scanning calorimetry, light scattering, electrophoresis, Ultracentrifugation and Gel Filtration. Changes in surface properties can be detected by the use of electrophoretic and chromatographic techniques. Changes in the bioactivity of the proteins can be observed by bioavailability studies. Selection of a particular technique is based upon sensitivity of the technique, the system under study, and availability of equipment. The interference by formulation excipients may also play a major role in selection of the characterization technique. Theory about selected techniques used for the characterization of proteins have been reviewed (Pearlman & Nguyen 1991; Hoffmann 2000). Characterization techniques for insulin in particular have been discussed below.

Reverse Phase Chromatography

The applications of RP-HPLC arise due to the nature of the interaction between the stationary phase and the surface of the protein. Separation by RP-HPLC involves interaction of the surface hydrophobic areas of proteins with alkyl-bonded stationery phase. Elution of adsorbed proteins is produced by an increasing gradient of organic modifier such as isopropanol or acetonitrile. An ion-pairing agent such as triflouroacetic acid (TFA) is added to minimize interactions between the protein and unreacted silanol groups on the stationery phase. Reverse Phase HPLC is routinely employed for analysis of insulin. It has also been used to separate insulin from desamido insulin, higher order aggregates and other derivatives (Smith et al. 1985). The USP RP-HPLC method is an acceptable alternative to the rabbit bioassay for insulin, except in the case of highly purified insulins.

Size Exclusion Chromatography

Size exclusion chromatography detects changes in size of the protein under formulation conditions. It was initially known as gel filtration where the applications were mostly on preparative scale. The advent of new packing materials has permitted the development of high-performance chromatography, extending its analytical utility. The analytical uses of gel filtration include protein molecular weight determination, characterization of higher-order aggregates in protein samples, and determination of equilibrium constants for self-association.

Separation of macromolecules in gel filtration occurs because different sized molecules diffuse into the column matrix pores to different extents during their passage along the column. Since smaller molecules diffuse into the pores more readily, they elute more slowly than do larger species. It is common to refer to separation of proteins to be based on the “size” of analytes, when in fact the separation also depends on shape of the proteins. This is because the shape also determines entry of the protein into the gel matrix.

Size exclusion chromatography with RP-HPLC was used to determine the formation of covalent insulin dimers with trace amounts of high molecular weight transformation products after microencapsulating insulin in a mixture of poly (DL-lactide-co-glycolide) and poly (L-lactide) (Shao & Bailey 2000).

Differential Scanning Calorimetry

Differential Scanning Calorimetry is useful to detect changes in the secondary and tertiary structure of proteins when incorporated in polymer matrices. As a protein is heated, the transition from native to folded state is accompanied by appearance of an endothermic peak on a DSC. The transition temperature, Tₘ, is analogous to the melting of a crystal and is affected by environmental conditions. A shift in Tm indicates change in the denaturation temperature. This is dependent on the conformation of the protein. DSC has been used to determine the denaturation endotherms of amorphous and crystalline insulin (Pikal & Rigsbee 1997).

Fourier Transform Infrared Spectroscopy (FT-IR)

FT-IR also provides an estimate of secondary structure composition (Susi & Byler 1986). This method uses special deconvolution methods to separate and integrate overlapping amide I infrared absorption bands associated with α-helix, β-pleated sheet, and random structures. In this method, the spectrum is related to the subtle effects of regular secondary structure on the energetics (vibrational frequency) of amide groups in the peptide linkage. FT-IR also has the advantage of being able to evaluate the structural aspects of the protein in solid state.

X-Ray Diffraction

The diffraction of x-rays by crystalline substances is of great analytical interest, since no two compounds would be expected to form crystals in which the three-dimensional spacing of planes is totally identical in all directions. A powdered sample will exhibit all possible lattice planes, and the diffraction of the sample will provide information on all possible atomic spacings of the crystal lattice. The pattern consists of a series of peaks at different angles. These angles and their intensities are correlated with the d-spacings (distance between two planes in a crystal) to provide a full crystallographic characterization of the powdered sample. X-rays have wavelengths in the range 10⁻⁸-10⁻⁶ cm which is sufficient to allow determination of interplanar distances between the molecules.

Powder x-ray diffraction chromatograms of proteins are not regularly done due to lack of crystallinity, low intensity peaks and the requirement of a large sample. X-ray diffractograms of insulin have been obtained with mixtures of lactose and mannitol to compare the effect of spray drying on the crystalline changes in insulin (Forbes et al. 1998).

From the above discussion, it can be inferred that a variety of characterization methods are available for characterization of proteins post processing. For insulin, RP-HPLC and SEC-HPLC would indicate changes in primary structure and DSC, FT-IR and x-ray diffraction would indicate changes in secondary and tertiary structure. Changes in primary structure are irreversible whereas denaturation may be reversible or irreversible.

Bioavailability Studies

The final proof of the efficacy of the formulation is testing for its bioavailability. Bioavailability studies are done on dosage forms to evaluate the rate and extent of absorption. The rate of absorption is more important for drugs given as a single dose. It may be obtained by either measuring the rate constant for absorption or by comparing the peak concentration and time to reach peak concentration. The rate at which a protein reaches the plasma compartment depends on the route of administration. The extent of absorption (F) is defined as the fraction of unchanged drug reaching the systemic circulation from a given route of adminstration. It is calculated by dividing the area under the concentration-time curve (AUC) obtained after administering the drug by a particular route by the AUC of a separate, equally-sized intravenous dose. Bioavailability (F) values less than unity can be attributed to one of the following reasons: (1) incomplete absorption, (2) metabolism at the site of adminstration, (3) metabolism in the liver prior to entry in the systemic circulation, and (4) incomplete reabsorption after enterohepatic cycling on oral administration.

Bioavailability studies on experimental formulations are usually done in animal species such as Rhesus monkeys, New Zealand rabbits, Sprague-Dawley rats, mongrel dogs among others. Extrapolation from one animal species to another needs to be made with caution as the different animal species may differ in their metabolic clearance rates and proteolytic activities. Also, the therapeutic protein or peptide should have the desired pharmacological effect in the animal species chosen. For example, human interferon-γ is not active in rats.

Bioavailability of experimental oral formulations of insulin has been evaluated in rats (McPhillips et al. 1997), rabbits (Hosny et al. 1998) and dogs (Ziv et al. 1994). Analysis of insulin in plasma was done by a radioimmunoassay method and the pharmacodynamic effect was evaluated by monitoring reduction in glucose.

### SUMMARY OF THE INVENTION

The present invention is directed to an oral dual controlled release formulation of a protein and inhibitor and to methods of preparing these compounds. Oral delivery of proteins may be enhanced by the use of absorption modifiers such as enzyme inhibitors and permeation enhancers. In the present study, ovomucoids were investigated as absorption modifiers in the oral delivery of model proteins, insulin and calcitonin. Ovomucoids are enzyme inhibitors isolated from egg white of avian species. They have protease inhibitory activity and also bind to lectins on the cell surfaces through their carbohydrate moeity. A preferred embodiment of the present invention is directed to a novel dual controlled release formulation of insulin and ovomucoid.

Enzymatic degradation studies reveal that insulin is degraded extensively in the presence of trypsin and α-chymotrypsin. Duck ovomucoid (DkOVM) stabilized insulin against degradation in the presence of trypsin and α-chymotrypsin for an hour. In contrast, chicken ovomucoid (CkOVM) was only effective against trypsin mediated degradation of insulin. Permeability studies of insulin from rat intestinal segments reveal that insulin is absorbed more from the jejunum and ileum than from the duodenum. In the presence of CkOVM and DkOVM, the permeability of insulin decreased, which may be explained in part by the action of insulin by adipocytes.

The coprecipitation technique for preparation of microcapsules of insulin was evaluated and dissolution stability experiments in the presence of trypsin and α-chymotrypsin using chicken and duck ovomucoids were performed.

After microencapsulation, further objects of the present invention include characterization of insulin by using DSC, FT-IR, x-ray diffraction and size exclusion chromatography:

To determine the non-linear relationship of certain important variables and drug release using Box Behnken design;

To elucidate drug release kinetics and release mechanism;

To optimize the drug release within the given set of constraints to get maximum response and verify the optimized formulations; and

To monitor the release of duck ovomucoid from representative formulations studied for insulin.

Thus, the present invention evaluated the role of chicken and duck ovomucoids as representative enzyme inhibitors for the oral delivery of insulin. Duck ovomucoid improved the stability of insulin in the presence of trypsin and α-chymotrypsin. Chicken ovomucoid was effective against trypsin-mediated degradation but not against α-chymotrypsin degradation. The inhibitory action of duck ovomucoid was reduced in the presence of deaggregating agents. The cumulative amount of insulin permeated at the end of three hours was comparable from the jejunum and ileum and was more than permeation from the duodenum. The permeability of insulin from the rat jejunum decreased in the presence of chicken and duck ovomucoid. The permeability of insulin improved in the presence of α-chymotrypsin and duck ovomucoid. The ovomucoids increased the permeability of a lipophilic (testosterone) marker and a hydrophilic (mannitol) marker in a concentration-dependent fashion, indicating that they have the ability to modulate the mucosal barrier of the small intestine.

Microencapsulation of insulin was possible using the coprecipitation technique with appropriate combination of factors and choice of polymer. The rate of addition of polymer to the precipitating medium and the ratio of precipitating medium with respect to the polymeric solution had an effect on the microencapsulation dissolution profile. Dissolution enzymatic stability of insulin improved in the presence of chicken ovomucoid and duck ovomucoid. Optimization of a tablet dosage form containing insulin and DkOVM using a three-factor three-level Box Behnken design yielded a formulation with 94% release at the end of 6 hours.

Constrained optimization was successfully applied to tailor the release of insulin at each point over time. Mathematical relationships, generated in the form of polynomial equations, explained the quadratic and interaction effects of the formulation factors on the dissolution of insulin. The predicted and observed values of the dissolution profiles from the Box Behnken design were in close agreement. The dosage form delayed the release of DkOVM. The various formulations indicated a range of dissolution profiles of DkOVM. Thus, the present invention discloses an oral dosage form characterized by the dual controlled release of insulin and ovomucoid.

Further studies involving the protein calcitonin show a third type of ovomucoid, turkey ovomucoid (TkOVM) is effective as an enzyme inhibitor in the presence of trypsin and α-chymotrypsin. Studies with insulin and duck ovomucoid (DkOVM) provide support for the in vivo bioavailablity and efficacy of the oral dosage formulation of the protein with inhibitor and demonstrated significantly enhanced hypoglycemic effect.

### BRIEF DESCRIPTION OF THE DRAWING FIGURES

A more complete understanding of the objects and processes of the present invention may be had by reference to the following detailed description taken in conjunction with the accompanying drawings, wherein:

FIG. 1 illustrates the primary structure of human insulin;

FIG. 2 is a graphic illustration of the mechanism of action of insulin;

FIG. 3 is a representative chromatogram of insulin and metabolites;

FIG. 4 is a graph illustrating the chymotrypsin mediated degradation of insulin vs. time in the absence of DkOVM and in the presence of DkOVM at enzyme inhibitor ratios of 1:0.5, 1:1, and 1:2;

FIG. 5 is a graph illustrating the trypsin mediated degradation of insulin vs. time in the absence of inhibitor and at enzyme to inhibitor ratio 1:1 using inhibitors DkOVM and CkOVM;

FIG. 6 is a graph illustrating the chymotrypsin mediated degradation of insulin vs. time in the absence of inhibitor and at enzyme to inhibitor ratio 1:1 using inhibitors aprotinin and DkOVM;

FIG. 7 is a graph illustrating the chymotrypsin mediated degradation of insulin vs. time, by itself and in the presence of EDTA and NaGC;

FIG. 8 is a graph illustrating the peak areas of Metabolite I and Metabolite II in the absence of DkOVM and in the presence of DkOVM;

FIG. 9 is a representative chromatogram of m-cresol and insulin;

FIG. 10 is a bar chart illustrating the cumulative amount of insulin released from various segments of the intestine at the end of three hours;

FIG. 11 is a graph illustrating the cumulative amount of insulin permeated (m.IU) vs. time in the absence of DkOVM and at DkOVM concentrations of 0.5 μM, 1.0 μM, and 1.5 μM;

FIG. 12 is a graph illustrating the cumulative amount (μ.Ci) of [7-³H] testosterone permeated vs. time in the presence of DkOVM at concentrations of 0.5 μM, 1.0 μM, and 1.5 μM;

FIG. 13 is a graph illustrating the cumulative amount (μ.Ci) of D-[1-¹⁴C] mannitol permeated vs. time in the presence of DkOVM at concentrations of 0.5 μM, 1.0 μM, and 1.5 μM;

FIG. 14 is a graph illustrating the cumulative amount of insulin permeated (m.IU) vs. time in the presence of α-chymotrypsin in the absence and presence of DkOVM at 1:1 and 1:2 ratio of enzyme to inhibitor;

FIG. 15 is a graph illustrating the chymotrypsin-mediated degradation of insulin as a function of time in the absence of DkOVM and at enzyme-to-inhibitor ratios of 1:1 and 1:2;

FIG. 16 is a graph illustrating a representative dissolution profile of a batch of microcapsules;

FIG. 17 is a graph illustrating the effect of salts in the precipitating medium on the dissolution of insulin microcapsules;

FIG. 18 is a graph illustrating the degradation of insulin solution (50IU in the presence of trypsin and α-chymotrypsin;

FIG. 19 is a graph illustrating the dissolution stability of insulin released from microcapsules in the presence of trypsin and CkOVM;

FIG. 20 is a graph illustrating the dissolution stability of insulin released from microcapsules in the presence of α-chymotrypsin and DkOVM;

FIG. 21 is a DSC thermogram of insulin powder;

FIG. 22 is a DSC thermogram of Eudragit L100;

FIG. 23 is a DSC thermogram of a physical mixture of insulin and Eudragit L100;

FIG. 24 is a DSC thermogram of microcapsules of insulin;

FIG. 25 illustrates powder x-ray diffractograms of insulin, Eudragit L100, physical mixture of Eudragit L100 and insulin, and microcapsules of insulin;

FIG. 26 illustrates FT-IR spectra of insulin, polymer, physical mixture of 2% insulin and polymer, physical mixture of 50% insulin and polymer, and insulin microcapsules;

FIG. 27 illustrates SEC chromatograms of insulin, physical mixture of insulin and Eudragit L100, and insulin extracted from microcapsules in pH 6.8 buffer;

FIG. 28 illustrates a representative chromatogram of insulin and duck ovomucoid;

FIG. 29 illustrates the dissolution profiles of insulin from formulations 1-5 of the experimental design of the present invention;

FIG. 30 illustrates the dissolution profiles of insulin from formulations 6-10 of the experimental design of the present invention;

FIG. 31 illustrates the dissolution profiles of insulin from formulations 11-15 of the experimental design of the present invention;

FIG. 32 is a graph illustrating the fitting of the dissolution kinetic models to the experimental formulations of the present invention;

FIG. 33 is a graph illustrating the theoretical profile of dissolution of insulin after fitting to the dissolution kinetics model;

FIG. 34 illustrates a contour plot showing the effects of rate of addition and compression pressure on cumulative amount of drug released at the end of 6 hours;

FIG. 35 illustrates a response surface plot showing the effect of rate of addition and compression pressure on cumulative amount of drug released at the end of 6 hours;

FIG. 36 illustrates a contour plot showing the effect of compression pressure and volume of water with respect to polymeric solution on cumulative amount of drug released at the end of 6 hours;

FIG. 37 illustrates a response surface plot showing the effect of compression pressure and volume of water with respect to polymeric solution on cumulative amount of drug released at the end of 6 hours;

FIG. 38 illustrates a contour plot showing the effect of rate of addition and volume of water with respect to polymeric solution on cumulative amount of drug released at the end of 6 hours;

FIG. 39 illustrates a response surface plot showing the effect of volume of water with respect to polymeric solution and rate of addition and on cumulative amount of drug released at the end of 6 hours;

FIG. 40 is a graph illustrating the comparison of observed and predicted dissolution profiles of the optimized formulation of insulin;

FIG. 41 is a graph illustrating the dissolution profiles of DkOVM from formulations 1-5 of the experimental design of the present invention;

FIG. 42 is a graph illustrating the dissolution profiles of DkOVM from formulations 6-10 of the experimental design of the present invention;

FIG. 43 is a graph illustrating the dissolution profiles of DkOVM from formulations 11-15 of the experimental design of the present invention;

FIG. 44 is a graph illustrating the evaluation of protease inhibitors (1:1 trypsin: inhibitor) against trypsin mediated sCT degradation;

FIG. 45 is a graph illustrating the effects of protease inhibitors (1:1 trypsin: inhibitor) on trypsin mediated sCT metabolite formation;

FIG. 46 is a graph illustrating the evaluation of DkOVM against trypsin mediated sCT degradation;

FIG. 47 is a graph illustrating the effects of DkOVM at various ratios on trypsin mediated sCT metabolite formation;

FIG. 48 is a graph illustrating the evaluation of TkOVM against trypsin mediated sCT degradation;

FIG. 49 is a graph illustrating the effects of TkOVM at various ratios on trypsin mediated sCT metabolite formation;

FIG. 50 is a graph illustrating the evaluation of protease inhibitors (1:1 α-chymotrypsin:inhibitor) against α-chymotrypsin mediated sCT degradation;

FIG. 51 is a graph illustrating the effects of protease inhibitors (1:1 α-chymotrypsin:inhibitor) on α-chymotrypsin mediated sCT metabolite formation;

FIG. 52 is a graph illustrating the evaluation of DkOVM against α-chymotrypsin mediated sCT degradation;

FIG. 53 is a graph illustrating the effects of DkOVM at various ratios on α-chymotrypsin mediated sCT metabolite formation;

FIG. 54 is a graph illustrating the evaluation of TkOVM against α-chymotrypsin mediated sCT degradation;

FIG. 55 is a graph illustrating the effects of TkOVM at various ratios on α-chymotrypsin mediated sCT metabolite formation;

FIG. 56 is a graph illustrating the evaluation of tOV against trypsin and α-chymotrypsin mediated sCT degradation;

FIG. 57 is a chart illustrating the degradation of sCT in Caco-2 cell and goat intestinal homogenates;

FIG. 58 illustrates the gel electrophoresis of sCT metabolites by trypsin and chymotrypsin;

FIG. 59 is an HPLC chromatogram of sCT;

FIG. 60 is an HPLC chromatogram of trypsin mediated sCT metabolite;

FIG. 61 illustrates chymotrypsin mediated sCT metabolite;

FIG. 62 illustrates sCT metabolites formed by trypsin and chymotrypsin;

FIG. 63 is an MS chromatogram of trypsin mediated sCT degradation and metabolite formation;

FIG. 64 is an MS chromatogram of chymotrypsin mediated sCT degradation and metabolite formation;

FIG. 65 is an MS chromatogram of trypsin and chymotrypsin mediated sCT degradation and metabolite formation;

FIG. 66 is a graph illustrating the dissolution profile of insulin and DkOVM from the optimized formulation; and

FIG. 67 is a graph illustrating the hypoglycemic effect of the various formulations: subcutaneous injection, tablet without DkOVM, and tablet with DkOVM.

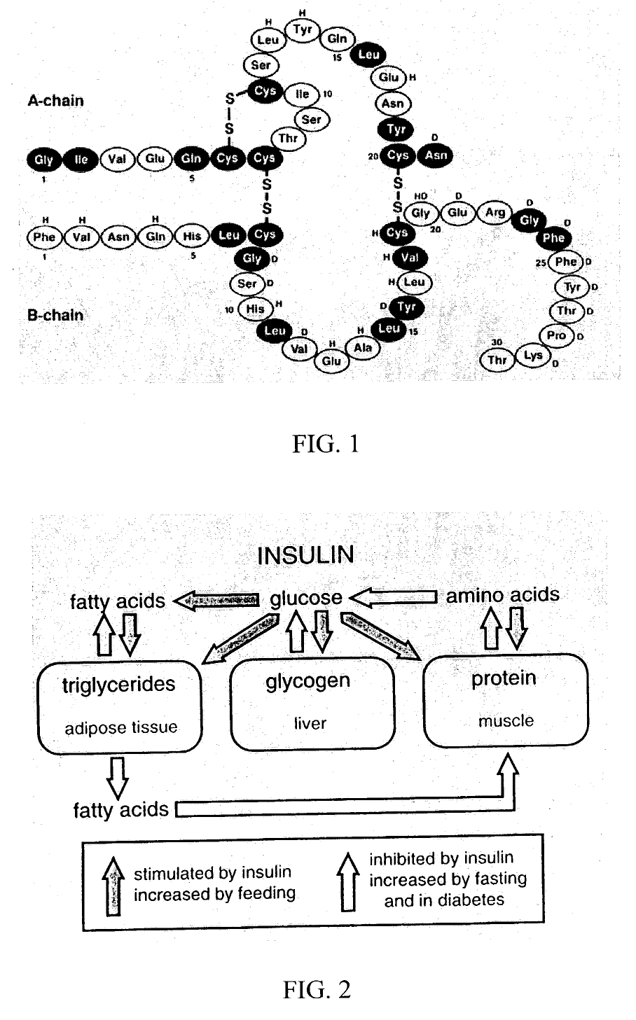

### DETAILED DESCRIPTION OF THE INVENTION

The features and details of the invention will now be more particularly described below and pointed out in the claims. It will be understood that the particular embodiments of the invention are shown by way of illustration and not as limitations of the invention. The principal features of this invention may be employed in various embodiments without departing from the scope of the invention.

An objective of a preferred embodiment of the present invention was to prepare and characterize a dual controlled release dosage form of insulin and duck ovomucoid for adminstration by the oral route. Insulin degradation studies with some common intestinal enzymes such as trypsin and α-chymotrypsin and enzyme inhibitor studies with certain naturally occurring inhibitors, ovomucoids, were performed. A further object of the present invention was to determine the site of maximum permeability of the model protein, insulin, in the small intestine and to evaluate the permeability of insulin in the presence of representative inhibitors, enzymes, and transport markers.

The present invention utilizes a coprecipitation technique to prepare microcapsules of insulin with high encapsulation efficiency. Dissolution stability studies of insulin microcapsules in the presence of enzymes reveal considerable improvement in the availability of insulin with ovomucoids even at the end of six hours. Characterization of insulin in the microcapsules using DSC, FT-IR, powder x-ray diffraction, and size exclusion chromatography revealed that the structure of insulin was conserved after subjecting it to formulation and process conditions. A three-factor, three-level optimization design was used to evaluate the effect of critical process variables including the rate of addition of polymeric solution, compression pressure, and volume of water with respect to polymeric solution. Mathematical relationships, contour plots, and response surface methods were employed with constrained optimization to predict levels of factors that provide optimum response. The predicted and observed values were in close agreement. The release of DkOVM was delayed from the formulation. The novel formulation incorporates controlled release characteristics of both protein and inhibitor to enhance the stability and availability of the protein with less potential for inhibitor concentration-related toxicity. The present invention utilized insulin as a model protein and chicken ovomucoid (CkOVM) and duck ovomucoid (DkOVM) as enzyme inhibitors. The dosage formulation developed utilizes pH sensitive Eudragit polymers to target the release of insulin in the small intestine.

### EXAMPLE 1

### Effect of Chicken and Duck Ovomucoid on Trypsin and A-Chymotrypsin Mediated Degradation of Insulin

Analysis of Insulin and Metabolites by Isocratic HPLC Method

Insulin powder was dissolved in 0.01N HCl to obtain a final concentration of 100 IU/mL. Diluted concentrations were made in 1% v/v.TFA/TRIS using this stock solution. Chromatography was performed under the conditions of isocratic HPLC method for analysis of insulin and degradation products as given in Table 4. The following parameters were evaluated for analytical validation. The minimum detectable quantity was determined by a S/N ratio of 4:1. The linearity range of standard curve for insulin was between 0.058 IU/mL-1.44 IU/mL (1 IU=34.84 μg).

TABLE 4

| | |
|---|---|
| Column | Jupiter C₁₈ (250 × 4.6 m.m) 5μ 300 Å |
| Mobile phase | Water 0.1% v/v TFA):Acetonitrile (0.1% v/v |
| | TFA) |
| Isocratic conditions | 30% B for 15 mins |
| Flow rate | 1 mL/min |
| Detection wavelength | 210 nm |
| AUFS | 0.001 |
| Injection volume | 100 μL |

The method precision was evaluated by injecting a standard concentration of insulin in six runs and computing the relative standard deviation (RSD) of the peak areas observed. Recovery studies were done by injecting known concentrations of insulin and comparing with the concentration estimated from the standard curve. The stock solution was stored in the refrigerator and diluted to 0.57 IU/mL each day and analyzed as per the method reported above on three different days. Interday variations in the peak area were observed.

Stability studies in the presence of chicken and duck ovomucoid were conducted. Insulin solutions at a concentration of 18 μM were incubated at 37° C. in 100 mM TRIS buffer and 1 mM calcium chloride (adjusted to pH 8.0). The degradation profiles were generated in the presence of 0.1 μM α-chymotrypsin and 0.5 μM trypsin over a period of 60 minutes that served as controls. These concentrations were selected based on a reported study (Schilling & Mitra 1991). Duck ovomucoid (DkOVM) and chicken ovomucoid (CKOVM) were evaluated at various ratios with respect to the enzymes to evaluate their protection of insulin degradation.

Insulin and enzyme solutions were incubated separately for a period of 15 minutes at 37° C. before starting the experiments. Samples were taken at 0, 5, 15, 30 and 60 minutes and immediately diluted with cold 1% TFA/TRIS to reduce the pH to 2.5. The samples were analyzed at 4° C. by a reverse-phase HPLC method using a Varian Chromatography Workstation. Plots of insulin remaining versus time were generated. I₆₀ (percent insulin remaining at 60 minutes) values were used to compare the efficacy of ovomucoids. Similar studies were performed with aprotinin.

Stability Studies in the Presence of EDTA and Sodium Glycocholate

The protocol used was the same as above with minor modifications. The concentrations of EDTA and NaGC were chosen so as to maximize deaggregation of insulin in solution (Liu et al. 1991; Li et al. 1992). The control solution had 0.05 mM EDTA and 30 mM NaGC in addition to insulin. The degradation profiles were generated in the presence of α-chymotrypsin and DkOVM. The enzyme to inhibitor ratio used was 1:1 with respect to α-chymotrypsin.

Results and Discussion of the Effect of Chicken and Duck Oyomucoid on the Trypsin and α-Chymotrypsin Mediated Degradation of Insulin

Analysis of Insulin and Metabolites by Isocratic HPLC Method

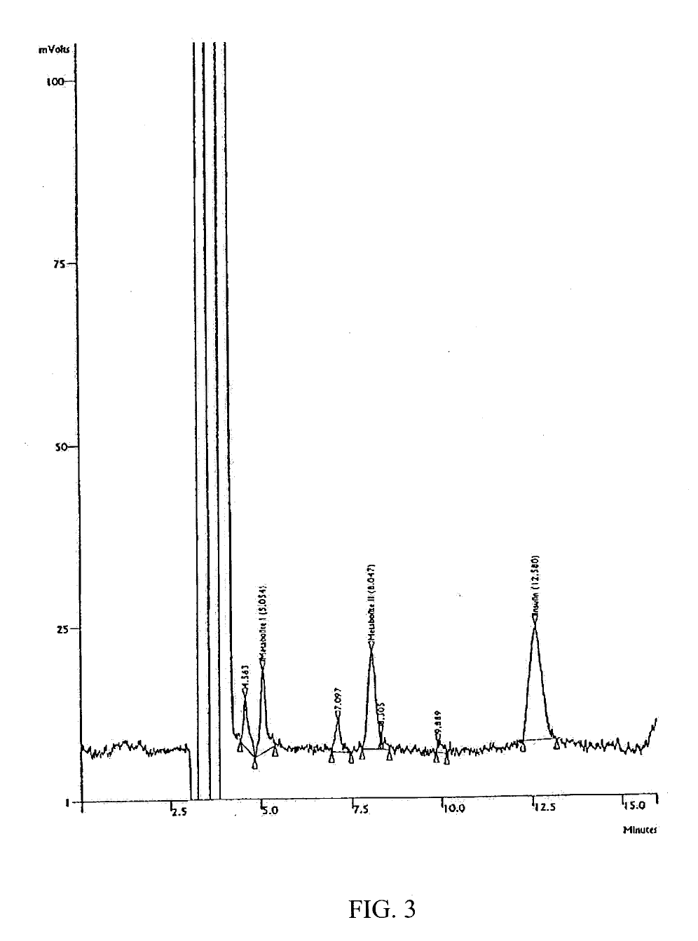

A typical chromatogram showing the elution of insulin, metabolite I and metabolite II is shown in FIG. 3. From the figure it can be seen that the elution times of insulin, metabolite I and metabolite II are 12.580 min., 5.054 min. and 8.047 min. The range of concentration for standard curve for the analysis of insulin was between 0.058 IU/mL-1.44 IU/mL. For this range, the slope value of a typical run was 17949.186 and intercept value was −2160.611. The correlation coefficient (r²) for this run was 0.999. The minimum detectable quantity at S/N ratio of 4:1 was 0.032 IU/mL. The results of the precision experiment at a standard concentration of 0.57 IU/mL are shown in Table 5. From the table it can be seen that the relative standard deviation is less than 0.32%. The results of the recovery studies are shown in Table 6. From the table it can be seen that the recovery of insulin is almost complete. The results of the inter-day variation study are shown in Table 7. From the table it can be seen that the interday variations were almost negligible for the first three days.

TABLE 5

| Run | Peak Area |
|---|---|
| 1 | 362586 |
| 2 | 365736 |
| 3 | 364022 |
| 4 | 364148 |
| 5 | 362641 |
| 6 | 363410 |
| Average | 363757.2 |
| Standard Deviation | 1172.8 |
| Relative Standard Deviation | 0.32% |

TABLE 6

| | Insulin added | Insulin recovered | |
|---|---|---|---|
| Conc # | (IU/mL) | (IU/mL) | Recovery % |
| 1 | 0.15 | 0.16 ± 0.01 | 106.0 |
| 2 | 0.29 | 0.29 ± 0.0005 | 100.0 |
| 3 | 0.57 | 0.57 ± 0.009 | 100.7 |
| 4 | 1.14 | 1.15 ± 0.02 | 100.8 |

TABLE 7

| | Insulin added | Insulin recovered | |
|---|---|---|---|
| Day # | (IU/mL) | (IU/mL) | Recovery % |
| 1 | 0.57 | 0.58 | 101 |
| 2 | 0.57 | 0.59 | 101.5 |
| 3 | 0.57 | 0.57 | 100 |

Trypsin and α-Chymotrypsin Degradation of Insulin in the Presence of Ovomucoids

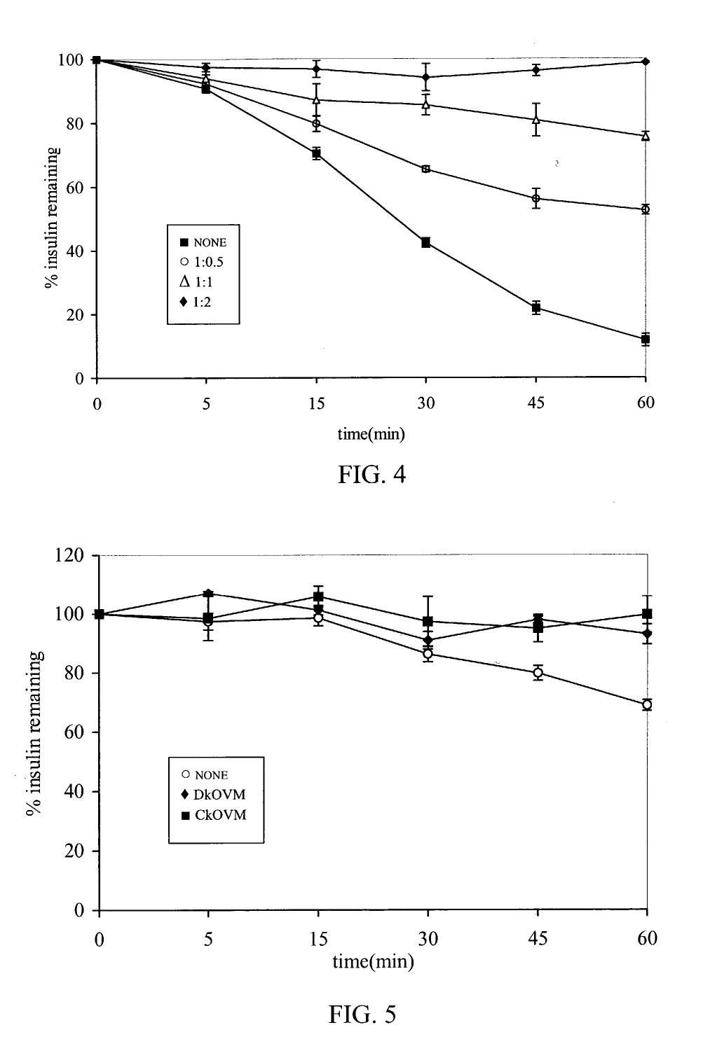

The degradation profile of insulin in the presence of α-chymotrypsin and various concentrations of DkOVM are shown in FIG. 4. The figure illustrates chymotrypsin mediated degradation of insulin vs. time in the absence of DkOVM and in the presence of DkOVM at enzyme inhibitor ratios 1:0.5, 1:1, and 1:2. The values represent the average of at least three independent experiments.

The control experiment shows that more than 90% of insulin degraded in 60 minutes in the presence of α-chymotrypsin. When the inhibitor (DkOVM) was added, the degradation decreased. The extent of degradation decreased as the enzyme to inhibitor ratio was increased. At an enzyme to inhibitor ratio of 1:2, %I₆₀ₘᵢₙ (percent of insulin remaining at the end of 60 minutes) was 98.77(±2.33) when compared to the control value (Table 8). The extent of degradation by α-chymotrypsin was not affected by the presence of CkOVM. Even at an enzyme to inhibitor ratio of 1:4, %I₆₀ₘᵢₙ was 9.47(±0.27) when compared to control (Table 9, illustrating the percentage of insulin remaining at the end of 60 minutes in the presence of CkOVM). In contrast, both DkOVM and CkOVM were effective 100% against trypsin mediated degradation of insulin at ratio 1:1 as indicated by the comparable value of %I₆₀ₘᵢₙ with control (see FIG. 5; Table 8; Table 9). FIG. 5 illustrates trypsin mediated degradation of insulin vs. time in the absence of inhibitor and at enzyme to inhibitor ratio 1:1 using inhibitors DkOVM and CkOVM. The values represent the average of at least three independent experiments.

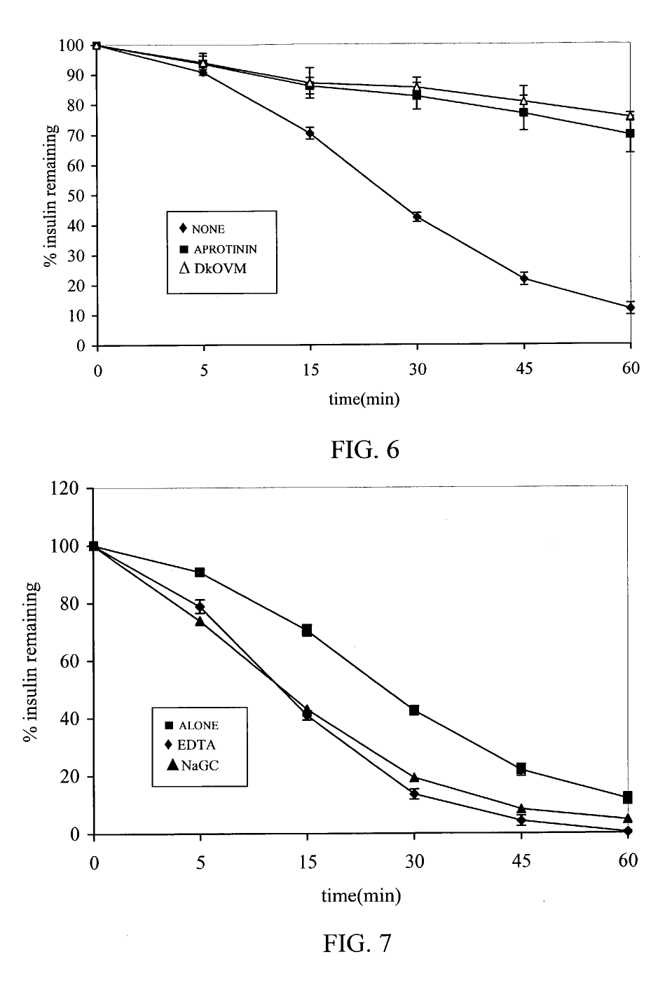

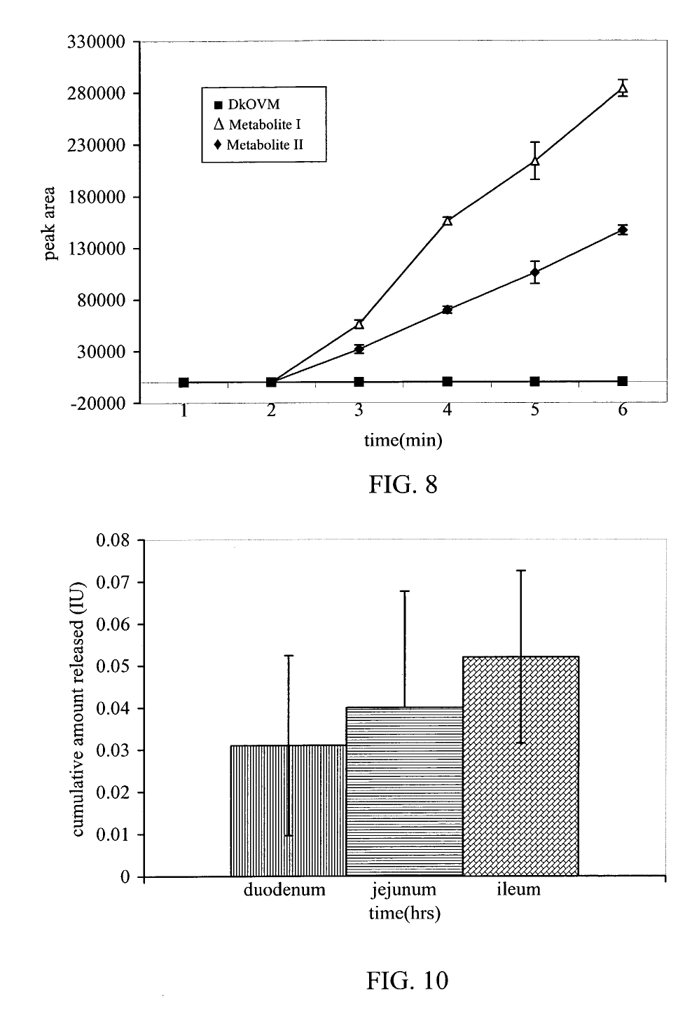

This effect was comparable to the effect of aprotinin at 1:1 ratio with respect to trypsin and α-chymotrypsin in the presence of DkOVM (FIG. 6, Table 8). FIG. 8 illustrates chymotrypsin mediated degradation of insulin vs. time in the absence of inhibitor and at enyzme to inhibitor ratio 1:1 using inhibitors aprotinin and DkOVM. Values represent the average of at least three independent experiments.

It is clear that the inhibitory action of ovomucoids is enzyme and species dependent. Ovomucoids belong to the pancreatic secretory trypsin family of inhibitors (Laskowski, Jr. & Kato 1960). Briefly, each inhibitor molecule has at least one peptide bond known as the reactive site that interacts with the corresponding enzyme. The reactive site reacts with the enzyme through van der Waals interaction, salt bridges and-hydrogen bonding. CkOVM has only one inhibitory site for trypsin, whereas DkOVM has two sites for trypsin and one each for chymotrypsin, subtilisin and elastase. Results show the inhibitory action of CkOVM and DkOVM using insulin as a substrate. Further inhibitory response curves were established as a function of concentration range (0.05 μM to 0.2 μM) for the inhibitors studied. Aprotinin is a non-specific protease inhibitor derived from bovine lung tissue and is associated with anti-fibrinolytic activity and preservation of platelet function (Robert et al. 1996). If it is administered orally, it undergoes gastric inactivation (Royston 1992).

Degradation in the Presence of Deaggregating Agents

The degradation of insulin mediated by α-chymotrypsin in the presence of deaggregating agents EDTA (0.05 mM) and NaGC (30 mM) is shown in FIG. 7. FIG. 7 illustrates chymotrypsin mediated degradation of insulin vs. time, by itself and in the presence of EDTA and NaGC. Values represent the average of at least three independent experiments.

Degradation of insulin increased significantly in the presence of both agents. The %I₆₀ₘᵢₙ values were at least 2-fold lower when compared to %I₆₀ min in the presence of α-chymotrypsin alone (Table 8). Subsequently, when insulin was incubated with DkOVM at enzyme to inhibitor ratio of 1:1 with DkOVM, the inhibitory activity decreased. The %I₆₀ₘᵢₙ values 57.89(±1.52) and 56.58(±4.84) obtained in the presence of EDTA and NaGC reflect a 17% and 19% decrease in inhibitory activity when compared to the %I₆₀ₘᵢₙ values in the presence of DkOVM at enzyme inhibitor ratio 1:1 (Table 8, illustrating the percentage insulin remaining at the end of 60 minutes in the presence of DkOVM).

EDTA and NaGC deaggregate insulin in solution by different mechanisms. EDTA dissociates by chelation of zinc ions and NaGC by hydrophobic micellar incorporation of monomers. Recombinant human insulin is present in solution in various associated forms: monomers, dieters and higher order oligomers (Bai & Chang 1996). When higher order aggregates open up in solution, excess numbers of monomeric units are exposed to α-chymotrypsin mediated degradation. Although the extent of degradation increased at least two-fold in the presence of EDTA and NaGC, the effectiveness of DkOVM did not decrease proportionally. This suggests that the concentration of inhibitors studied should be adjusted based on the associated state of insulin in solution.

Study of Metabolites of α-Chymotrypsin Degradation

The peak areas of metabolite I and metabolite II generated in the presence of α-chymotrypsin were monitored in the presence and absence of DkOVM only. The formation of metabolites I and II was absent in the presence of DkOVM (FIG. 8, illustrating peak areas of Metabolite I and Metabolite II in the absence of DkOVM and in the presence of DkOVM. Values represent the average of at least three independent experiments. Additional metabolites were not detected within one hour at enzyme inhibitor ratios 1:0.5 and 1:1. Four metabolites (A, B, C & D) of α-chymotrypsin mediated degradation of insulin have been characterized (Schilling & Mitra 1991). Comparing the relative proportions of peak areas of metabolites I and II, it appears that they are similar to metabolites D and C as reported.

TABLE 8

| Various Combinations | % I_{60 min} |
|---|---|
| Control (insulin alone) | 100 |
| Chymotrypsin (0.1 μM) | 11.8 ± 1.98 |
| Chymotrypsin + DkOVM 1:0.5 | 52.64 ± 1.43 |
| Chymotrypsin + DkOVM 1:1 | 75.59 ± 1.40 |
| Chymotrypsin + DkOVM 1:2 | 98.77 ± 2.33 |
| Chymotrypsin + Aprotinin 1:1 | 69.65 ± 6.04 |
| Trypsin (0.5 μM) | 68.79 ± 1.85 |
| Trypsin + DkOVM 1:0.1 | 90.35 ± 2.67 |
| Trypsin + DkOVM 1:1 | 99.59 ± 6.16 |
| Trypsin + Aprotinin 1:1 | 97.99 ± 0.08 |
| Chymotrypsin + 0.5 mM EDTA | 0.32 ± 0.33 |
| Chymotrypsin + 30 mM NaGC | 4.66 ± 0.8 |
| Chymotrypsin + 0.5 mM EDTA + DkOVM 1:1 | 57.89 ± 1.52 |
| Chymotrypsin + 30 mM NaGC + DkOVM 1:1 | 56.58 ± 4.84 |

TABLE 9

| Various Combinations | % I_{60 min} |
|---|---|
| Control (insulin alone) | 100 |
| Chymotrypsin (0.1 μM) | 11.8 ± 1.98 |
| Chymotrypsin + CkOVM 1:2 | 9.51 ± 0.25 |
| Chymotrypsin + CkOVM 1:4 | 9.47 ± 0.27 |
| Trypsin (0.5 μM) | 68.79 ± 1.85 |
| Trypsin + CkOVM 1:0.25 | 99.64 ± 2.86 |
| Trypsin + CkOVM 1:0.5 | 96.51 ± 2.79 |
| Trypsin + CkOVM 1:1 | 92.93 ± 3.39 |

### EXAMPLE 2

### Insulin Transport Across Rat Intestine in the Presence of Chicken and Duck Ovomucoid

Analytical Methodology

Radioimmunoassay of Insulin

Insulin radioimmunoassay was performed with a kit supplied by a commercial manufacturer. The range of the standards supplied with the kit include 2 μU/mL-400 μU/mL).

Assay of ³H Testosterone and ¹⁴C Mannitol

The concentration of stock solution of ³H testosterone and ¹⁴C mannitol from the supplier were 0.1 mCi/mL and 0.95 mCi/mL. A working stock was made in Kreb's Bicarbonate buffer to achieve final concentrations of 0.2 μCi/mL for ¹⁴C mannitol and 1 μCi/mL for ³H testosterone. Standard solutions of 5 mL were made from this stock for the concentration range of 0.0004-0.064 μCi/mL for ¹⁴C mannitol and 0.002-0.32 μCi/mL for 3H testosterone. The samples were analyzed in the scintillation counter.

Gradient HPLC Method for the Analysis of Insulin

Stock solution of insulin was prepared by dissolving insulin powder in 0.01N HCl to obtain a final concentration of 100 IU/mL. Diluted concentrations were made with 1% v/v TFA/pH 6.8 buffer using the stock solution with the range 0.05 IU/mL-1 IU/mL. Chromatography was performed under the conditions listed in Table 10 (illustrating the analysis conditions for gradient HPLC method for the analysis of insulin).

TABLE 10

| | |
|---|---|
| Column | Vydac 218MS54 C₁₈ (250 × 4.6 m.m) 5μ 300 Å |
| Mobile phase | (A) Water )0.05% v/v TFA), (B) Acetonitrile (0.05% |
| | v/v TFA) |
| Gradient | 27% B for 5 min, 27-36% in the next 10 min, |
| conditions | 36% B-27% B in the next 1 min, |
| | equilibrate at 27% B for 4 min. |
| Flow rate | 1 mL/min |
| Detection | 210 nm |
| wavelength | |
| AUFS | 0.001 |
| Injection volume | 100 μL |

Isolation of Rat Intestinal Segments

Male Sprague Dawley rats weighing between 200-300 g were used for the permeability experiments. The intestine was excised and the jejunum was isolated by a reported method (Asada et al. 1995). Briefly, the duodenal and ileal segments were removed from top and bottom (13 cm on either side) and the residual intestine was designated as jejunum. The respective segments were mounted in a Navicyte Side-By-Side diffusion apparatus with accessories such as water circulator, flowmeter and humidifier.

The segments were mounted without stripping on a preheated acrylic half-cell and the cell assembly was then placed in a heated block after joining the other half-cell. The exposed surface area was 1.78 cm² and the reservoir volume was 6 mL. The donor and receiver compartments were immediately filled with pre-warmed oxygenated Krebs bicarbonate buffer adjusted to pH 7.4 with NaOH or HCl. The composition of buffer used is shown in Table 11, which illustrates the composition of stock solutions used for preparation of Krebs Bicarbonate solution. The stock solutions can be made in 100 mL or 1 L volume and stored in a refrigerator for one month. For preparation of working buffer, 50 mL of each stock solution above is added to a 1 L volumetric flask containing 750 mL of water. The pH was adjusted to 7.4 when necessary.

TABLE 11

| Stock Solution Components | Molecular Weight | Grams/100 mL added |
|---|---|---|
| Stock A | | |
| Sodium Chloride (NaCl) | 58.4 | 13.82 |
| Potassium Chloride (KCI) | 74.6 | 0.746 |
| Stock B | | |
| Calcium Chloride (CaCl₂) | 147.02 | 0.352 |
| Magnesium Chloride | 203.3 | 0.488 |
| (MgCl₂ 6H₂O) | | |
| Stock C | | |
| Sodium Phosphate | 137.99 | 0.1104 |
| (NaH₂PO₄H₂O) | | |
| Sodium Phosphate | 142.0 | 0.454 |
| (Na₂HPO₄) | | |
| Stock D | | |
| Sodium Bicarbonate | 84.0 | 4.2 |
| (NaHCO₃) | | |

The mucosal buffer consisted of 40 mM mannitol and the serosal buffer consisted of 40 mM glucose. Both donor and receiver media had an osmotic pressure of 290-300 mOsm/kg that was verified with an Osmometer. Mannitol equalizes the osmotic load between the mucosal and apical buffers and glucose helps to maintain tissue viability. The buffer was circulated by a gas lift (95%O₂/5%CO₂). The flow rate of gas lift was adjusted to 10±2 mL/min using a flow meter. The tissues were equilibrated for 10 min before the drug solution was added.

Transport Studies of Insulin

Stock solutions of insulin, inhibitors and enzyme were prepared in mucosal buffer. After equilibration for 10 min, drug and inhibitor solution was added on the mucosal side so that the final concentration of insulin was 100 μM and that of inhibitors was between 0-1.5 μM. For the marker studies, the concentration of ¹⁴C mannitol in donor compartment was 3.5×10⁻⁵ μM and that of H testosterone was 4×10⁻² μM. The integrity of the tissues was determined by calculating the permeability coefficient (Pₐₚₚ) of mannitol. For α-chymotrypsin studies, the enzyme was added immediately after the addition of drug and inhibitor solution to achieve a final concentration of 0.5 μM. Samples (1 mL) were taken from the serosal side at various times up to 180 min and replaced with fresh transport medium. Aliquots of 10 μL were taken from the mucosal side at the beginning and end of the experiment and analyzed by a HPLC method discussed later. Receiver compartment insulin was analyzed using a solid phase radioimmunoassay. The logit-log graph of percent bound vs. concentration was used to interpolate the values of unknown concentrations. The radioactive samples were analyzed by using a liquid scintillation counter.

Stability Studies in the Presence of Duck Ovomucoid

Insulin solutions at a concentration of 100 μM were incubated at 37° C. in Krebs bicarbonate buffer. The degradation profiles were generated in the presence of 0.5 μM α-chymotrypsin over a period of 3 hours that served as controls. DkOVM was evaluated for its efficiency against enzyme mediated degradation of insulin at 1:1 and 1:2 ratio of enzyme to inhibitor. Stability studies were also carried out by incubating insulin with DkOVM in the absence of α-chymotrypsin. Insulin and enzyme solutions were incubated for 15 min at 37° C. prior to starting the experiments. Samples were taken up to 180 min and immediately diluted with cold 1% TFA/TRIS to reduce the pH to 2.5. The samples were analyzed by the RP-HPLC method reported earlier.

Data Analysis

Apparent permeability coefficients (Pₐₚₚ) of insulin, D-[1-¹⁴C] mannitol and (7-³H] testosterone in the presence and absence of CkOVM, DkOVM and α-chymotrypsin were calculated using the following equation: Pₐₚₚ=(1/AC₀) (dM/dt), where dM/dt is the flux across the intestinal membrane (m. IU/min or μCi/min), A is the surface area of the membrane (1.78 cm 2) and C₀ is the initial drug concentration (100 μM). Flux was determined from the slope of the cumulative amount permeated vs. time plot. For the stability studies, amount of insulin remaining vs. time (%) was plotted for a period of three hours. The results of experiments performed at least in triplicate are presented as mean±SE. Statistical differences between permeability in the presence of DkOVM and CkOVM and the means were determined by one-way analysis of variance (ANOVA). The criterion for statistical significance was p<0.05.

Results and Discussion of the Transport of Insulin in the Presence of Chicken and Duck Ovomucoid

Analytical Methodology

Radioimmunoassay of Insulin

The range of standard curve was different with each kit that was supplied by the manufacturer. For each kit a standard curve was constructed by plotting log (conc) on the X axis and Logit (Y) on the Y axis. Logit Y was calculated by using the expression: LogitY=log(1/1−y). The counts generated by a kit for analysis of insulin is shown in Table 12 (Counts per Minute (CPM) values generated for a radioimmunoassay of insulin). A plot of log (cond) bound vs. Logit Y for this kit revealed a slope value of −1.691176 and an intercept value of 2.37. The correlation coefficient obtained for this run was 0.9977.

TABLE 12

| Tube | Conc | CPM1 | CPM2 | Average | Net CPM | % Bound | Log (Conc) | Logit Y |
|---|---|---|---|---|---|---|---|---|
| T | | 33488 | 33629 | 33558.5 | 32837 | | | |
| NSB | | 638 | 625 | 631.5 | | | | |
| A(MB) | 0 | 14278 | 14273 | 14275.5 | 13644 | 100 | | |
| B | 6.6 | 10601 | 10832 | 10716.5 | 10085 | 73.9 | 0.82 | 1.04 |
| C | 19 | 7971 | 7692 | 7831.5 | 7200 | 52.8 | 1.28 | 0.112 |
| D | 58 | 5353 | 5363 | 5358 | 4726.5 | 34.6 | 1.76 | −0.64 |
| E | 105.0 | 4271 | 4237 | 4254 | 3622.5 | 26.6 | 2.02 | −1.02 |
| F | 200 | 3116 | 3098 | 3098 | 2466.5 | 18.1 | 2.3 | −1.51 |
| G | 400 | 2226 | 2205 | 2205 | 1573.5 | 11.5 | 2.6 | −2.1 |

Assay of ³H Testosterone and ¹⁴C Mannitol

The proportion of isotopes used for generation of standard curve of ³H testosterone and ¹⁴C mannitol was the same as used during the experiment. The DPM values were averaged for the concentration studied. A plot of concentration on the X axis and DPM on the Y axis was constructed to get the value of slope and intercept. The values of slope and intercept were used to estimate the unknown concentrations in the permeability experiments. For the assay of ³H testosterone, the DPM values obtained for the concentration range are shown in Table 13. For this range of concentration a slope and intercept value of 1359769 and −941.29 was obtained with square of correlation coefficient 0.999. The DPM values obtained for standard concentrations of ¹⁴C mannitol are shown in Table 14. For the concentration range studied, a slope and intercept value of 1642397 and 4.88 with square of correlation coefficient 0.999 was obtained.

TABLE 13

| Conc | | | | | |
|---|---|---|---|---|---|
| (μCi/mL) | DPM-1 | DPM-2 | DMP-3 | Average | RSD |
| 0.0005 | 558.26 | 524.8 | 503.41 | 528.8233 | 5.23 |
| 0.001 | 1067.47 | 1064.76 | 1066.14 | 1066.123 | 0.13 |
| 0.002 | 2108.32 | 2174.02 | 2174.19 | 2152.177 | 1.76 |
| 0.004 | 4466.87 | 4528.48 | 4546.1 | 4513.817 | 0.92 |
| 0.008 | 9317.3 | 9503.85 | 9487.7 | 9436.283 | 1.10 |
| 0.016 | 19782.36 | 19801.27 | 19851.32 | 19811.65 | 0.18 |
| 0.032 | 41065.19 | 41327.89 | 41679.11 | 41357.4 | 0.74 |
| 0.064 | 86032.34 | 87372.36 | 87528.52 | 86977.74 | 0.95 |

TABLE 14

| Conc | | | | | |
|---|---|---|---|---|---|
| (μCi/mL) | DPM-1 | DPM-2 | DMP-3 | Average | RSD |
| 0.0001875 | 334.3 | 320.36 | 342.13 | 332.26 | 3.32 |
| 0.000375 | 634.19 | 620.85 | 629.13 | 628.06 | 1.07 |
| 0.00075 | 1227.66 | 1235.52 | 1235.66 | 1232.95 | 0.37 |
| 0.0015 | 2437.35 | 2486.25 | 2463.87 | 2462.49 | 0.99 |
| 0.003 | 4948.11 | 4957.99 | 4941.51 | 4949.20 | 0.17 |
| 0.006 | 9896.65 | 9870.22 | 9796.5 | 9854.46 | 0.53 |
| 0.012 | 19592.5 | 19691.09 | 19685.64 | 19656.41 | 0.28 |
| 0.024 | 39359.62 | 39446.22 | 39545.07 | 39450.30 | 0.24 |

Gradient HPLC Method for the Analysis of Insulin

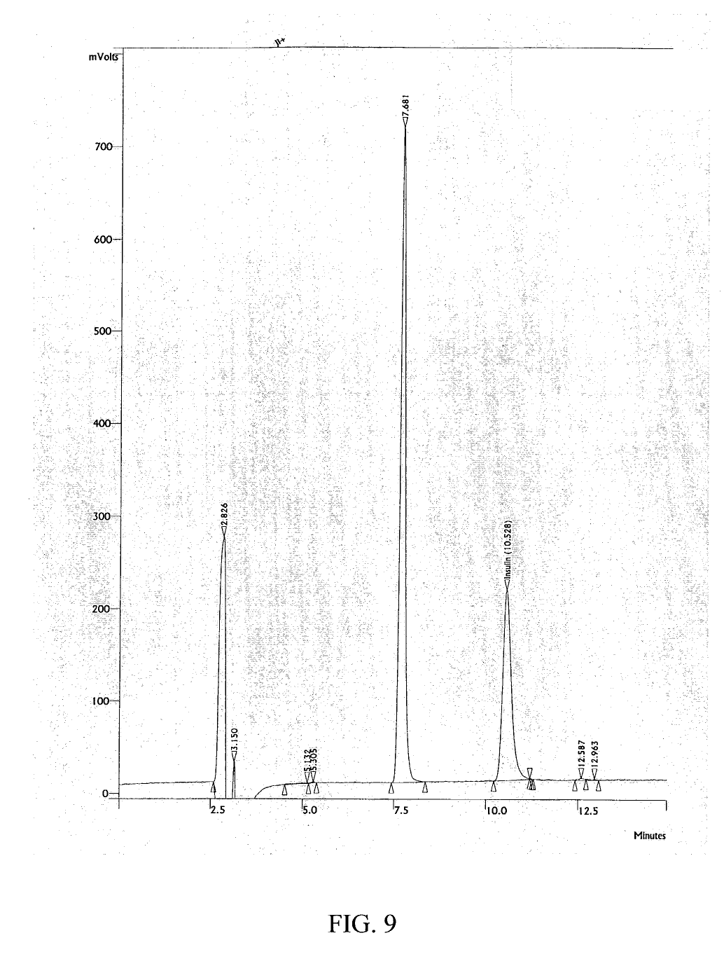

The mobile phase conditions selected were suitable for separation of insulin from the preservative m-cresol used in commercial formulation as shown in FIG. 9 (representative chromatogram of m-cresol and insulin). From the chromatogram it can be seen that m-cresol eluting at 7.68 min and insulin eluting at 10.52 min are well separated. The range of standard curve for analysis was between 0.05 IU/mL-1.0 IU/mL. For this range the slope value of a typical run was 3150330 and the intercept value was −187569. The correlation coefficient (r²) for this run was 0.998. A standard curve was constructed on each day unknown samples were analyzed and estimations were done based on slope and intercept of this run.

Influence of DkOVM and CkOVM on the Permeability of Insulin

The cumulative amount of insulin permeated at the end of three hours from various segments of the intestine is shown in FIG. 10 (cumulative amount of insulin released from various segments of the intestine at the end of three hours, values represent the average of at least three experiments). The cumulative amount of insulin released was 0.031±0.022 IU from the duodenum, 0.04±0.023 IU from the jejunum and 0.052±0.021 IU from the ileum. This shows that the permeability is comparable from jejunum and ileum but greater than duodenum. These results are comparable with studies in the literature with respect to permeability of insulin from various segments of the rat intestine (Asada et al. 1995). For analysis of samples from various segments of the intestine, an HPLC method was used. The method could not detect samples earlier than 3 hours. For this reason, cumulative amount of drug released at the end of 3 hours is reported. For further studies a more sensitive radioimmunoassay method was used.

Based on data from permeability of insulin from various segments of the intestine, the jejunum segment was selected for further studies. Further, jejunum is the longest as well as the largest section of the small intestine through which absorption occurs. The concentrations of insulin and inhibitors selected were comparable to the ratio used in an earlier stability study (Agarwal et al. 2000). CkOVM and DkOVM purity was greater than 90% and molecular weights of 27 kD and 28 kD respectively were used for all calculations (Rhodes et al. 1960).

Measurement of mannitol permeability is a convenient and relatively sensitive measure of the integrity and permeability of the intestinal layer (Marks et al. 1991). The measurements reflect the resistance across the tight junctions and not the cell membrane. Table 15 lists the permeability coefficients of insulin in the presence of CkOVM, DkOVM and α-chymotrypsin at various concentrations. The Pₐₚₚ value calculated from the data shown in Table 15 was found to be 3.465±0.251×10⁻⁶ cm/sec. This value is in agreement with values reported using the same apparatus, (Grass & Sweetana 1988), suggesting that the integrity of the tissue was maintained over the duration of the studies. The Pₐₚₚ calculated for insulin under identical conditions of mannitol studies reported above was 0.922±0.168×10⁻⁷ cm/sec (Table 15). This value is less than the reported values of 5±2×10⁻⁷ cm/sec (Schilling & Mitra 1990) and 12.27±1.73×10⁻⁷ cm/sec (Asada et al. 1995) for Pₐₚₚ of insulin from rat jejunum.

A recent study estimated the apical epithelial permeability of insulin to be 0.32×10⁻⁷ cm/sec (Stoll et al. 2000). However, the value obtained in our laboratory is used as reference for the evaluation of permeability of insulin in the presence of ovomucoids. The variations in permeability coefficients may be attributed to differences in apparatii, tissue preparation, concentrations studied, analytical method employed, and the duration of study. The permeability of insulin decreased in the presence of both the inhibitors. The results are shown in FIG. 11 and Table 15. FIG. 11 illustrates the cumulative amount of insulin permeated (m.IU) vs. time in the absence of DkOVM and at DkOVM concentrations 0.5 μM, 1.0 μM, and 1.5 μM respectively.

The Pₐₚₚ of insulin in the presence of DkOVM decreased in a concentration dependent manner (FIG. 10). At 1.5 μM concentration of DkOVM the Pₐₚₚ was 0.066±0.043×10⁻⁷ cm/sec, representing a substantial decrease when compared to the control value for insulin (Table 15). The corresponding permeability ratio has actually decreased from 0.55 at 0.5 μM DkOVM in insulin solution to 0.07 with 1.5 μM DkOVM in insulin solution. Similarly, the permeability ratio decreased from 0.22 to 0.02 when the CkOVM concentration in insulin solution increased from 0.5 to 1.5 μM (Table 15). The present study indicates that there are differences in permeability of insulin with the type of ovomucoid used (DkOVM vs. CkOVM). It has been reported that insulin is absorbed transcellularly from enterocytes using an immunohistochemical method (Bendayan et al. 1994). The uptake of insulin from hepatocytes and adipocytes is by a receptor-mediated process (Sonne 1988). The location of receptors for insulin at the enterocyte level has been established (Bergeron et al. 1980; Pillion et al. 1985; Gingerich et al. 1987). Reports suggest the presence of insulin receptor on enterocytes both at the apical side and basolateral side. A recent report suggests the presence of insulin receptor on the apical side in the small intestine region in rats (Saffran et al. 1997).

The decrease in permeability of insulin in the presence of ovomucoids may be explained in part by the action of insulin on adipocytes. It is hypothesized that binding of insulin to the adipocyte plasma membrane activates a membrane protease that results in the formation of a soluble factor that stimulates pyruvate dehydrogenase activity (Seals & Czech 1980; Czech et al. 1984; Seals & Czech 1982; Seals & Czech 1981). This activation is blocked by trypsin-like proteases such as ovomucoid and soybean trypsin inhibitor by preventing the interaction of the protease and its endogeneous membrane substrate. If a similar event happens at the junction of enterocyte cells, insulin will not be able to bind to its receptors and get transported. This might be the reason for reduced permeability of insulin at the enterocyte level. The reduction in permeability could also be due to mechanical obstruction of insulin (MW 6 KDa) by a much larger molecule (CkOVM 27 kD and DkOVM 28 kD).

TABLE 15

| | Permeability | |
|---|---|---|
| Solutions in donor compartment | (P × 10⁷ cm/sec) | Ratio |
| Insulin 100 μM | 0.922 ± 0.168 | 1.0 |
| Insulin 100 μM + | 0.491 ± 0.022 | 0.54 |
| DkOVM 0.5 μM | | |
| DkOVM 1.0 μM | 0.321 ± 0.089 | 0.35 |
| DkOVM 1.5 μM | 0.066 ± 0.043 | 0.07 |
| Insulin 100 μM + | 0.206 ± 0.047 | 0.22 |
| CkOVM 05 μM | | |
| CkOVM 1.0 μM | 0.291 ± 0.095 | 0.32 |
| CkOVM 1.5 μM | 0.018 ± 0.008 | 0.02 |
| Insulin 100 μM + a-chymotrypsin 0.5 μM + | 0.0 | |
| DkOVM 0.0 μM | | |
| DkOVM 0.5 μM | 0.170 ± 0.046 | 1.0 |
| DkOVM 1.0 μM | 0.333 ± 0.057 | 1.96 |

Influence of DkOVM and CkOVM on Permeability of Mannitol and Testosterone

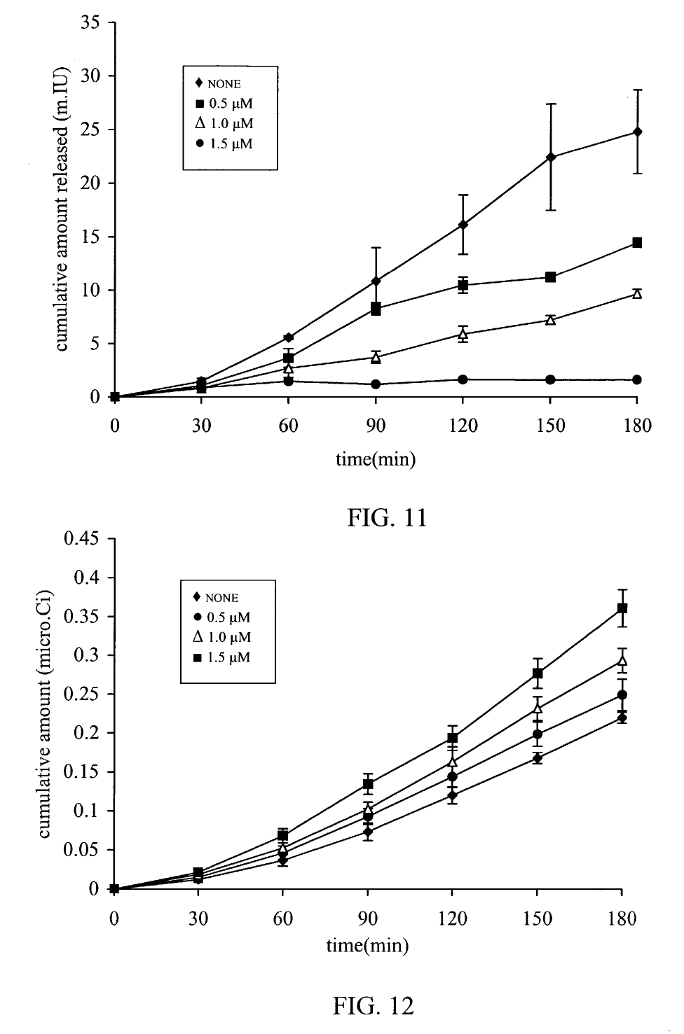

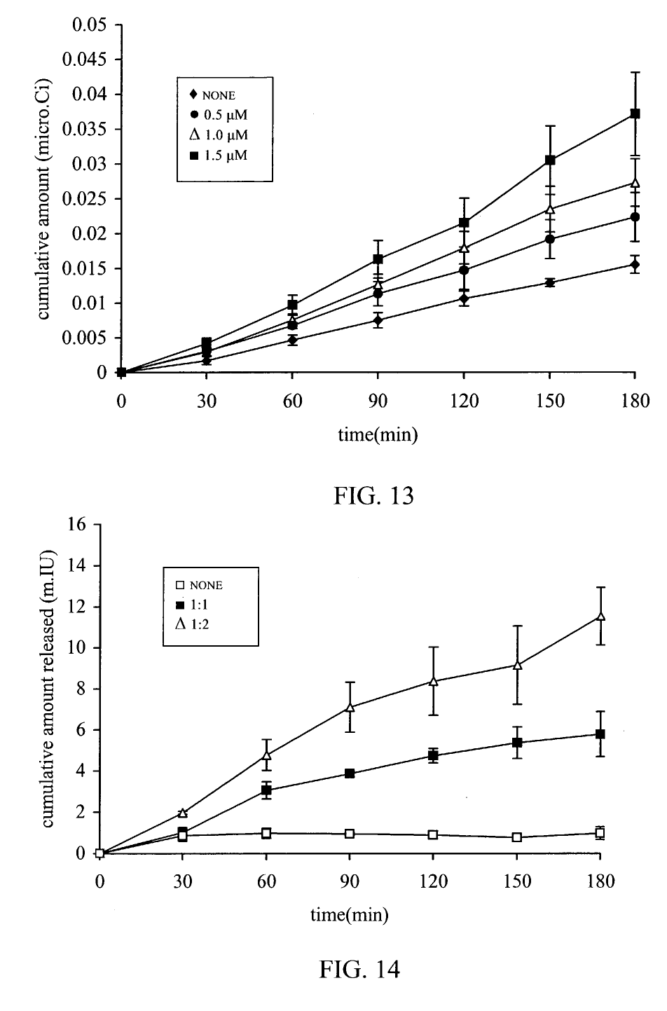

Parameters such as TEER and permeability of progesterone, testosterone, mannitol and phenol red have been used to assess the damage potential of absorption modifiers on cells. The influence of CkOVM and DkOVM on the integrity of cell membrane and junctional integrity was evaluated by studying the permeability of a lipophilic and hydrophilic marker. The permeability of testosterone, a lipophilic marker, increased in the presence of DkOVM in a concentration dependent manner (FIG. 12). FIG. 12 illustrates the cumulative amount (μ.Ci) of [7-³H] testosterone permeated vs. time in the presence of DkOVM at concentrations 0.5 μM, 1.0 μM and 1.5 μM respectively. The Pₐₚₚ value increased by 1.61 fold and 1.34 fold in the presence of DkOVM and CkOVM at 1.5 μM concentration (Table 16, listing the permeability coefficients of [7-³H] testosterone and D⁻[¹-¹⁴C] mannitol in the presence of DkOVM and CkOVM. The permeability of mannitol, a hydrophilic marker, also increased in the presence of DkOVM in a concentration dependent manner (FIG. 13). FIG. 13 illustrates the cumulative amount (μ.Ci) of D-[1-¹⁴C] mannitol permeated vs. time in the presence of DkOVM at concentrations 0.5 μM, 1.0 μM and 1.5 μM respectively. The Pₐₚₚ value of mannitol increased by 2.39 fold and 3.38 fold in the presence of DkOVM and CkOVM at 1.5 μM concentration (Table 16). Differences in permeability of testosterone and mannitol were found to be dependent on the type of ovomucoid used (DkOVM vs. CkOVM).

The increase in permeability of a lipophilic and hydrophilic marker indicates that ovomucoids bring about changes in the epithelial cells in a concentration dependent manner. The changes may be due to decrease in the fluidity of the cell membrane and tight junctional integrity between the cells. This causes the permeability of testosterone and mannitol to increase when compared to control values. The increase in permeability coefficients of testosterone and mannitol may be explained by the role of lectin type binding to the mucosal surfaces. Damage to cells have been observed due to binding of lectins to the bound sugars on the cell membrane. Lectins present in wheat germ agglutinin bound to the sugar molecules on surface of cells and cause damage to the cells (Lorenzsonn & Olsen 1982; Sjolander et al. 1986). The kidney bean lectin effects the function of the entire gastrointestinal tract (Bardocz et al. 1995). Ovomucoids have a glycoprotein portion that is assumed to interact with the natural lectins on the mucosal surface of the intestine (Valuev et al. 1999). This binding may also initiate damage to the cells as does the process of lectin binding to natural sugars.

Insulin Stability and Permeability in the Presence of α-chymotrypsin and DkOVM

Insulin is likely to encounter the degrading effect of luminal enzymes during absorption in vivo. During the preparation of tissues for mounting on the diffusion chamber, the enzymes were washed off. To simulate the scenario of absorption in the presence of enzymes, permeability of insulin was also evaluated in the presence of α-chymotrypsin and DkOVM. Stability studies were performed with DkOVM only as it inhibits both trypsin and α-chymotrypsin mediated degradation of insulin while CkOVM inhibits only trypsin mediated degradation of insulin (Agarwal et al. 2000). The permeability of insulin in the presence of α-chymotrypsin was found to be negligible as shown in FIG. 14, illustrating the cumulative amount of insulin permeated (m.IU) vs. time in the presence of α-chymotrypsin in the absence and presence of DkOVM at 1:1 and 1:2 ratio of enzyme to inhibitor. This was expected since there are several reports of insulin degradation with this enzyme. However, the permeability was found to increase as a function of increasing concentration of DkOVM (FIG. 14). At 1:2 ratio of enzyme to inhibitor, the Pₐₚₚ of insulin in the presence of α-chymotrypsin was 0.333±0.057×10⁻⁷ cm/sec (Table 15). This represents a 2-fold increase in permeability when compared to the insulin permeability from a solution containing 1:1 ratio of enzyme to inhibitor (Table 15) and a significant increase in permeability when compared to the control value.

TABLE 16
Permeability coefficients of [7-³H] testosterone and
D-[1-¹⁴C] mannitol in the presence of DkOVM and CkOVM

| Solutions in | ³H testosterone | | ¹⁴C Mannitol | |
|---|---|---|---|---|
| donor | Permeability × | | Permeability × | |
| compartment | 10⁶ cm/sec | Ratio | 10⁶ cm/sec | Ratio |
| Control | 19.750 ± 0.310 | 1.0 | 3.465 ± 0.251 | 1.0 |
| DkOVM 0.5 μM | 23.354 ± 1.955^{a} | 1.18 | 5.702 ± 0.7643^{a} | 1.65 |
| DkOVM 1.0 μM | 26.015 ± 1.341^{b} | 1.31 | 5.958 ± 0.944^{a} | 1.72 |
| DkOVM 1.5 μM | 31.858 ± 1.897^{b} | 1.61 | 8.278 ± 1.321^{b} | 2.39 |
| Control | 19.750 ± 0.310 | 1.0 | 3.465 ± 0.251 | 1.0 |
| CkOVM 0.5 μM | 21.667 ± 0672^{a} | 1.1 | 4.495 ± 0.784^{a} | 1.30 |
| CkOVM 1.0 μM | 23.256 ± 2.623^{a} | 1.18 | 6.320 ± 1.100^{b} | 1.83 |
| CkOVM 1.5 μM | 26.453 ± 2.278^{b} | 1.34 | 11.715 ± 0.284^{b} | 3.38 |

^{a}Not significantly different
^{b}Significantly different

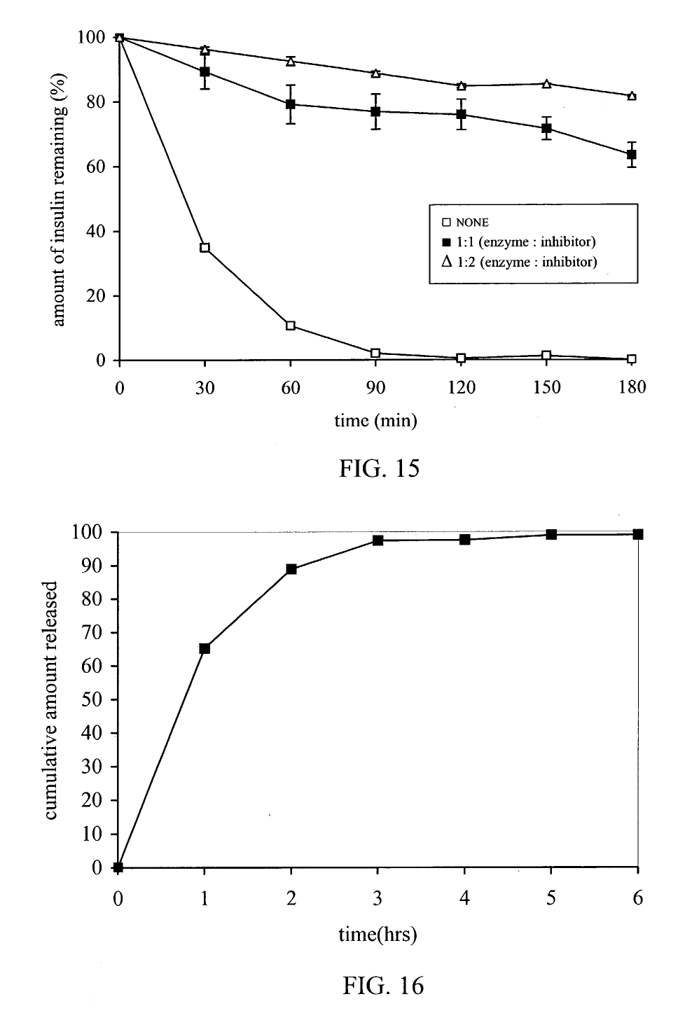

Under conditions simulating the donor compartment concentrations with 1:1 and 1:2 ratio of enzyme to inhibitor, insulin remaining (%) at the end of 3 hours was 63.34±3.83 and 81.53±0.34 respectively (FIG. 15). FIG. 15 illustrates the chymotrypsin-mediated degradation of insulin as a function of time in the absence of DkOVM and at enzyme-to-inhibitor ratios of 1:1 and 1:2. Assuming a linear degradation rate of insulin at 1:2 ratio of enzyme to inhibitor, the rate of degradation was 6.66%/hr. This was 2 times lower when compared to rate of degradation of insulin at 1:1 ratio (12.22%/hr). This 2-fold reduction in degradation of insulin could explain the 2-fold enhancement of its permeability.

In the presence of α-chymotrypsin and DkOVM, the events that are occurring simultaneously include enzyme mediated insulin degradation and permeability of insulin. In the absence of DkOVM, there is extensive degradation of insulin. This is evident from the negligible value of insulin (%) remaining at the end of 3 hours in the stability experiments. When DkOVM is added it binds to α-chymotrypsin and slows the degradation of insulin. Consequently, the permeability of insulin increases due to the increased amount of insulin in donor compartment in the presence of enzyme inhibitor.

Ovomucoids represent attractive absorption modifiers for the oral delivery of proteins. This is due to their inhibitory action towards enzymes present in the gut and binding to the natural lectins on the mucosal cells through their carbohydrate moeity. In the present investigation it was found that ovomucoids decreased the permeability of insulin, increased the permeability of a hydrophilic and lipophilic marker and increased the permeability of insulin in the presence of α-chymotrypsin.

The decrease in permeability of insulin in the presence of ovomucoids was unexpected. The steric hindrance of insulin during transport is also possible by the large ovomucoid molecule. Such steric hindrance was not observed when mannitol and testosterone were used as markers. Also, increased transport of markers excludes the contribution of transcellular and paracellular route in the transport of insulin. The decrease in permeability of insulin may not effect the absorption in vivo to a significant extent. Insulin has been reported frequently to encounter the action of enzymes such as trypsin and α-chymotrypsin during the absorption process in vivo. The present study has demonstrated that the permeability of insulin is enhanced in the presence of α-chymotrypsin and DkOVM. Therefore, it would be appropriate to prepare an oral dosage form of insulin with duck ovomucoid.

### EXAMPLE 3

### Microencapsulation of Insulin and In Vitro Dissolution Stability in the Presence of Enzymes

HPLC Analysis of Insulin

HPLC analysis of insulin was performed according to the methodology previously described above.

Microencapsulation by Coprecipitation

Insulin was dissolved in 0.01 N HCl at a concentration of 100 IU/ml. 8 ml of this solution was added to a 17 ml of alcohol USP contained in a beaker under stirring using a magnetic stirrer rotating at 400 rpm. To this solution, 2 gm of Eudragit was added over a period of 10 mins. The polymeric solution was allowed to stir for an additional 5 min to allow the polymer to dissolve completely. The solution was then transferred by a peristaltic pump from a fixed height to a beaker containing cold water (4° C.) with stirring by a homogenizer set at 10 k rpm. Stirring was continued for an additional minute after the polymeric solution containing the drug was completely transferred. The suspended microcapsules were separated from the liquid by filtration under vacuum using a Whatman #4 filter paper. The microcapsules were transferred to a porcelain dish and allowed to dry overnight in an oven set at 40° C. The microcapsules were passed through sieve #40 and retained on sieve #120. Aggregates of microcapsules were milled in a pestle and mortar before passing through the sieves. The microcapsules were then weighed and transferred to a screw-capped scintillation vial for further use.

Addition of Salts in the Precipitating Medium

Salts such as calcium chloride and sodium sulfate, and a surfactant, such as Tween 80 were added in cold water during precipitation at a concentration of 0.25% w/v.

Ratio of Polymeric Solution to Volume of Precipitating Medium

The ratio of polymeric solution to volume of precipitating medium used was 1:2, 1:4, and 1:6.

Drug Encapsulation Efficiency

25 mg of microcapsules were added to 5 ml of alcohol USP contained in a scintillation vial. After the microcapsules dissolved completely, 5 ml of phosphate buffer pH 6.8 was added to this solution. From this solution, 0.5 ml was transferred HPLC vial, diluted to 1 ml with phosphate buffer added and analyzed by the HPLC method reported earlier.

Treatment with 0.1N HCl

25 mg of microcapsules were soaked in 1 ml of 0.1 N HCl that was equilibrated at 37° C. in a water bath. After the microcapsules were immersed for 2 hours a sample was taken and analyzed by the HPLC method reported earlier.

Dissolution Studies

Dissolution studies were performed in a dissolution apparatus fitted with a 100 ml conversion kit. Phosphate buffer USP pH 6.8 was used as the dissolution media. The buffer was prepared by mixing 75% of 0.2 M tribasic sodium phosphate and 25% of 0.1 N HCl. The pH was adjusted to 6.8 with 2 M NaOH or 2 M HCl. The temperature of dissolution media was 37° C. and the rotation speed of the paddles was set to 50 rpm. Microcapsules (125 mg) equivalent to 50 IU of insulin were filled in a size 00 capsule and transferred to the prewarmed dissolution medium. Samples (2 ml) were withdrawn every hour upto 6 hours and the volume was replaced immediately by fresh phosphate buffer. Samples were analyzed by the HPLC method reported earlier.

Dissolution Stability in the Presence of Enzymes

The dissolution set up was the same as above except for the following modifications. The capsule contained microcapsules of insulin with various amounts of chicken and duck ovomucoid. The dissolution medium contained 0.5 μM trypsin for capsules containing CkOVM and 0.1 μM α-chymotrypsin for capsules containing DkOVM. Samples withdrawn (2 mL) were immediately treated with cold 1% v/v TFA/pH 6.8 buffer (2 mL) to stop the enzymatic activity. The samples were maintained at 8° C. in the autosampler throughout the duration of the analysis.

Results and Discussion of Microencapsulation of Insulin and In Vitro Dissolution Stability in the Presence Of Enzymes

Microencapsulation by Coprecipitation

The coprecipitation technique involves dissolving the polymer and drug in an organic solvent and then adding a non-solvent to precipitate the drug and polymer. During the precipitation process, the polymer particles encapsulate the drug. Coprecipitates have been successfully prepared for drug polymer combinations of ibuprofen/Eudragit S 100 (Khan et al. 1995), indomethacin/mixture of Eudragit RS 100 and RL 100 (Kamachi et al. 1995b) and Ketprofen and Eudragit S 100 (Khan et al. 1996). In these studies, alcohol was used as a solvent for dissolving the polymer and drug. Cold water was used as a non-solvent for precipitation. The drug to polymer ratio used was as high as 10:1 and the particle size obtained was less than 800 μM.

The approximate range of intestinal transit of a dosage form in the small intestine is between 3-6 hours (Davis et al. 1986). Our aim was to evaluate this technique for preparing microcapsules of insulin at low drug concentration that could release the drug over a period of 6 hours. Preliminary experiments were performed to determine the effect of various formulation and process factors mentioned before. These factors were evaluated with respect to percentage yield of microcapsules, drug encapsulation efficiency and dissolution studies.

Based on preliminary experiments, it was determined that insulin could remain in solution with the polymer in 32% v/v mixtures of 0.01 N HCl and alcohol. Below 32% 0.01N HCl, insulin is in the form of suspension in the polymeric solution. Microcapsules prepared without the use of 0.01 N HCl were low in encapsulation efficiency and had variability in drug content. This was evident from high concentrations of insulin in the filtrate. Subsequently, the proportion of 0.01 N HCl in alcohol was fixed at 50% v/v. Microcapsules with Eudragit L100 and Eudragit S100 were obtained with high yield (>80%) but those with Eudragit L100-55 had extremely low yield (<20%). There was evidence of hazy filtrate during the preparation of microcapsules with Eudragit L100-55. Analysis of filtrate revealed high concentrations of insulin.

Eudragit L100-55 has 0.7% sodium lauryl sulfate and 2.3% polysorbate 80 as emulsifiers (HULS America 1997). The presence of these emulsifiers could have enhanced the solubility of the drug and polymer in the mixture of 50% 0.01 N HCl/alcohol. Consequently, the drug and polymer did not precipitate completely under the conditions used. Based on these studies, Eudragit L100 and S100 were chosen for further studies. The concentration of polymer was fixed at 8% w/v. Insulin concentration with respect to the polymer was 1.39% w/w. Polymer concentrations of 4% w/v and 16% w/v were tried with the same drug loading before deciding on 8% w/v concentration. The lower concentration was difficult to work with due to extremely low yield. The higher concentration was not used due to high variability with respect to drug loading.

The stirring time of 15 minutes was fixed after initial experiments. It is important that the polymer be added slowly over a period of 10 minutes to avoid the formation of clumps. The additional 5 minutes of stirring time helps in the preparation of a clear polymeric solution containing the drug. Initial mixing experiments were done with a homogenizer set at 10 k rpm and a magnetic stirrer at 400 rpm. Comparison of dissolution profiles and drug encapsulation efficiencies did not reveal significant differences. The magnetic stirrer was chosen to avoid exposing the protein to high shear rates involved during mixing in the homogenizer.

The addition of polymeric solution to the cold water was done with the help of a transfer pipette and a peristaltic pump. The rate of addition was not easily controlled with a transfer pipette when compared to the peristaltic pump. Further, it was found that rate of addition had an influence on the drug encapsulation efficiency. Similar results have been obtained earlier for encapsulation efficiency of ibuprofen with Eudragit S 100 (Khan et al. 1994). The peristaltic pump was chosen to achieve strict control on the rate of addition of the polymeric drug solution to water. Precipitation of polymer microcapsules could be achieved by stirring with a homogenizer or a magnetic stirrer. The microcapsules obtained while precipitating with a magnetic stirrer had a tendency to agglomerate and stop the stirrer midway during the precipitation process. This problem could be averted by the use of homogenizer.

A representative dissolution profile of a batch of microcapsules is shown in FIG. 16. This profile was used for comparison purposes to evaluate the effect of variables mentioned in this section. By keeping these operating conditions constant, the effect of other factors such as addition of salts in the precipitating medium and ratio of water to polymeric drug solution could be assessed.

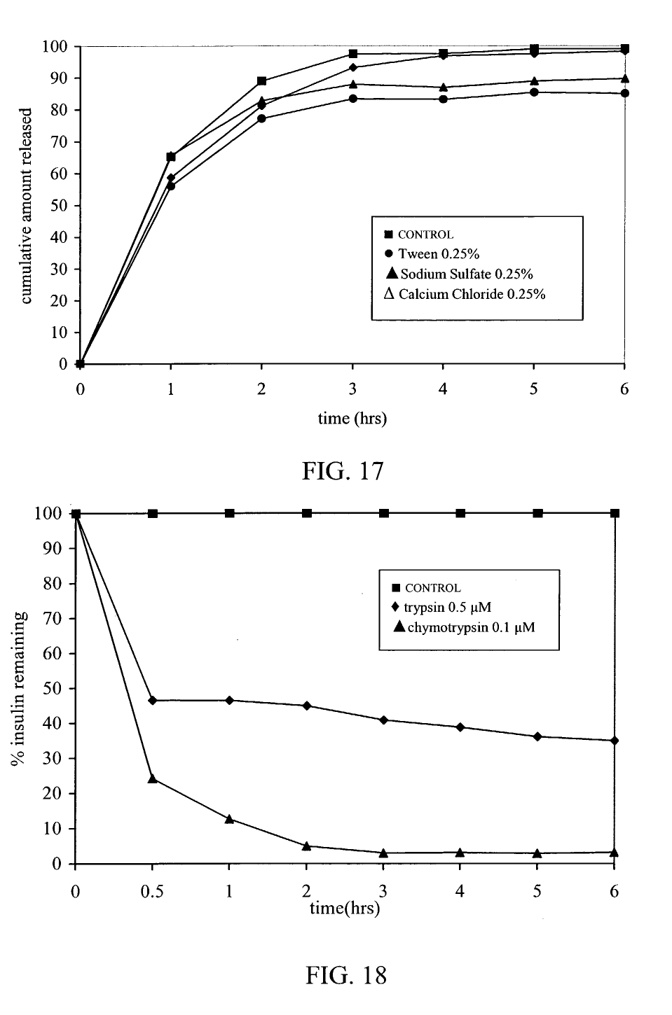

The addition of salts and surfactant in the precipitating medium was attempted to decrease agglomerate formation. Electrolytes such as calcium chloride, sodium sulfate and surfactant such as Tween 80 were used at 0.25% w/v concentration in the precipitating medium during microcapsule formation. Comparison of dissolution profiles is shown in FIG. 17, illustrating the effect of salts in the precipitating medium on the dissolution of insulin microcapsules: control, sodium sulfate 0.25%, calcium chloride 0.25%, Tween 0.25%. As shown in the figure, both calcium chloride and sodium sulfate are slowing the release of insulin to some extent. This may be due to the increased likelihood of partial neutralization of the negative charge of insulin in phosphate buffer which is at a higher pH (6.8) than the pH for the isoelectric point of insulin (5.5). Similar to floculation of charged particles in the presence of oppositely charged electrolytes, the fraction of insulin microcapsules with increased diameter and less overall surface area may be greater. The decreased dissolution in the presence of surfactant, Tween 80, may be due to increased encapsulation efficiency.

The ratios of polymeric drug solution versus volume of precipitating medium investigated in our laboratory were 1:2, 1:4 and 1:6. The drug encapsulation efficiency was effected by the ratio. The range of encapsulation efficiency varied from 91.83%-103.6%. The ratio of water volume versus polymeric drug solution is critical for the formation of microcapsules. Microcapsules can start forming only when the volume of water is more than a critical ratio with respect to the volume of polymeric solution. The ratio established for a 8% ethanolic solution of Eudragit L100 in water for appearance of turbidity was established as 1:1.38 (Kachrimanis et al. 2000).

Encapsulation Efficiency

The yield of microcapsules obtained was close to 80%. The loss may be attributed to the polymeric solution sticking to the glass beaker used in transferring the solution to the precipitating medium. The encapsulation efficiency determined from a triplicate of three runs was 95±2.0%. The proportion of buffer used in alcohol is critical for accurate determination of encapsulation efficiency.

Treatment with 0.1 N HCl

The pretreatment of microcapsules in 0.1 N HCl was necessary to make sure that the acid does not destroy the tablet integrity. If the tablet integrity is lost in the acid medium of the stomach, it would be a poor formulation for release in the jejunum. Insulin was not detectable in the sample analyzed from the supernatant of microcapsules soaked in 0.1 N HCl for two hours at 37° C. This reveals that insulin in not present on the surface of the microcapsules and the microcapsules are enteric in nature.

Dissolution Stability in the Presence of Enzymes

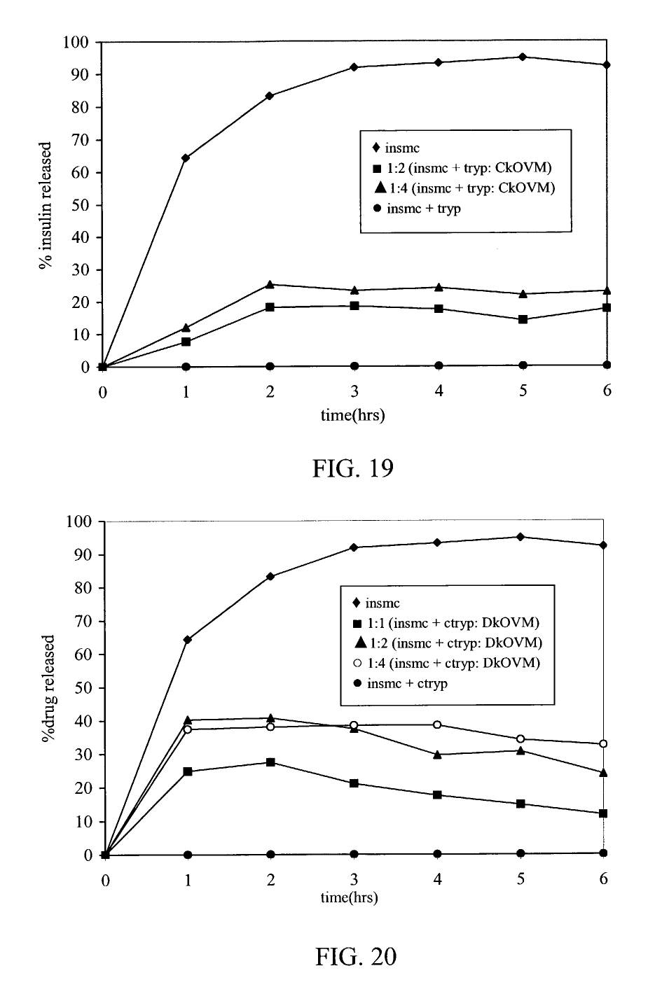

Stability of insulin solution in the presence of trypsin and α-chymotrypsin is shown in FIG. 18 (illustrating the degradation of insulin solution (50 IU) in the presence of trypsin and α-chymotrypsin: control, trypsin 0.5 μM, chymotrypsin 0.1 μM). Dissolution stability of insulin released from microcapsules in the presence of 0.5 μM trypsin and various ratios of CkOVM is shown in FIG. 19 (illustrating insulin dissolution stability in the presence of trypsin and CkOVM: insmc, insmc+tryp: CkOVM 1:2, ins+tryp: CkOVM 1:4, insmc+tryp). The control dissolution profile in FIG. 18 and FIG. 19 represents insulin amount in the absence of trypsin. The area under the curve (AUC) for the dissolution profiles were calculated to compare the percentage of insulin available for absorption in the presence of trypsin in the case of solution and microsphere formulation. Comparison of the ratio of AUC values for insulin microcapsules at the end of 6 hours is shown in Table 17 (illustrating the cumulative amount of insulin available at the end of 6 hours in the presence of 0.5 μM trypsin and 0.1 μM chymotrypsin). From the table it can be seen that at the end of 6 hours, the percentage of insulin available for absorption from microcapsules is negligible. This may be explained by the kinetics of insulin degradation in the presence of trypsin.

From FIG. 18 it can be seen that the degradation of insulin solution is maximum in the first hour and slow thereafter. Insulin (%) remaining at the end of one hour is close to 45%. Assuming a linear degradation rate of insulin solution in the first hour, this corresponds to a rate of degradation of 0.46 IU/min. From FIG. 19, it can be seen that cumulative percentage insulin released from microcapsules in the first hour in the absence of trypsin is close to 60%. Assuming a linear rate of release in the first hour, this corresponds to a rate of release of 0.5 IU/min. Since the rate of degradation and rate of release are comparable, whatever insulin is released is degraded immediately. Due to this extensive degradation in the first hour itself, insulin concentration does not accumulate enough to be detectable even at the end of 6 hours.

In the presence of chicken ovomucoid, insulin (%) remaining for absorption increases in a concentration dependent fashion. From Table 17 it can be seen that insulin (%) available increases from 17.75% at 1:2 ratio of enzyme to inhibitor to 24.70% at 1:4 ratio. This represents a substantial improvement in the percentage of insulin available for absorption.

Stability of insulin in the presence of α-chymotrypsin is extremely poor. During the course of degradation of insulin solution in 6 hours, insulin remaining is almost reduced to a negligible value as shown in FIG. 18. Rate of degradation of insulin solution in the first hour is 0.75 IU/min while the rate of dissolution remains the same. Insulin solution available for absorption was calculated to be only 10.37% as shown in Table 17. Due to this reason, insulin released from microcapsules is extensively degraded and not detectable in the presence of α-chymotrypsin alone as shown by the flat line in FIG. 20 (illustrating the dissolution stability of insulin released from microcapsules in the presence of α-chymotrypsin and DkOVM). The reason may be the same as explained above.

In the presence of DkOVM, there is a considerable improvement in stability of insulin released from microcapsules at the end of 6 hours. Cumulative percentage of insulin remaining in the presence of various ratios of α-chymotrypsin and DkOVM is shown in FIG. 20. As shown in Table 17, the ratio of insulin remaining for absorption steadily increased from 23.3% in the presence of 1:1 concentration of enzyme to inhibitor to 42.3% at 1:4 ratio.

Oral delivery of proteins will be extremely difficult without the use of an absorption modifier. The absorption modifier may be an agent that increases permeability of the protein under study or an enzyme inhibitor that improves stability of the protein in the gastrointestinal tract. An enzyme inhibitor may be required to improve bioavailability of the protein even if permeation enhancement is the goal. There are many reports of bioavailability studies of insulin with enzyme inhibitors in various animal species such as rats (McPhillips et al. 1997), and dogs (Ziv et al. 1994). The amounts of enzyme inhibitors used have been selected without performing in vitro stability studies in the presence of pancreatic enzymes. Since variations exist in concentration of pancreatic enzymes with regards to the species under study, it may be helpful to evaluate the stability beforehand.

In vitro dissolution stability may serve as an effective screening tool to evaluate the effectiveness of various concentrations of inhibitor in the presence of enzymes. This will allow incorporation of practical amounts of enzyme inhibitor targeted towards inhibition of pancreatic enzymes. In vitro dissolution stability of a protein in the presence of enzymes has not been reported. Single time point measurements have been used in some studies to evaluate the in vitro efficacy of inhibitors against specific enzymes (et al. 1992 a; Bernkop-Schnurch et al. 1997). The parameter used in our study, percentage of protein available for absorption, may serve as a better indicator for evaluating the efficacy of inhibitors because it incorporates the complete dissolution profile in the calculation.

TABLE 17
Cumulative amount of insulin available at the end of 6 hours
in the presence of 0.5 μM trypsin and 0.1 μM chymotrypsin

| Formulation | AUC (% * hr) | Ratio (%) |
|---|---|---|
| Solution (sol) | | |
| Sol (no enzymes) | 600 | |
| Sol + Trypsin (T) | 261.33 | 43.55 |
| Sol + chymotrypsin (CT) | 62.24 | 10.37 |
| Microcapsules | | |
| Control (no enzymes) | 479.36 | |
| MC + T | Beyond detection limit | Beyond detection limt |
| MC + T + CkOVM (1:2) | 85.08 | 17.75 |
| MC + T + CkOVM (1:4) | 118.45 | 24.70 |
| Microcapsules | | |
| MC (no enzymes) | 479.36 | |
| MC + CT | Beyond detection limit | Beyond detection limit |
| MC + CT + | 111.71 | 23.3 |
| DkOVM (1:1) | | |
| MC + CT + | 190.81 | 39.40 |
| DkOVM (2:1) | | |
| MC + CT_DkOVM | 202.95 | 42.3 |
| (4:1) | | |

### EXAMPLE 4

### Characterization of the Microcapsules

Size Exclusion Chromatography

Insulin stock solution was prepared by dissolving insulin powder in 0.01 N HCl to obtain a stock solution of 100 IU/mL. Diluted solutions were made with pH 6.8 buffer between the concentration range 2-10 IU/mL. Physical mixture of insulin and Eudragit L100 was prepared by adding 47.5 mg Eudragit to 10 mL of 2 IU/ml stock solution of insulin. Microcapsule samples were prepared by dissolving 50 mg of optimized microspheres in 10 ml of pH 6.8 buffer. The chromatographic conditions are shown in Table 18 (size exclusion HPLC method for the analysis of insulin).

TABLE 18
Size Exclusion HPLC method for the analysis of insulin

| | |
|---|---|
| Column | Waters HMWP C₁₈ (7.8 × 300 mm) |
| Mobile phase | Mixture of Water 65%, Glacial Acetic Acid 15% |
| | v/v and Acetonitrile 20% v/v |
| | containing 0.1% L-Arginine (A) |
| Isocratic conditions | 100% A for 30 mins |
| Flow rate | 0.5 mL/min |
| Detection wavelength | 275 nm |
| AUFS | 0.001 |
| Injection volume | 100 μL |

Differential Scanning Calorimetry

Differential scanning calorimetry (DSC) was performed for insulin powder, physical mixture of insulin and Eudragit L100 and optimized microcapsules of insulin. The instrument used was DSC7. The instrument was calibrated using indium standards. 3 to 20 mg of samples were accurately weighed in small aluminum pans. The pans were covered with aluminum lids and then sealed. An empty aluminum pan similarly sealed was used as a reference. Samples were heated from 50° C. to 250° C. at a scan rate of 10° C. per minute in an atmosphere of nitrogen. After completion of the run, the thermograms were normalized to one milligram weight and their slopes optimized. The thermograms were plotted using a plotter. The melting endotherms of the peaks were recorded.

Fourier Transform Infrared Spectroscopy (FT-IR)

FT-IR spectroscopy was done with an Attenuated Total Reflectance (ATR) accessory. The samples analyzed were insulin powder, physical mixture of insulin and polymer and optimized microcapsules. The samples were run on a on-bounce diamond ATR accessory DurasampleIR. Insulin sample (4 mg/ml) was prepared by dissolving in a 50:50 mixture of 0.1 N HCl and alcohol. Polymer sample (4 mg/ml) was prepared by dissolving in alcohol. A 2% solution and a 50% solution of insulin in with polymer was prepared to represent the physical mixture. Microcapsules (5 mg equivalent to 69.86 μg/ml) were dissolved in a 50:50 mixture of alcohol and phosphate buffer pH 6.8. A drop of liquid was applied to the center of the crystal and allowed to dry for 2 min. The scanning range was 4000-400 cm⁻¹ at a resolution of 1 cm⁻¹. Spectra were represented as % transmittance on a common scale. Samples were run on a FT-IR model Impact 410.

Powder X-Ray Diffraction

Insulin (50 mg) was suspended in acetone and deposited on a glass slide. This was kept aside until the acetone evaporated. Samples of Eudragit L100, physical mixture of insulin and Eudragit L100, and microcapsules were run by placing the powder on the sample. Samples were obtained using a Philips Norelco Diffractometer fitted with a copper target. Measurements were carried out using 40 kV voltage and 20 mA current. Samples were scanned from 10°2θ to 40°2θ at a rate of 2θ/min. The scale factor used was 2000.

Results and Discussion of the Characterization of Microcapsules

Differential Scanning Calorimetry

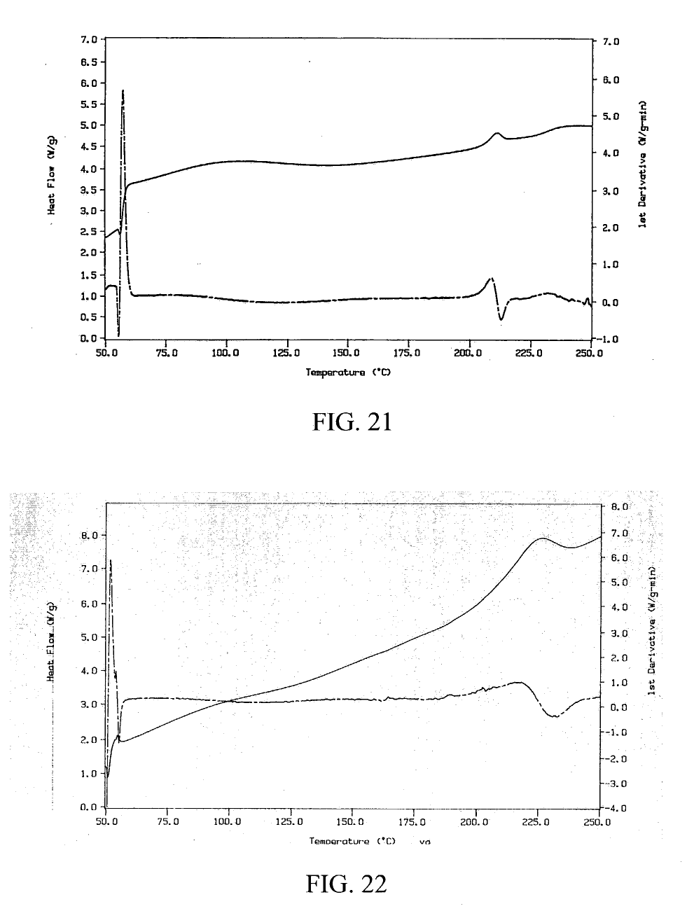

FIGS. 21, 22, 23 and 24 represent the DSC thermograms of insulin powder, polymer (Eudragit L100), physical mixture of insulin and polymer, and microcapsules of insulin. The solid lines represent the original thermograms and the dashed lines represent the processed thermograms (first derivative of the original thermogram).

From FIG. 21 (DSC thermogram of insulin powder, including the normal thermogram (solid line) and the first derivative processed thermogram (dashed line)), it can be seen that the insulin thermogram is characterized by broad and weak endotherms within the temperature range of 100-150° C. and a melting endotherm (Tₘ) in the range of 200-225° C. Both types of endotherms observed are irreversible. This means that if a sample is scanned through the endotherm and then cooled and re-scanned, the second scan does not show the endotherm. These kinds of broad endotherms have been observed for freeze dried human growth hormone, bovine somatotropin and several other proteins (Bell et al. 1995). All of these thermograms are characterized by the absence of a sharp increase in baseline near the glass transition temperature. Proteins melt in solid state at high temperature due to extensive degradation that occurs due to unfolding.

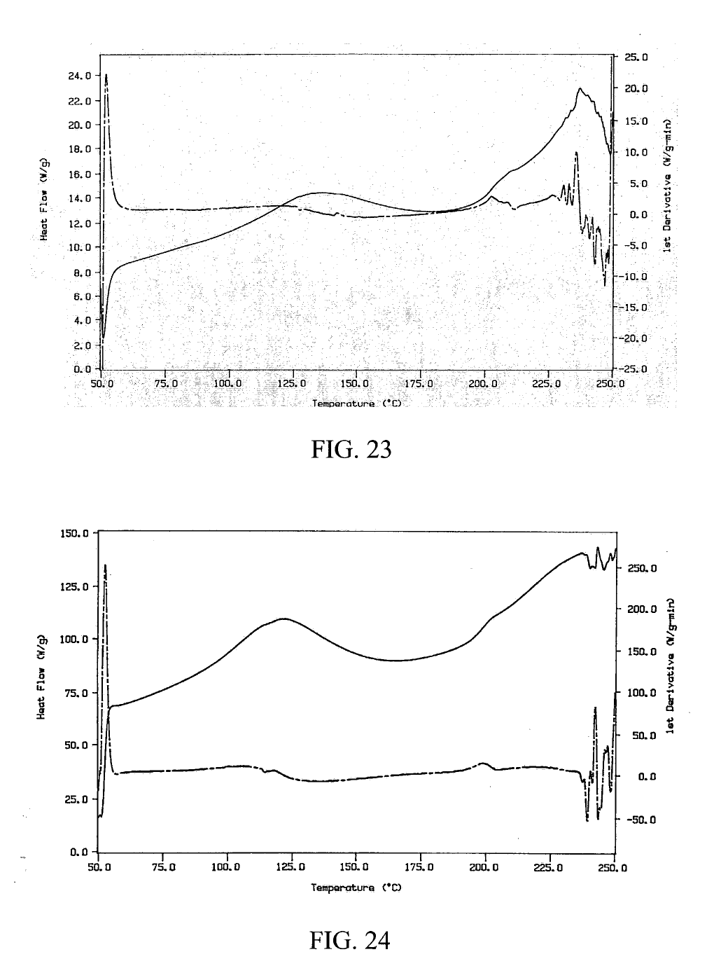

The advantage of processing of the endotherms by calculating the first derivative of the original thermogram include improved accuracy, improved peak resolution and quantitative determinations (Ford & Timmins 1989). Odd derivative curves (1^{st}, 3^{rd}, 5^{th} etc.) are especially useful in resolution enhancement of single and overlapping curves. This is especially useful to get the onset of melting temperature in the present case where the thermograms of insulin and polymer overlap in the 200-225° C. region and insulin loading in the physical mixture and microcapsules is extremely low (<2% of total weight). The calculation of onset of denaturation temperature is difficult from the original thermograms of physical mixtures (FIG. 22, illustrating the DSC thermogram of Eudragit L100, both for the normal thermogram (solid line) and the first derivative processed thermogram (dashed line)) and microcapsules of insulin (FIG. 23, illustrating the DSC thermogram of physical mixture of insulin and Eudragit L100, both the normal thermogram (solid line) and the first derivative processed thermogram (dashed line)). From the processed thermograms, the onset of melting temperature of insulin in pure form was calculated to be 209° C. (FIG. 21). The onset of melting shifted to 201° C. in the case of physical mixture and microcapsules of insulin (FIG. 22 and FIG. 23). This indicates that there may be physical interaction between insulin and polymer in the solid state. However, this interaction should be confirmed by other characterization procedures. FIG. 24 illustrates the DSC thermogram of microcapsules of insulin, both the normal thermogram (solid line) and the first derivative processed thermogram (dashed line).

Conditions that cause an increase in the Tₘ for a particular protein promote greater physical stability for the protein by providing greater resistance to thermal denaturation. Excipients that cause an increase in Tₘ provide for greater physical stability and excipients that cause a decrease in Tm have been found to decrease physical stability (Lee & Timasheff 1981; Lee & Lee 1987; Manning et al. 1989). The data from the DSC experiments should be interpreted with caution. It has been reported that some proteins undergo aggregation and precipitation upon thermal denaturation. Also, broadening of peaks leading to a shift in area, onset or peak temperature are simply due to mixing of components without indicating an interaction (Ford & Timmins 1989).

Powder X-Ray Diffraction

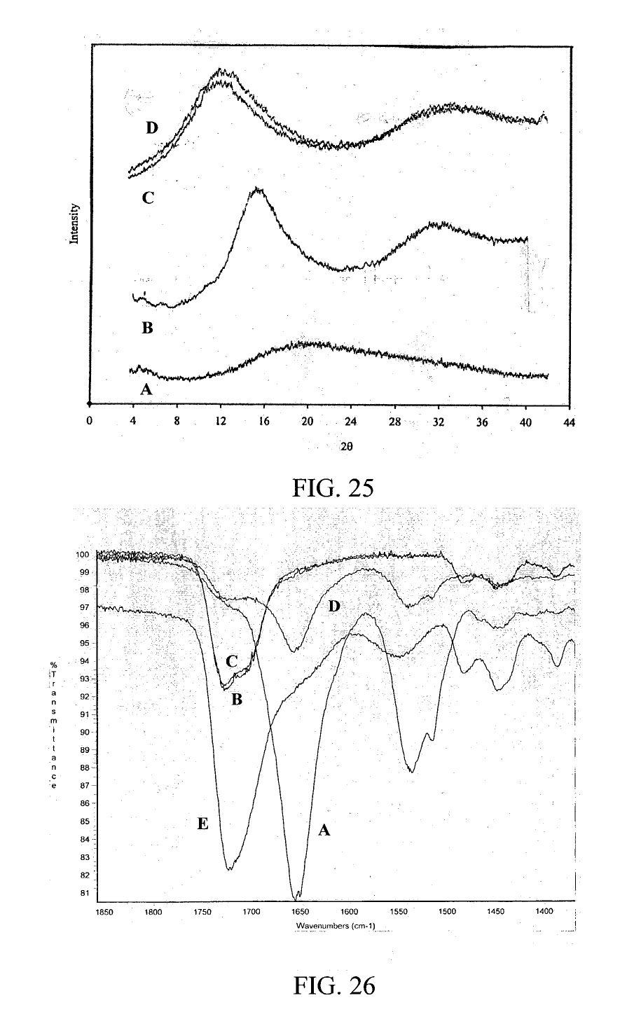

Powder x-ray diffractograms of insulin (A), polymer (Eudragit L 100) (B), physical mixture of insulin and polymer (C), and microcapsules of insulin (D) are shown in FIG. 25. The diffractogram of insulin (A) is associated with low intensity or broad peaks indicating lack of crystalinity. This is consistent with the observation that most of the proteins are isolated by lyophilization as amorphous powders. The diffractogram of polymer (B) is devoid of sharp peaks indicating its amorphous nature. The diffractograms of physical mixture (C) and microcapsules (D) are almost identical. It is difficult to ascertain if there is any change in the structure of the insulin in the microcapsules from the x-ray data.

Fourier Transform Infra Red Spectroscopy

FT-IR scans of insulin (A), polymer (B), physical mixture of 2% insulin and polymer (C), physical mixture of 50:50 insulin and polymer (D), and insulin microcapsules (E) are shown in FIG. 26. The band at 1659 cm⁻¹ in the case of insulin (A) corresponds to the alpha-helix region of the secondary structure. The spectra of polymer (B) shows a characteristic peak of the carboxyl group at 1705 cm⁻¹ and of the esterified carboxyl group at 1730 cm⁻¹. From the figure, it can be seen that the spectra of 2% mixture of insulin and polymer (C) and microcapsules of insulin (E) are identical. The band of alpha-helix is missing from both spectra. This result should be interpreted with caution. Attenuation of alpha-helix band is associated with a change in secondary structure of protein (Pikal & Rigsbee 1997). In the present case, it is possible that the insulin loading was below the detection limits of the instrument. To strengthen this argument, spectra of a 50:50 mixture of insulin and polymer (D) were generated. The appearance of the alpha-helix band exactly at 1659 cm⁻¹ is clearly seen in the figure. This indicates that the presence of polymer does not alter the secondary structure of insulin. The spectra of microcapsules is difficult to interpret due to the low loading of insulin. It does not clearly indicate the presence of insulin alpha-helix band.

Size Exclusion Chromatography

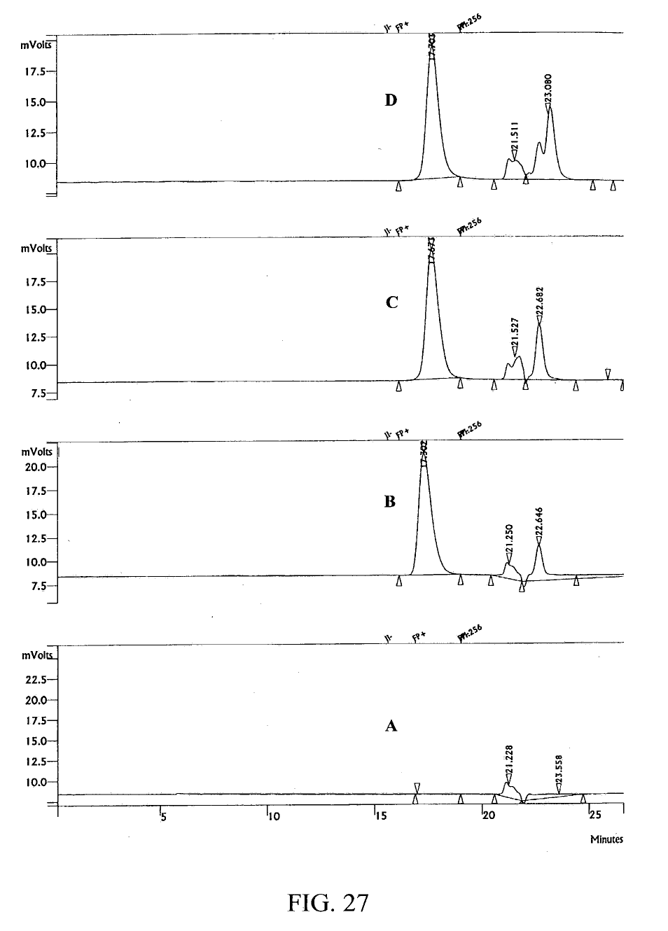

SEC chromatograms of blank (A), insulin (B), physical mixture of 2% insulin and Eudragit L100 polymer (C), and insulin extracted from microcapsules in phosphate buffer (pH 6.8) (D) is shown in FIG. 27. The insulin peak at 17.3 min corresponds to the monomeric form. Dimers and higher order aggregates are have not been observed at the concentration studied. The presence of polymer or processing conditions could have led to formation of covalent aggregates in the mixture or microcapsules. If they were formed, the dimers would have eluted before the peak of insulin. From the chromatograms of insulin in the physical mixtures and that extracted from the microcapsules, it can be seen that there are no additional peaks. This indicates that aggregate formation did not occur due to the presence of polymer or processing conditions.

### EXAMPLE 5

### Oral Dosage Form of Insulin and Duck Ovomucoid Optimization of Process Variables

HPLC Analysis of Insulin and Duck Ovomucoid

Insulin stock solution was prepared by dissolving insulin powder in 0.01 N HCl to obtain a final concentration of 100 IU/ml. Diluted concentrations were made in 1% v/v TFA/pH 6.8 buffer or pH 6.8 buffer alone using this stock solution within the range 0.05 IU/ml-1 IU/ml (1 IU=34.84 μg). Duck ovomucoid was dissolved in pH 6.8 phosphate buffer to make a stock of 1 mg/ml. Diluted stock solutions were made in the range 25-100 μg/ml. Chromatography conditions for simultaneous analysis of insulin and duck ovomucoid by a gradient HPLC method are given in Table 19.

TABLE 19
Chromatography conditions for simultaneous analysis of insulin
and duck ovomucoid by a gradient HPLC method

| | |
|---|---|
| Column | Vydac 218MS54 (C₁₈ (250 × 4.6 mm) 5μ 300 Å) |
| Mobile phase | (A) Water (0.05% v/v TFA), |
| | (B) Acetonitrile (0.05% v/v TFA) |
| Gradient conditions | 20% B for 5 min, 20-56% B in the next 15 min, |
| | 56% B-20% B in the next 10 min |
| Flow rate | 1 ml/min |
| Detection wavelength | 210 nm |
| AUFS | 0.001 |
| Injection volume | 100 μl |

Design of Experiment

A three factor three level Box Behnken design was created using the optimization software X-STAT 2.0. The independent and dependent variables studied in the Box Behnken design are given in Table 20. The design generated 15 experiments that included 3 replicates. All the experiments were run in duplicates to obtain the values of dependent variables. Mathematical relationships were generated to study the effect of independent variables on dependent variables studied.

Preparation of Tablet Containing Insulin and Duck Ovomucoid

Microcapsules of insulin with polymer were prepared according to the procedure described under the section entitled: Microencapsulation by coprecipitation described in Example 3 with some variations. During the preparation of microcapsules, the rate of addition of water and the volume of water with respect to the polymeric solution was varied according to the levels specified in Table 20. After the preparation of the microcapsules, they were mixed with lactose, talc and magnesium stearate and compressed at the compression pressures listed in Table 20. The composition of the tablet was 125 mg of microcapsules equivalent to 50 IU of insulin, 175 mg lactose, 6 mg of talc, 3 mg of magnesium stearate and 10 mg of duck ovomucoid. These ingredients were mixed geometrically and then compressed in a microprocessor controlled single station carver press using a die and punch set contained in a customized holder. The dwell time was constant at 2 sec. The punch used was flat-faced with a diameter of ¾″. The tablets were immediately used for dissolution studies.

Drug Encapsulation Efficiency

The protocol used for drug encapsulation is previously described in Example 3. Experiments were performed in triplicate.

Dissolution Studies

The protocol used for dissolution studies is described in Example 3 with minor modifications. Dissolution experiments were done in duplicate for each run in the experimental design. The release of insulin and DkOVM was monitored for the duration of the dissolution studies.

TABLE 20
Variables in the Box Behnken Design

| | Low (−1) | High (1) |
|---|---|---|
| Independent Variables | | |
| X₁ = rate of addition of polymer (mL/min) | 10 | 20 |
| X₂ = compression Pressure (tons) | 0.6 | 1.2 |
| X₃ = volume of Water (ml) | 50 | 150 |
| Dependent Variables | | |
| Y₁ = release (%) after 1 hour | | |
| Y₂ = release (%) after 2 hour | | |
| Y₃ = release (%) after 3 hour | | |
| Y₄ = release (%) after 4 hour | | |
| Y₅ = release (%) after 5 hour | | |
| Y₆ = release (%) after 6 hour | | |
| Y₇ = drug encapsulation efficiency | | |

Results and Discussion of the Optimization of an Oral Dual Controlled Release Tablet Dosage Form of Insulin and Duck Ovomucoid for Protection Against Enzymatic Degradation

HPLC Analysis of Insulin and Duck Ovomucoid

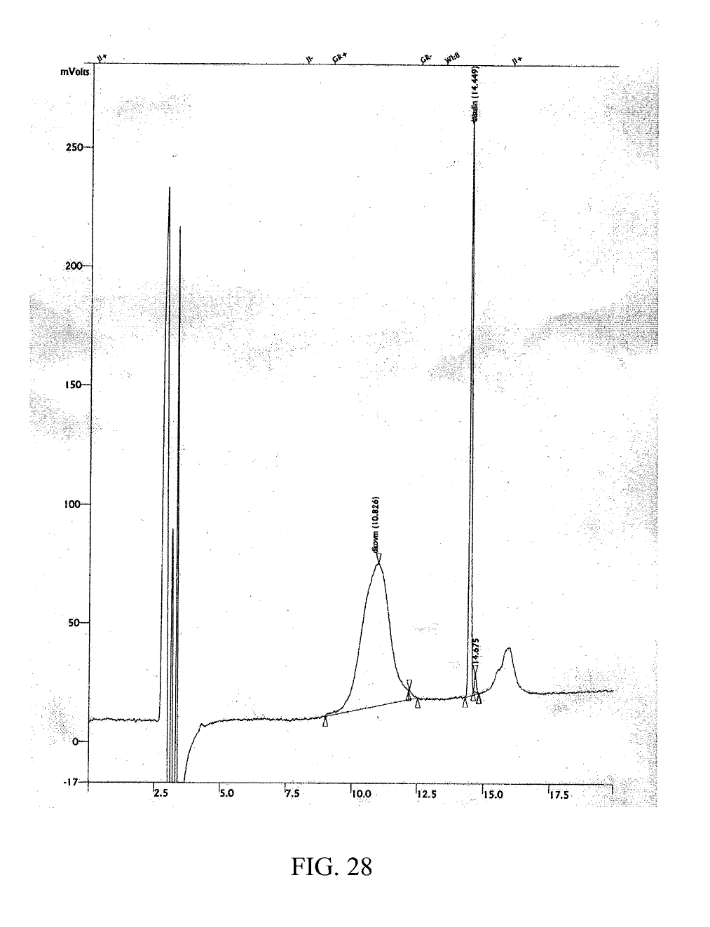

A representative dissolution profile chromatogram of the separation of insulin and duck ovomucoid is shown in FIG. 28. The peak eluting at 10.826 min corresponds to DkOVM and that eluting at 14.449 min corresponds to insulin. From the chromatogram it can be seen that they are well separated. The range of standard curve for the analysis of insulin was between 0.05 IU/ml-1.0 IU/ml. For this range the slope value of a typical run was 3150330.5 and the intercept value was −37870.6. The correlation coefficient (r²) was 0.998. The range of standard curve for the analysis of DkOVM was 25-100 μg/ml. For this range, the slope value of a typical run was 62161.28 and the intercept value was −10887.2. The correlation coefficient (r²) was 0.997.

Optimization of Process Variables for Insulin

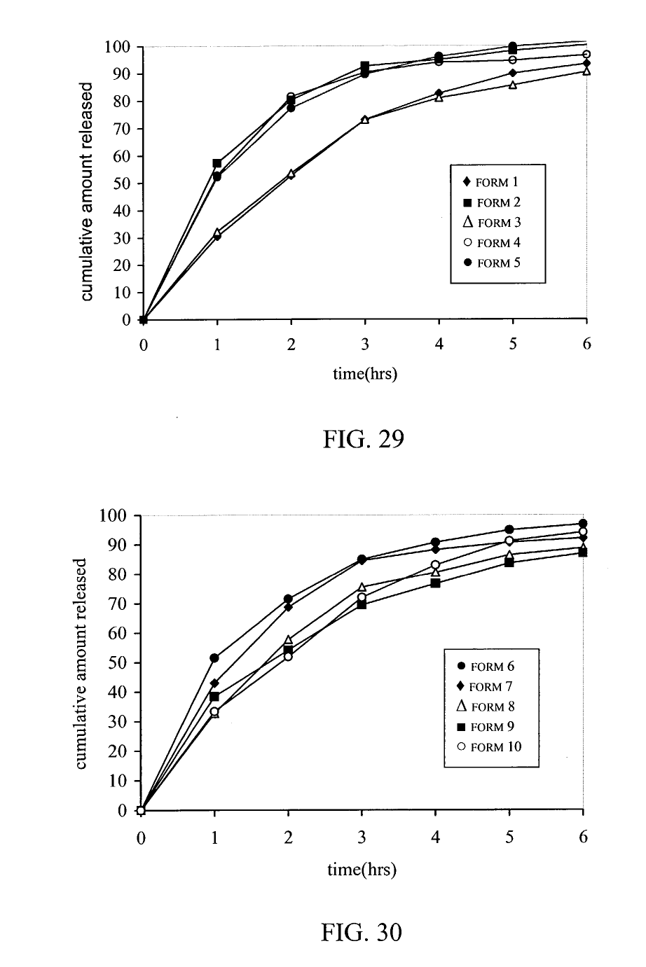

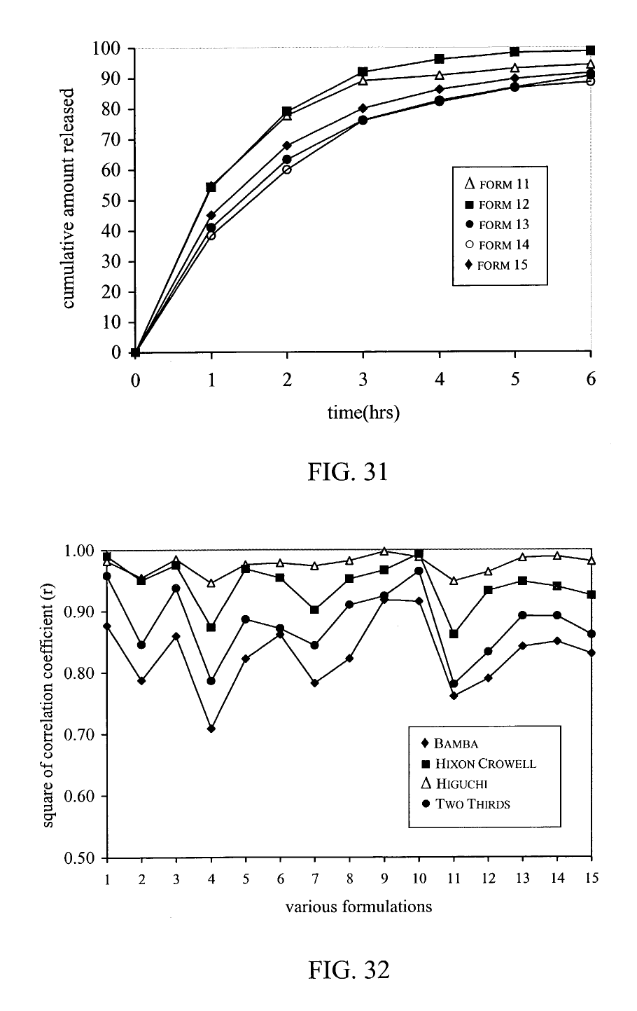

The experimental runs and the observed responses for the Box Behnken design for the 15 formulations are shown in Table 21. The dependent variables studied were cumulative amount released starting from 1 hr (Y₁) up to 6 hours (Y₆) and drug encapsulation efficiency (Y₇). The experimental design generated various factor combinations that resulted in different release rates of insulin and encapsulation efficiencies. From the Table 21 it can be seen that Y₁ varies from a minimum value of 30.44 in experiment #1 to a maximum value of 57.28 in experiment #2. FIG. 29 (dissolution profiles of insulin from formulations 1-5 of the experimental design), FIG. 30 (dissolution profiles of insulin from formulations 6-10 of the experimental design), and FIG. 31 (dissolution profiles of insulin from formulations 11-15 of the experimental design) represent the dissolution profiles of the experimental formulations.

TABLE 21
Experimental runs and observed responses for the Box Behnken Design

| Form No. | X₁ | X₂ | X₃ | Y₁ | Y₂ | Y₃ | Y₄ | Y₅ | Y₆ | Y₇ |
|---|---|---|---|---|---|---|---|---|---|---|
| 1 | 20 | 1.2 | 100 | 30.44 | 52.73 | 73.16 | 82.62 | 89.9 | 93.38 | 94.52 |
| 2 | 20 | 0.6 | 100 | 57.28 | 80.26 | 92.69 | 94.97 | 98.21 | 100.23 | 94.52 |
| 3 | 10 | 1.2 | 100 | 32.24 | 53.62 | 73.09 | 80.97 | 85.58 | 90.47 | 96.32 |
| 4 | 10 | 0.6 | 100 | 52.8 | 81.55 | 90.41 | 94.03 | 94.59 | 96.56 | 96.32 |
| 5 | 20 | 0.9 | 150 | 52.2 | 77.3 | 89.66 | 96.12 | 99.7 | 101.53 | 91.83 |
| 6 | 20 | 0.9 | 50 | 51.48 | 71.44 | 84.93 | 90.66 | 94.83 | 96.78 | 95.58 |
| 7 | 10 | 0.9 | 150 | 42.95 | 68.68 | 84.49 | 88.17 | 90.68 | 92.09 | 96.36 |
| 8 | 10 | 0.9 | 50 | 32.74 | 57.7 | 75.43 | 80.42 | 86.38 | 88.79 | 93.41 |
| 9 | 15 | 1.2 | 150 | 38.44 | 54.11 | 69.37 | 76.57 | 83.64 | 86.94 | 100.5 |
| 10 | 15 | 1.2 | 50 | 33.33 | 51.83 | 71.94 | 82.97 | 91.14 | 94.1 | 91.83 |
| 11 | 15 | 0.6 | 150 | 54.81 | 77.69 | 89.03 | 90.84 | 93.12 | 94.32 | 96.36 |
| 12 | 15 | 0.6 | 50 | 54.34 | 78.98 | 91.82 | 96.1 | 98.25 | 98.68 | 91.83 |
| 13 | 15 | 0.9 | 100 | 41.08 | 63.3 | 76.13 | 82.9 | 86.88 | 90.64 | 105 |
| 14 | 15 | 0.9 | 100 | 38.51 | 60 | 76.04 | 82.16 | 86.71 | 88.58 | 105.4 |
| 15 | 15 | 0.9 | 100 | 45.16 | 67.88 | 80.02 | 86.24 | 89.72 | 91.61 | 103.6 |

Kinetics of dissolution of coprecipitates has been estimated by different models depending upon the polymer studied. The model for diffusion controlled release is given by Higuchi (Higuchi 1963) is M=k*sq.root t, where M is the percentage of drug dissolved and k is the dissolution rate constant, and t is the time for dissolution. If the drug is released by a dissolving gel-like layer formed around the drug during the dissolution process, the equation LnM=kt proposed by Bamba et al. is used (Bamba et al. 1979). The equation proposed by Hixon Crowell (Hixon & Crowell 1931) Mo^{1/3}−M^{1/3}=kt for the dissolution of powders assumes that dissolution of the powder is independent of the intial particle diameter (M in this equation represents the amount of drug left undissolved). The “two-third” model or the modified cube-root equation (Niebergall & Goyan 2000) represented by M₀^{2/3}−M^{2/3}=kt takes into account the changing surface area of the granulated material during dissolution.

To obtain the kinetics of drug release, the data from all of the 15 experiments were fitted to the 4 equations discussed above. FIG. 32 shows the plot of square of correlation coefficient versus time of all of the 15 formulations (FIG. 32 illustrates the fitting of dissolution kinetic models to the experimental formulations: Higuchi's square root of time, Hixon Crowell, Two Thirds, and Bamba). From FIG. 32 it can be seen that the square of correlation coefficient is closest to the Higuchi's square root of time model. This model was selected as representative of the dissolution kinetics of all of the formulations. Similar kinetics of dissolution profile was observed in the case of ibuprofen and indomethacin coprecipitates that were compressed into tablet matrices (Kamachi et al. 1995b).

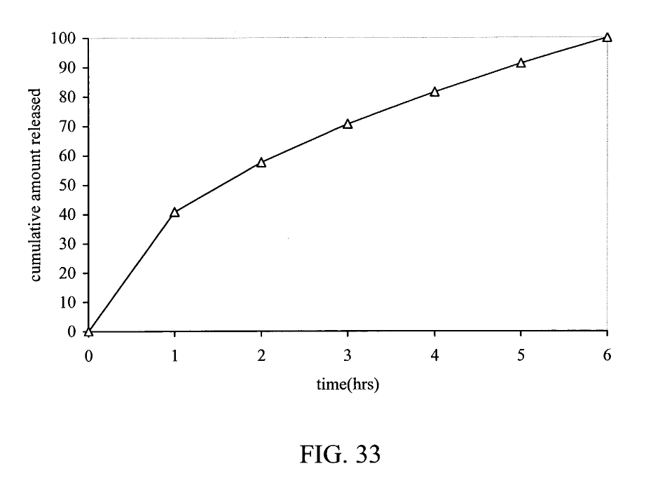

The dependent variable selected for optimization was Y₆ (cumulative amount of drug released at the end of 6 hours). A theoretical profile of Y₆ was generated from the Higuchi's square root of time model with an aim of 100% release at the end of 6 hours (FIG. 33, theoretical profile of dissolution of insulin after fitting to the dissolution kinetics model). Based on the theoretical values of Y₁-Y₆ obtained from this profile the following constraints were put on each dependent variable studied: 35.82<Y₁<45.82; 52.73<Y₂<62.73; 65.71<Y₃<75.71; 76.65<Y₄<86.65; 86.28<Y₅<96.28; Y₆>90; Y₇>95. These values were used as input to define the upper and lower limits on the dependent variables to fit the data to the model constructed. Within these constrained optimization conditions, the statistical package generated the mathematical relationships for Y₁-Y₇. A representative equation is Y₆=90.28+2.99X₁−3.11X₂−0.44X₃−0.18 X₁X₂+0.36 X₁X₃+0.70 X₂X₃+3.08X₁²+1.80X₂²+1.44X₃².

The above equation represents the quantitative effect of the factors studied on the response Y₆. The values of the variables X₁-X₃ relate to the effect of the factors on the response. Interaction terms are represented by coefficients with more than one factor term and quadratic nature of relationship is represented by second order terms. The positive and negative signs of the various terms represent a synergistic and antagonistic effect of factors on the response.

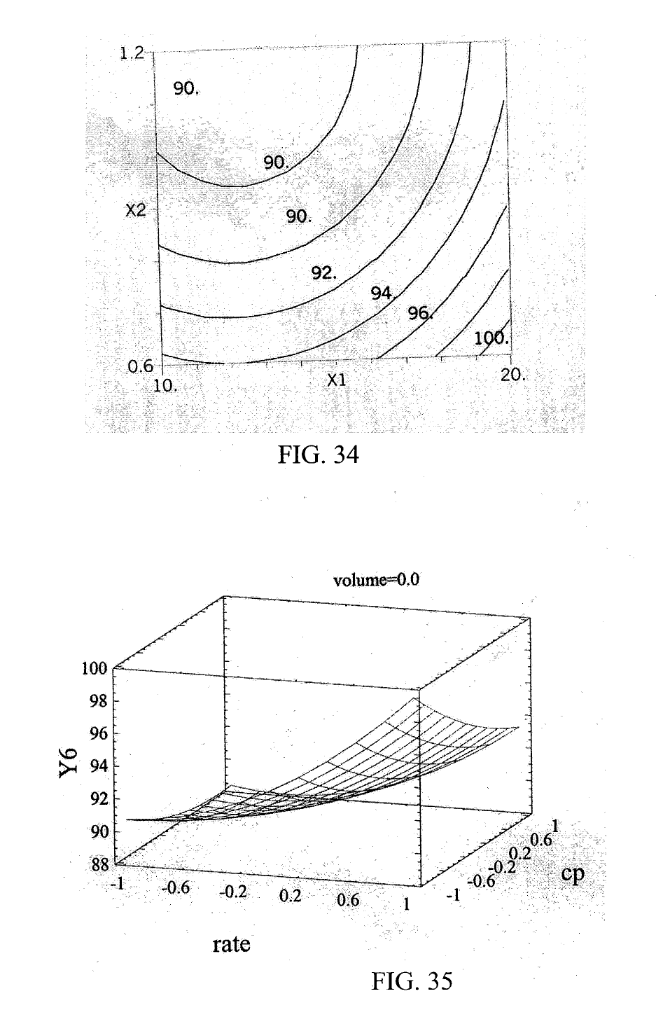

The relationship between factors and responses can be further understood by contour and response surface plots. FIG. 34 is a contour plot that shows the effect of X₁ (rate of addition) and X₂ (compression pressure) on Y₆ (cumulative amount of drug released at the end of 6 hours). The lines in the contour plot are curvilinear indicating the possibility of an interaction between X₁ and X₂. From the figure it can be seen that as the rate of addition is increased, the cumulative amount of insulin released is higher at low compression pressures when compared to high compression pressure.

FIG. 35 is a response surface plot to explain the interaction effect of rate of addition, X₁ (normalized), and compression pressure, X₂, (normalized) on Y₆, cumulative amount of drug released at the end of 6 hours. As the rate of addition is increased from 10 ml/min to 20 ml/mm, Y₆ increases from 88% to 94% at low levels of X₂. On the other hand, Y₆ increases from 88% to 94% at higher levels of X₂. The effect of rate of addition and compression pressure is opposite to each other as seen in polynomial equation. It appears that microcapsules obtained at high rate of addition are not susceptible to delay in release by compression pressure at low levels. This will also explain the decrease in release at high rate of addition and higher compression pressure.

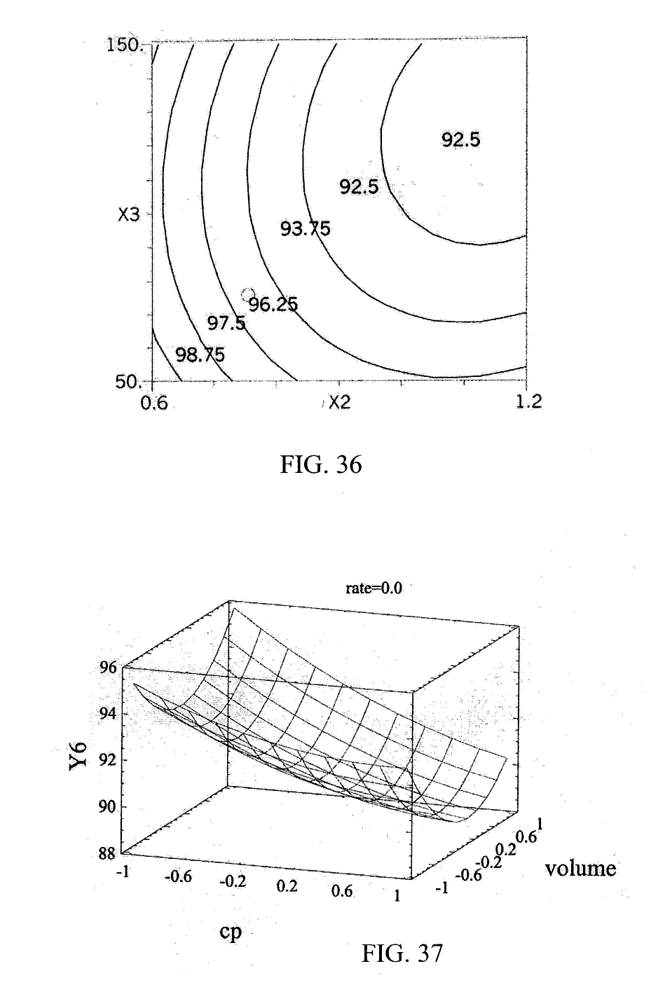

The contour plot in FIG. 36 explains the effect of X₂ (compression pressure) and X₃ (volume of water with respect to polymeric solution) on Y₆ (cumulative amount of drug released at the end of 6 hours). At low compression pressure and low volume of water the cumulative amount of insulin released is higher. The cumulative amount of insulin released at high compression pressure and high volume of water is less. Since the goal of the optimization experiment is to have maximum amount of drug release, the region of low compression pressure and low volume of water is favorable.

This relationship can be further explained by the response surface plot in FIG. 37 (showing the effect of compression pressure (X₂) (normalized) and volume of water with respect to polymeric solution (X₃) (normalized) on cumulative amount of drug released at the end of 6 hours (Y₆)). At low level of volume of water, as the compression pressure is increased from 0.6 tons to 1.2 tons, the response decreases from 95% to 92%. At high level of volume of water the response decreases from 96% to 90% when the compression pressure is increased from low to high.

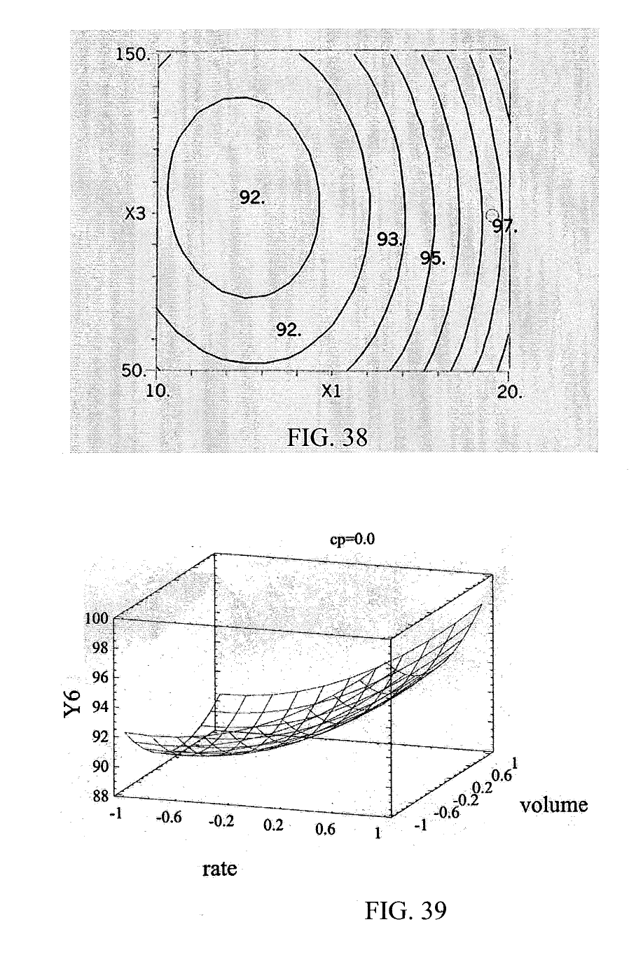

FIG. 38 (showing effect of rate of addition (X₁) and volume of water with respect to polymeric solution (X₃) on cumulative amount of drug released at the end of 6 hours (Y₆)) is a representative contour plot that shows the effect of X₁ and X₃ on Y₆. At low rate of addition and high volume of water the encapsulation efficiency is better. This leads to lower dissolution values of Y₆ probably due to the uniformity of polymer around the drug. The goal of the optimization process was to maximize Y₆. This is possible at higher flow rate and higher volume of water.

This can be further explained by the response surface plot in FIG. 39 (showing the effect of volume of water with respect to polymeric solution (X₃) (normalized) and rate of addition (X₁) (normalized) on cumulative amount of drug released at the end of 6 hours (Y₆)). At low level of volume of addition, as the rate is increased from 10 ml/min to 20 ml/min, the cumulative amount of drug released increases from 92.5% to 96%. At high level of volume of addition, the cumulative amount of insulin released increases from 90.5% to 98%. The rate of increase is more at high level of addition when compared to low level of addition.

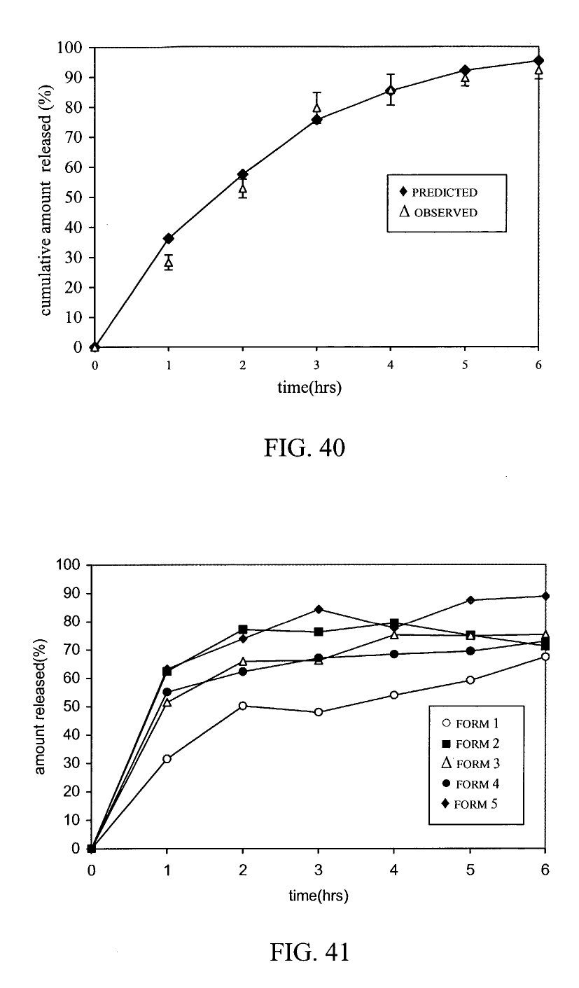

To achieve the goal of maximizing drug release at the end of 6 hours, a combination of factors was selected by an optimization process. The software generated values of X₁=20 ml/min, X₂=1.2 tons and X₃=80.7 ml to achieve a theoretical profle as shown in FIG. 40 (comparison of observed and predicted dissolution profiles of the optimized formulation of insulin). Two batches were run with the above parameters and the dissolution profiles were generated. The observed dissolution profile was compared to the theoretical dissolution profile (FIG. 40). From the figure it can be seen that they are in close agreement.

Dissolution Studies of DkOVM

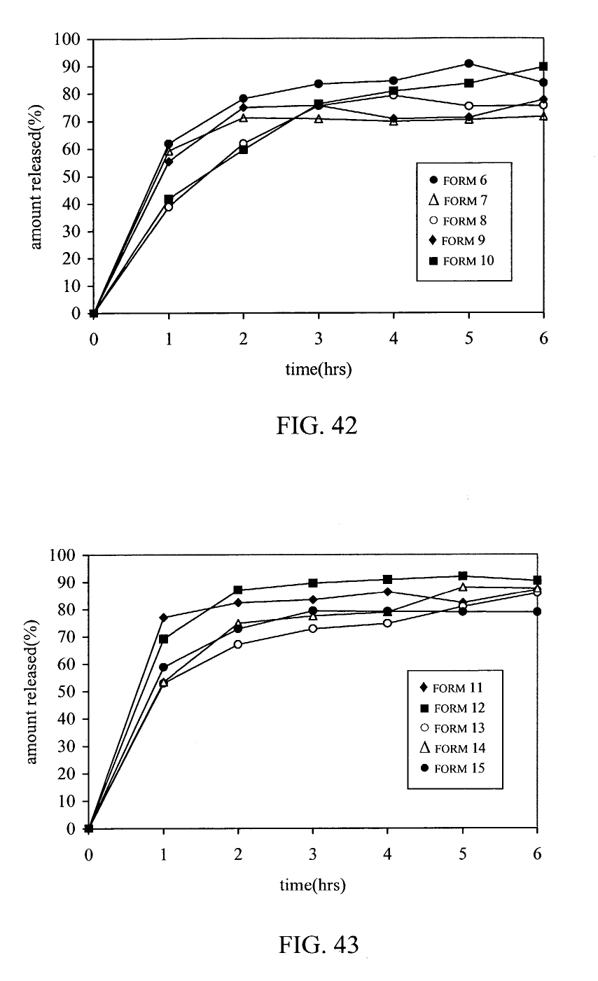

DkOVM dissolution was also followed for all the formulations studied. Table 22 represents the cumulative amount of DkOVM released at various time points for all the experimental formulations. DkOVM is present in the form of a powder that is compressed within a polymeric system. Representative dissolution profiles of DkOVM dissolution from the tablet matrix for all the formulations are shown in FIG. 41 (dissolution profiles of DkOVM from formulations 1-5 of the experimental design), FIG. 42 (dissolution profiles of DkOVM from formulations 6-10 of the experimental design), and FIG. 43 (dissolution profiles of DkOVM from formulations 11-15 of the experimental design).

The formulation of insulin obtained in the present study is intended for administration by the oral route. To counter the action of enzymes, DkOVM has been incorporated as an enzyme inhibitor. Among the luminal enzymes in the gastrointestinal tract, insulin is extensively degraded by trypsin and α-chymotrypsin in the gastrointestinal tract. DkOVM stabilizes insulin against degradation by both the enzymes as discussed under enzymatic stability of insulin in the presence of trypsin and α-chymotrypsin. Capsule dosage forms have been tried with insulin and enzyme inhibitors for oral delivery (Morishita et al. 1992; Trenktrog et al. 1995). The enzyme inhibitor used in these studies was exposed spontaneously to the gastrointestinal enzymes whereas the release of insulin was sustained.

The present dosage form has the dual advantage of predictable release of insulin and delayed release of enzyme inhibitor. The delayed release of inhibitor might be particularly suitable for releasing the inhibitor where insulin is subjected to maximum degradation. This may offer better protection for insulin against digestive enzymes when compared to the capsule dosage forms. The advantages may include less spreading of inhibitor in the gastrointestinal tract due to dilution and extended inhibitory effect. This is supported by reports on permeation enhancers that performed better when introduced directly into the intestinal tract when compared to oral administration (Sinko et al. 1999; Ziv et al. 1994). Further, a gradual release of inhibitor may be the solution to toxicity problems due to high systemic levels of inhibitor.

It will be apparent to one skilled in the art that specific formulations obtained by methods utilizing coating of beads and direct compression may also provide dual controlled release.

TABLE 22
Cumulative amount of DkOVM released at various time points

| Form No. | % 1 hr | % 2 hr | % 3 hr | % 4 hr | % 5 hr | % 6 hr |
|---|---|---|---|---|---|---|
| 1 | 31.51 | 50.20 | 47.94 | 53.90 | 59.14 | 67.44 |
| 2 | 62.38 | 77.15 | 76.19 | 79.47 | 75.05 | 71.22 |
| 3 | 51.44 | 65.93 | 66.20 | 75.21 | 74.90 | 75.28 |
| 4 | 55.10 | 62.30 | 67.04 | 68.36 | 69.42 | 72.94 |
| 5 | 62.23 | 73.87 | 84.29 | 77.64 | 87.46 | 88.92 |
| 6 | 61.91 | 78.19 | 83.48 | 84.60 | 90.65 | 83.43 |
| 7 | 59.31 | 71.23 | 70.84 | 69.91 | 70.47 | 71.62 |
| 8 | 38.92 | 61.87 | 75.52 | 79.33 | 75.35 | 75.64 |
| 9 | 55.40 | 74.98 | 75.71 | 70.86 | 71.34 | 77.74 |
| 10 | 41.79 | 59.60 | 76.22 | 80.88 | 83.56 | 83.69 |
| 11 | 76.95 | 82.33 | 83.40 | 86.26 | 82.26 | 87.13 |
| 12 | 69.17 | 86.96 | 89.54 | 90.83 | 92.00 | 90.39 |
| 13 | 52.94 | 67.05 | 72.75 | 74.67 | 80.87 | 85.85 |
| 14 | 53.38 | 74.78 | 77.45 | 78.52 | 88.01 | 87.50 |
| 15 | 58.82 | 72.76 | 74.31 | 79.06 | 78.89 | 78.81 |

### EXAMPLE 6

### In Vitro Evaluation of Ovomucoids Protecting Salmon Calcitonin Against Metabolism by Serine Proteases

Introduction

With the advent of biotechnology, particularly the advances in recombinant protein technology, peptides and proteins have received much attention for their therapeutic roles. The majority of these drugs are commonly administered by the parenteral routes, which are often complex, difficult and painful. Hence, the oral administration of peptide and protein drugs would lead to a higher patient compliance being favored by patients, practitioners and pharmaceutical industry for reasons of ease and economics. (Shah R B et al. 2002).

Among these peptide drugs one of the most frequently used is calcitonin (CT), which is a polypeptide hormone with 32 amino acids. It is secreted by parafollicular cells (c-cells of the thyroid gland. It plays a crucial role in both calcium homeostasis and bone remodeling and enjoys popularity in the management of osteoporosis and Paget's disease. It causes hypocalcaemia by inhibiting the release of calcium from bone and by stimulating urinary calcium excretion. Four forms of CT are used clinically, namely, synthetic human CT (hCT), synthetic salmon CT (sCT), natural porcine CT (pCT), and a synthetic analogue of eel CT (eCT). Pharmacokinetics of these various forms of CT was recently reviewed. It delineates some facts about their fate and metabolism. The unique structure of sCT protects it against sequestration in the liver, muscle and bone. Even though sCT has been found to be resistant to breakdown by liver homogenates, the liver plays a significant role in the metabolism of pCT. As the evidence suggests, the hepatic metabolism of sCT is minimal and the rate-limiting step to successful oral administration of sCT is its delivery into the portal vein.

So far, CT has not reached its full market potential due to the inconvenience and pain associated with the injectable dosage forms and the low patient acceptance of the nasal delivery system. The oral route is a preferred route of administration considering the chronic nature of CT therapy. However, the extensive proteolytic degradation in the GI lumen and low intrinsic intestinal membrane permeability necessitates the use of high doses [4000-6000 IU/mg] of sCT, even though sCT is 30 times more potent than hCT [150-200 IU/mg]. However, apart from poor absorption from the gastrointestinal-tract, the oral bioavailability of calcitonin is strongly limited by the enzymatic degradation based on luminally secreted serine proteases.

Several approaches have been reported for enhanced permeation of sCT through biological membranes. One approach to overcome this so-called enzymatic barrier is the co-administration of protease inhibitors. It has been demonstrated that these auxiliary agents are very efficient in improving the oral bioavailability of peptides (Yamamoto et al., 1990). As described above, ovomucoids are enzyme inhibitors derived from the egg white of avian species. Extensive reviews of their source, active domains, and mechanism of inhibitory action are found elsewhere (Laskowski and Kato, 1960. Rhodes et al., 1960).

Avian ovomucoids are present in the egg whites of all birds and account for approximately 10% of egg white proteins. sCT is known to be rapidly degraded by trypsin and α-chymotrypsin (Dohi, M., et al., 1993; Lang et al. 1996, pp. 1679-1685) and elastase (Guggi and Bernkop-Schnurch, 2003), therefore, an inhibitor providing a strong protective effect towards these pancreatic serine-proteases is necessary. As the ovomucoids have been shown to inhibit these enzymes, they might have an extremely useful role in the oral delivery of sCT.

The aim of this study was, therefore, evaluation of ovomucoids efficacy for protecting sCT against metabolism by serine proteases, namely trypsin and α-chymotrypsin. The ovomucoids evaluated were chicken ovomucoid (CkOVM), duck ovomucoid (DkOVM) and turkey ovomucoid (TkOVM). A well-known protease inhibitor, aprotinin was used for comparison. The major metabolites of sCT by trypsin and α-chymotrypsin mediated degradation were characterized. The stability of sCT in homogenates of Caco-2 cells and goat small intestine were also evaluated to obtain preliminary information for in vitro permeability studies of proteins.

Methods

Stability studies of sCT in the Presence of Protease Inhibitors

sCT solutions (50 μM) were incubated at 37° C. in 50 mM Tris buffer containing 0.1 mM calcium chloride (pH 8.0). Degradation profiles generated in the presence of 0.5 μM trypsin and 0.1 μM α-chymotrypsin served as controls. The concentrations were selected on the basis of a reported study (Schilling R. J. and Mitra A. K. 1991). DkOVM and TkOVM were evaluated at different enzyme-to-inhibitor ratios to evaluate their inhibition of sCT degradation. CkOVM and aprotinin were used at 1:1 enzyme-to-inhibitor ratio in order to compare the efficacy of various protease inhibitors used. sCT and enzyme solutions were incubated for 15 min at 37° C. before starting the experiments. Samples were taken after 0, 5, 15, 30 and 60 min and immediately diluted with cold 1% trifluoroacetic acid (TFA) to reduce the pH to 2.5. The samples were analysed at 4° C. by HPLC given below. Plots of sCT remaining against time were generated. 160 values (amount of sCT (%) remaining after 60 min) were used to compare the efficacy of the ovomucoids. Similar studies were performed with aprotinin. The study was also conducted by incubating 50 μM sCT solutions with 0.5 μM trypsin and 0.1 μM α-chymotrypsin added together and the efficacy of TkOVM was evaluated at various ratios.

Stability Studies of sCT in the Presence of Caco-2 Cell and Goat Intestinal Homogenates

Preparation of Caco-2 Cell Homogenate

Human colon adenocarcinoma (Caco-2) cells were cultured, in an atmosphere of 5% CO₂ at 37° C. in T-75 tissue culture flasks using DMEM supplemented with 10% FBS, 100 U/ml penicillin, 100 μg/ml streptomycin, 1.25 ml human Transferrin. The medium was changed every other day until the flasks reached 90% confluence (3-4 days). Caco-2 cells of less than 20 passages were used. Caco-2 homogenate was prepared according to the method of Augustijns et al. with slight modification. Briefly, the Caco-2 monolayer was washed three times with ice-cold PBS (pH 7.4). The cells were scraped off with a tissue culture scraper on ice and homogenized in 1 ml ice-cold PBS using a mechanical homogenizer. The mixture was centrifuged at 12,000 rpm for 10 min at 4° C. The resultant supernatant was used as Caco-2 homogenate. The protein content was determined using BCA assay. Tryspin and chymotrypsin assay was done for the homogenate as described below.

Preparation of Goat Intestinal Homogenate

Goat intestinal homogenate was prepared using a reported method for preparation of intestinal homogenate with minor modifications (Yamamoto et al., 1990). Briefly, fresh goat intestinal tissues, obtained from a local slaughter house, were washed with phosphate buffered saline (PBS) pH 7.4 at 4° C. and stored at −80° C. Immediately before each experiment, specimens were thawed at room temperature and then were washed again with PBS at 4° C. 25 g of tissue was cut from the small intestinal region and was homogenized in 10 ml PBS at 4° C. by use of a mechanical homogenizer for 5 min. The homogenate was centrifuged at 5000 g in a refrigerated (4° C.) centrifuge for 10 min to remove cellular and nuclear debris. The resulting supernatant was assayed for protein content by BCA assay with bovine serum albumin as the standard. The supernatant was also assayed for trypsin and chymotrypsin content.

Trypsin Assay

Trypsin was assayed using BAEE as a substrate (Guggi and Bernkop-Schnurch, 2003) with some modifications. Briefly, trypsin was dissolved in PBS to a final concentration of 0.5 μM and the solution was incubated at room temperature. After addition of 0.34 mg of BAEE dissolved in 100 μl of PBS to 20 μl of trypsin solution (0.5 μM), the increase in absorbance caused by the hydrolysis of the substrate to N-α-benzoylarginine (BA) was recorded in a spectrophotometer at λₘₐₓ of 253 nm at 1 min intervals for 5 min. Thereafter, inhibitory efficacy of the TkOVM (4.8 μM) was performed similarly after addition of TkOVM to trypsin first before addition of BAEE.

Trypsin content was also assayed similarly in Caco-2 and goat intestinal homogenates.

α-Chymotrypsin Assay

α-Chymotrypsin was assayed using BTEE as a substrate (Guggi and Bernkop-Schnurch, 2003) with some modifications. Briefly, α-chymotrypsin was dissolved in PBS to a final concentration of 0.1 μM and the solution was incubated at room temperature. After addition of 0.25 ml of substrate solution (18.5 mg of BTEE dissolved in 31.7 ml of methanol and 18.3 ml of demineralised water) to 20 μl of α-chymotrypsin solution (0.1 μM), the increase in absorbance caused by the hydrolysis of the substrate to N-α-benzoyltyrosine (BT) was recorded in a spectrophotometer at λₘₐₓ of 254 nm at 1 min intervals for 5 min. Thereafter, inhibitory efficacy of the TkOVM (4.8 μM) was performed similarly after addition of TkOVM to α-chymotrypsin first before addition of BAEE. α-chymotrypsin content was also assayed in Caco-2 and goat intestinal homogenate by the same method.

Stability Studies in Homogenates

The degradation of sCT was studied by incubating the sCT solution to a final concentration of 50 μM with 900 μl Caco-2 cell and goat intestinal homogenates. The samples were withdrawn at 0 and 60 min and were diluted immediately with cold TFA 1%. The samples containing intestinal homogenate were centrifuged at 5000 g for 10 min to remove precipitated proteins. The supernatant was injected into HPLC to determine the sCT content.

Evaluation of Trypsin and Chymotrypsin Mediated Metabolites of sCT

Evaluation by HPLC:

The metabolites generated by trypsin and α-chymotrypsin were determined by RPHPLC as described below. Retention times of trypsin and α-chymotrypsin mediated sCT metabolites were 12.6 and 13.3 min, respectively.

Evaluation by Gel Electrophoresis

In order to determine the R_{f} values and the molecular weights of the metabolites, gel electrophoresis was performed. Solutions in Tris-HCl buffer (pH 8.0) of 0.5 μM trypsin, 0.1 μM α-chymotrypsin, a mixture containing 0.5 μM trypsin as well as 0.1 μM α-chymotrypsin and a mixture containing 0.5 μM trypsin, 0.1 μM α-chymotrypsin as well as 4.8 μM TkOVM were incubated at 37° C. with 150 μM sCT for 0.5 min, 15 min and 0.5 min, respectively. After the above specified times, the solutions were immediately diluted with cold 1% TFA and 8 μl of these samples were diluted with 16 μl of Tris-Tricine sample buffer. These aliquots were electrophoresed at 4° C. in 16.5% Tris-Tricine ready gel using Tris-Tricine-SDS electrode buffer at 100 volts (Mini Protean II, Biorad). The degree of proteolysis was analysed on gels fixed in 40% methanol, 10% acetic acid and stained with Coomassie blue.

Evaluation by Matrix Assisted Laser Desorption Ionization Mass Spectrometry (MALDI-MS)

In order to investigate the molecular weights of the metabolites, MS analysis was performed. Solutions in DI water containing 0.5 μM trypsin, 0.1 μM α-chymotrypsin and a mixture containing 0.5 μM trypsin as well as 0.1 μM α-chymotrypsin were incubated at 37° C. with 50 μM sCT for 0.5 min, 15 min and 0.5 min, respectively. After the above specified times the solutions were immediately diluted with cold 1% TFA and analysed by MS analysis. For this analysis, 1 μl of sample was mixed with 1 μl of the MALDI-TOF (Time Of Flight) matrix on a gold plated plate. The plates were allowed to dry and then it was inserted in the Maldi-TOF Voyager DE linear (Applied Biosystems, Foster City, Calif.) for analysis. The matrix consists of a saturated solution of α-cyano-4-hydroxycinnaminic acid (97%, F. W. of 189.17) with 50% acetonitrile and 0.1% TFA. The measurements were made in the positive ion mode. The ionizing and desorbing system consisted of a pulsed N₂-laser with appropriate UV-optics.

HPLC Analytical Method

sCT was analyzed by means of our validated HPLC method (Shah R B, et al., 2003). Briefly, a computer controlled Varian Chromatography workstation consisting of the following components was used: Two Dynamax SD-200 pumps, an AI-200A autosampler fitted with a 100 μl injection loop, a Dynamax UV-1 detector and Star 5.3 chromatography software. Room temperature was maintained for the column and chromatographic separations were carried out on a C-18 Vydac 218MS54 column (5 μm, 4.6×250 mm) with a pore size of 300 Å. 50 μl of samples were injected into the column which were analyzed by the reversed phase HPLC method. The mobile phase consisted of 0.05% v/v TFA-Water (A) and 0.05% v/v TFA-Acetonitrile (B). The gradient conditions were 20-35% B for 10 min, 35-37% B from 10^{th} to 20^{th} min and 37-20% B from 20^{th} to 25^{th} min at a flow rate of 1 ml/min. The detection was achieved at a wavelength of 210 nm. Concentrations of sCT were quantified from integrated peak areas and calculated by interpolation from an according standard curve.

Statistical Data Analysis

Statistical data analysis was performed using the student t test and ANOVA with P<0.05 as the minimal level of significance.

Results

Effects of Different Protease Inhibitors on Trypsin Mediated sCT Degradation

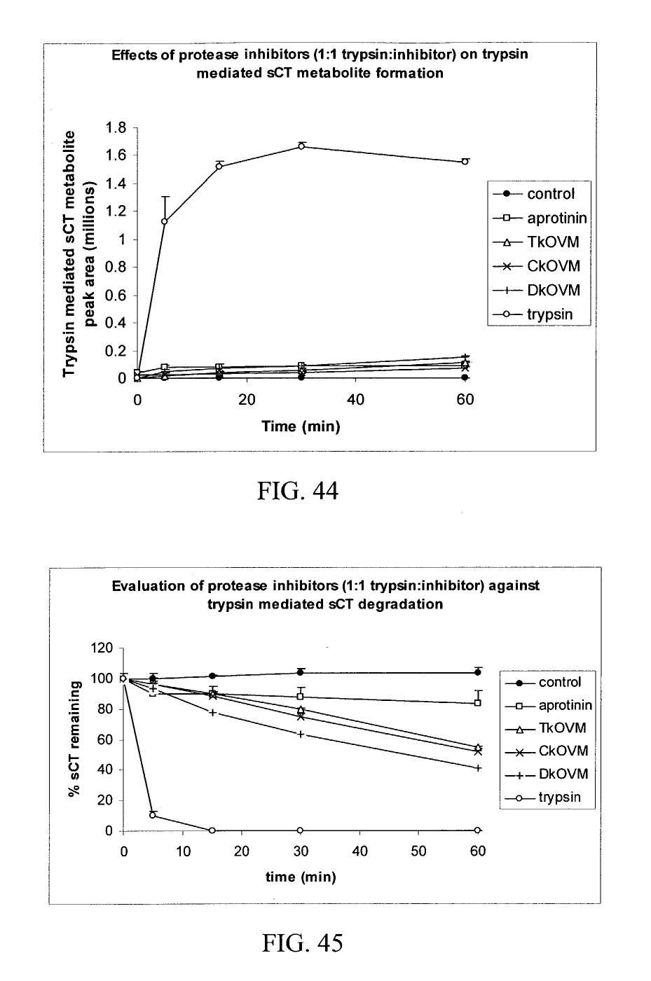

No significant changes in sCT concentration were observed in Tris-HCl buffer up to 2 hrs at 37° C. FIG. 44 shows the degradation profiles of sCT in the absence and presence of trypsin and protease inhibitors at 1:1 trypsin:inhibitor. The disappearance of sCT followed first-order kinetics. Table 23 summarizes the half-life of sCT in the presence of trypsin and different protease inhibitors at 1:1 trypsin:inhibitor ratio. When sCT alone was added to a buffer containing trypsin, rapid degradation was observed in the absence of protease inhibitors. However, aprotinin, chicken, duck and turkey ovomucoids effectively reduced the degradation of sCT in the presence of trypsin. Of the protease inhibitors investigated, the rank order of effectiveness for the protection of sCT against trypsin mediated degradation was aprotinin>TkOVM=CkOVM>DkOVM. Thus maximum reduction in proteolytic cleavage of sCT was seen in the presence of aprotinin whereas ovomucoids moderately inhibited the degradation of sCT. The metabolite 1 of sCT with retention time of 12.6 min was obtained by trypsin-mediated degradation. Effect of different protease inhibitiors at the same 1:1 trypsin:inhibitor ratio is shown in FIG. 45. As in accordance with the sCT protection, the metabolite 1 formed by trypsin-mediated degradation was maximum with trypsin, which decreased to a significant extent when the inhibitors were added in a buffer containing sCT and trypsin.

TABLE 23
Effects of protease inhibitors (1:1) on the half-life
of hydrolysis of sCT by trypsin

| | Half-life (min) | Ratio |
|---|---|---|
| Trypsin | 1.51 | 1.00 |
| Aprotinin (1:1) | 301 | 199.34 |
| CkOVM (1:1) | 62.4 | 41.32 |
| DkOVM (1:1) | 46.2 | 30.60 |
| TkOVM (1:1) | 69.3 | 45.89 |

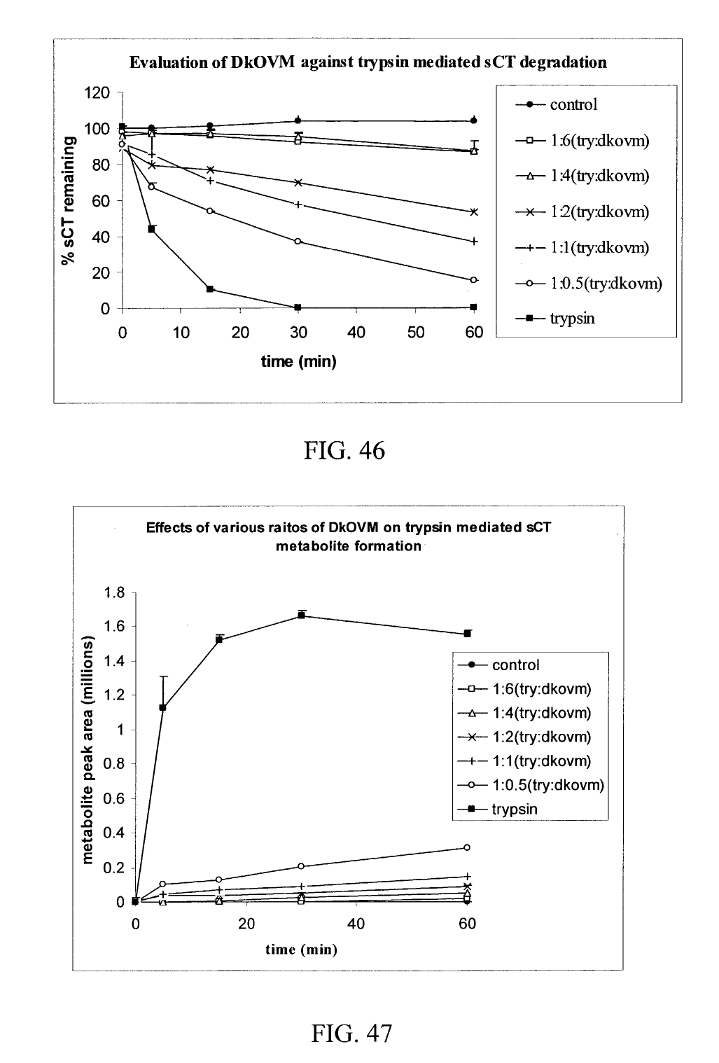

The degradation profiles of sCT in the presence of trypsin and different concentrations of DkOVM are shown in FIG. 46. The extent of degradation was decreased as the enzyme-to-inhibitor ratio was increased. At an enzyme-to-inhibitor ratio of 1:4 and 1:6, I₆₀ were 87.44+0.60 and 86.68±6.21, respectively. Table 24 summarizes the half-life of sCT in the presence of trypsin and DkOVM at various concentrations. The metabolite formation was decreased in the presence of DkOVM as seen in FIG. 47.

TABLE 24
Effects of DkOVM at various concentration on the half-life
of hydrolysis of sCT by trypsin

| | Half-life (min) | Ratio |
|---|---|---|
| Trypsin | 1.51 | 1.00 |
| DkOVM (1:0.5) | 24.14 | 15.99 |
| DkOVM (1:1) | 46.2 | 30.60 |
| DkOVM (1:2) | 88.84 | 58.83 |
| DkOVM (1:4) | 407 | 269.54 |
| DkOVM (1:6) | 330 | 218.54 |

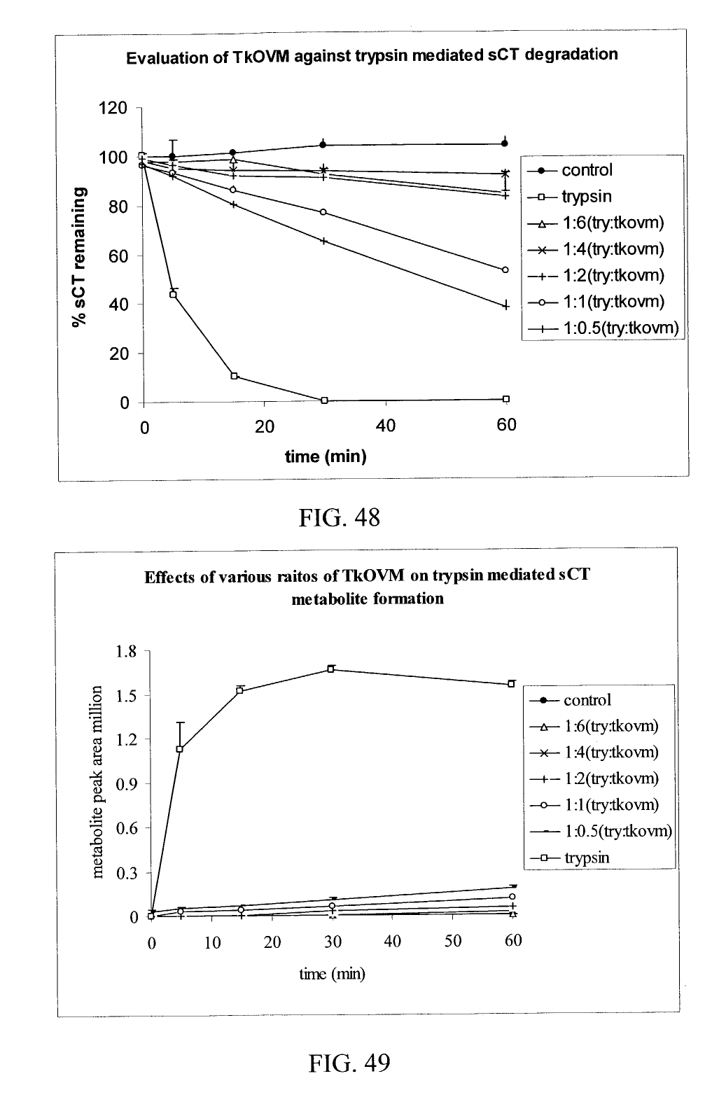

The degradation profiles of sCT in the presence of trypsin and different concentrations of TkOVM are shown in FIG. 48. The extent of degradation was decreased as the enzyme-to-inhibitor ratio was increased. At an enzyme-to-inhibitor ratio of 1:4 and 1:6, I₆₀ were 84.33±9.35 and 91.78±1.06, respectively. Table 25 summarizes the half-life of sCT in the presence of trypsin and DkOVM at various concentrations. The metabolite formation was decreased in the presence of TkOVM as seen in FIG. 49.

TABLE 25
Effects of TkOVM at various concentration on the
half-life of hydrolysis of sCT by trypsin

| | Half-life (min) | Ratio |
|---|---|---|
| Trypsin | 1.51 | 1.00 |
| TkOVM (1:0.5) | 44.42 | 29.42 |
| TkOVM (1:1) | 69.3 | 45.89 |
| TkOVM (1:2) | 247.5 | 163.91 |
| TkOVM (1:4) | 238.9 | 158.21 |
| TkOVM (1:6) | 256 | 169.54 |

Effects of Different Protease Inhibitors on α-Chymotrypsin Mediated sCT Degradation

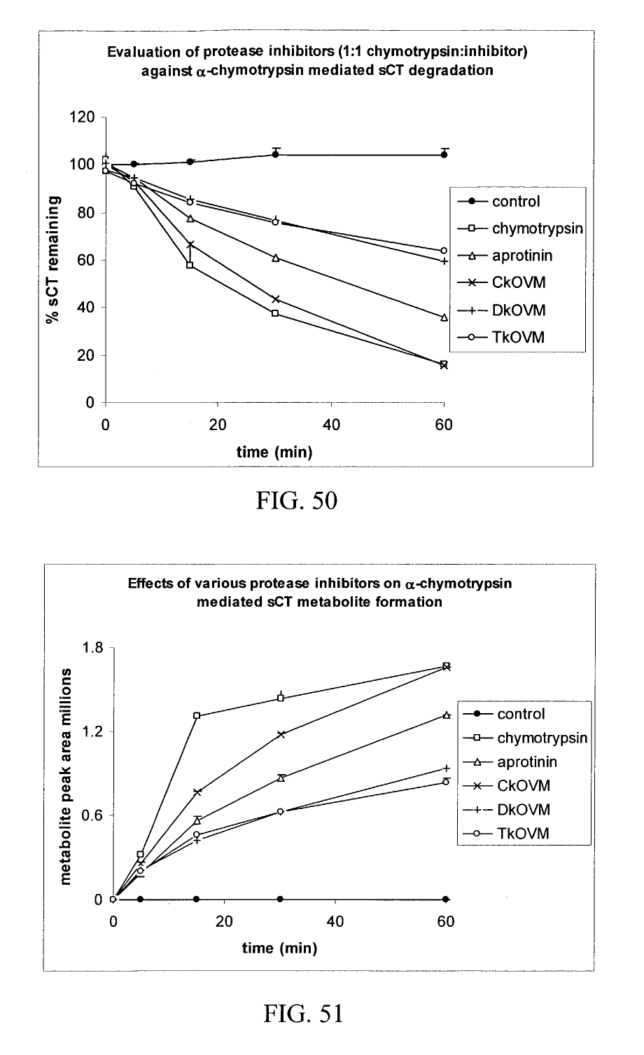

FIG. 50 shows the degradation profiles of sCT in the absence and presence of α-chymotrypsin and protease inhibitors at 1:1 trypsin:inhibitor. Table 26 summarizes the half-life of sCT in the presence of α-chymotrypsin and different protease inhibitors at 1:1 α-chymotrypsin:inhibition ratio. When sCT alone was added to a buffer containing α-chymotrypsin, significant degradation was observed. However, in the presence of different protease inhibitors significant reduction in the degradation was observed. Of the protease inhibitiors investigated, the rank order of effectiveness for the protection of sCT against trypsin mediated degradation was TkOVM>DkOVM>aprotinin. CkOVM was found to be ineffective in protecting sCT against α-chymotrypsin mediated degradation. The metabolite 2 of sCT with retention time of 13.3 min on HPLC was obtained by α-chymotrypsin-mediated degradation. Effect of different protease inhibitiors at the same 1:1 α-chymotrypsin:inhibitor ratio is shown in FIG. 51. As in accordance with the sCT protection, the metabolite 2 formed by α-chymotrypsin-mediated degradation was maximum with α-chymotrypsin, which decreased to a significant extent when the inhibitors were added in a buffer containing sCT and α-chymotrypsin.

TABLE 26
Effects of protease inhibitors (1:1) on the
half-life of hydrolysis of sCT by α-chymotrypsin

| | Half-life (min) | Ratio |
|---|---|---|
| α-chymotrypsin | 22.42 | 1.00 |
| Aprotinin (1:1) | 40.76 | 1.82 |
| CkOVM (1:1) | 22.07 | 0.98 |
| DkOVM (1:1) | 80.58 | 3.59 |
| TkOVM (1:1) | 99 | 4.42 |

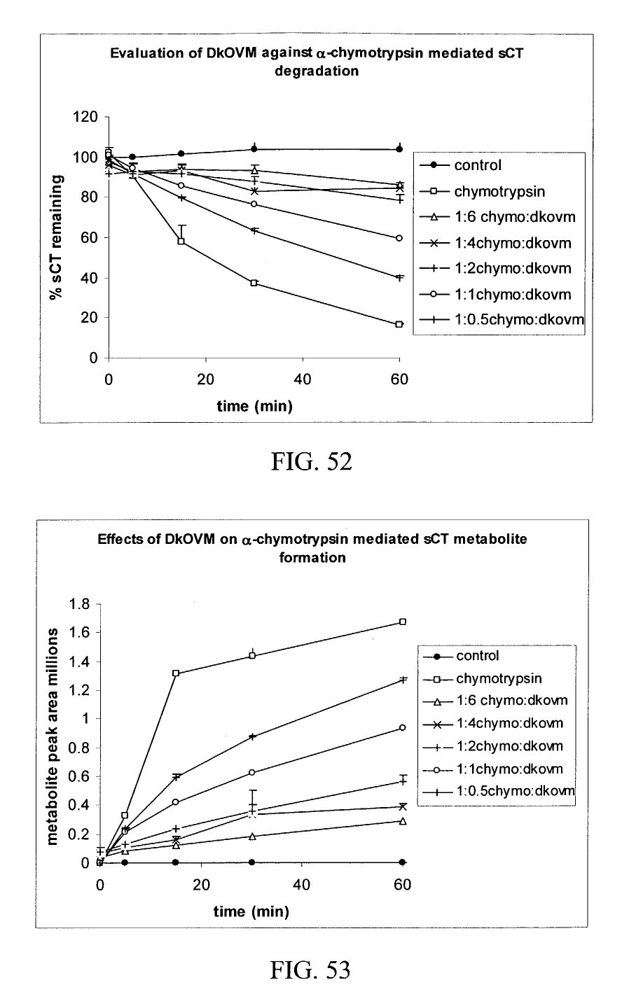

The degradation profiles of sCT in the presence of α-chymotrypsin and different concentrations of DkOVM are shown in FIG. 52. The extent of degradation was decreased as the enzyme-to-inhibitor ratio was increased. At an enzyme-to-inhibitor ratio of 1:4 and 1:6, I₆₀ values are given in Table 27. Table 28 summarizes the half-life of sCT in the presence of α-chymotrypsin and DkOVM at various concentrations. The metabolite formation was decreased in the presence of DkOVM as seen in FIG. 53.

TABLE 28
Effects of DkOVM at various concentration on the
half-life of hydrolysis of sCT by α-chymotrypsin

| | Half-life (min) | Ratio |
|---|---|---|
| α-chymotrypsin | 22.42 | 1.00 |
| DkOVM (1:0.5) | 45.89 | 2.05 |
| DkOVM (1:1) | 80.58 | 3.59 |
| DkOVM (1:2) | 256.6 | 11.45 |
| DkOVM (1:4) | 346.5 | 15.45 |
| DkOVM (1:6) | 407.6 | 18.18 |

TABLE 27
Amount (%) of sCT remaining at 60 min (I₆₀)
in the presence of proteases, Caco-2 cell and goat
intestinal homogenate with or without protease inhibitors.

| Combination with sCT | I₆₀ |
|---|---|
| control | 103.76 ± 3.10 |
| trypsin (0.5 μM) | 0.00 ± 0.00 |
| trypsin + aprotinin 1:1 | 83.36 ± 8.92 |
| trypsin + CkOVM 1:1 | 51.79 ± 2.44 |
| trypsin + DkOVM 1:1 | 40.78 ± 2.56 |
| trypsin + TkOVM 1:1 | 54.77 ± 0.42 |
| trypsin + DkOVM 1:0.5 | 14.99 ± 0.66 |
| trypsin + DkOVM 1:1 | 37.21 ± 2.34 |
| trypsin + DkOVM 1:2 | 53.63 ± 1.73 |
| trypsin + DkOVM 1:4 | 87.44 ± 0.60 |
| trypsin + DkOVM 1:6 | 86.68 ± 6.21 |
| trypsin + TkOVM 1:0.5 | 38.09 ± 2.68 |
| trypsin + TkOVM 1:1 | 52.75 ± 0.41 |
| trypsin + TkOVM 1:2 | 82.87 ± 4.27 |
| trypsin + TkOVM 1:4 | 84.33 ± 9.35 |
| trypsin + TkOVM 1:6 | 91.78 ± 1.06 |
| chymotrypsin (0.1 μM) | 16.18 ± 0.48 |
| chymotrypsin + aprotinin 1:1 | 35.87 ± 1.06 |
| chymotrypsin + CkOVM 1:1 | 15.58 ± 0.15 |
| chymotrypsin + DkOVM 1:1 | 59.59 ± 1.04 |
| chymotrypsin + TkOVM 1:1 | 63.67 ± 1.37 |
| chymotrypsin + DkOVM 1:0.5 | 40.00 ± 0.92 |
| chymotrypsin + DkOVM 1:1 | 59.59 ± 1.04 |
| chymotrypsin + DkOVM 1:2 | 78.78 ± 2.63 |
| chymotrypsin + DkOVM 1:4 | 84.64 ± 2.39 |
| chymotrypsin + DkOVM 1:6 | 85.96 ± 1.42 |
| chymotrypsin + TkOVM 1:0.5 | 43.66 ± 0.67 |
| chymotrypsin + TkOVM 1:1 | 63.67 ± 1.37 |
| chymotrypsin + TkOVM 1:2 | 81.21 ± 2.73 |
| chymotrypsin + TkOVM 1:4 | 91.62 ± 1.52 |
| chymotrypsin + TkOVM 1:6 | 90.93 ± 1.41 |
| trypsin (0.5 μM) + chymotrypsin (0.1 μM) | 0.00 ± 0.00 |
| trypsin + chymotrypsin + TkOVM (1:4) | 54.25 ± 3.14 |
| trypsin + chymotrypsin + TkOVM (1:6) | 86.92 ± 2.19 |
| trypsin + chymotrypsin + TkOVM (1:8) | 96.27 ± 3.75 |
| Caco-2 homogenate | 81.46 ± 10.89 |
| Goat intestinal homogenate | 91.69 ± 17.60 |

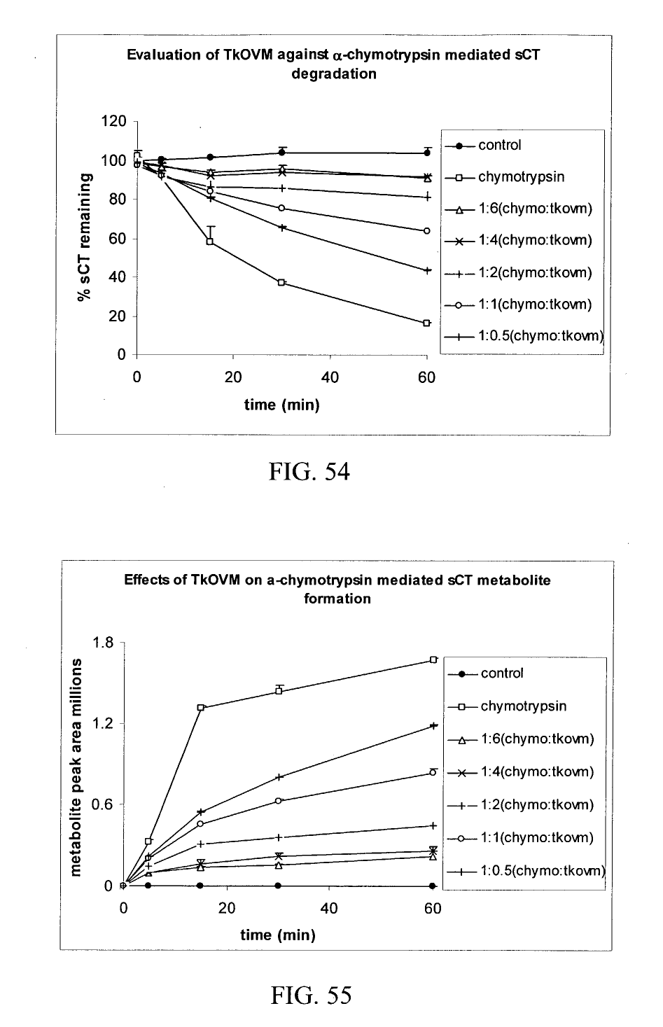

The degradation profiles of sCT in the presence of α-chymotrypsin and different concentrations of TkOVM are shown in FIG. 54. The extent of degradation was decreased as the enzyme-to-inhibitor ratio was increased. Table 29 summarizes the half-life of sCT in the presence of α-chymotrypsin and DkOVM at various concentrations. The metabolite formation was decreased in the presence of TkOVM as seen in FIG. 55.

TABLE 29
Effects of TkOVM at various concentration on the
half-life of hydrolysis of sCT by α-chymotrypsin

| | Half-life (min) | Ratio |
|---|---|---|
| α-chymotrypsin | 22.42 | 1.00 |
| TkOVM (1:0.5) | 50.58 | 2.26 |
| TkOVM (1:1) | 99 | 4.42 |
| TkOVM (1:2) | 247.5 | 11.04 |
| TkOVM (1:4) | 577.5 | 25.76 |
| TkOVM (1:6) | 577.5 | 25.76 |

Effects of TkOVM on Trypsin and α-Chymotrypsin Mediated sCT Degradation

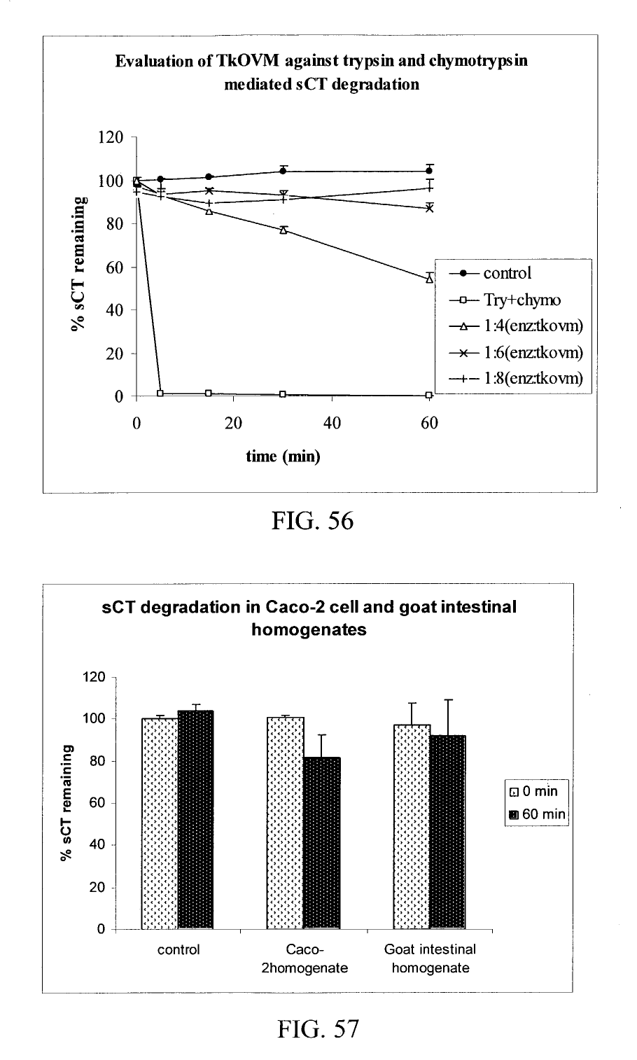

When sCT was added to both the proteases together, there was a rapid decrease in amount of sCT and all of it degraded within 5 minutes. TkOVM added to protected sCT from the proteases depending on the concentration added. 1:4 ratio with respect to trypsin and α-chymotrypsin was 2.4 μM, but it was not found to be 100% protective to sCT. The TkOVM ratio of 1:8 was found to be the best protective for sCT. FIG. 56 depicts the sCT metabolism by both, trypsin and chymotrypsin added together and protection by TkOVM. Table 30 summarizes the half-life and ratio of sCT in both the proteases with or without TkOVM.

TABLE 30
Effects of TkOVM at various concentration on the
half-life of hydrolysis of sCT by trypsin and α-chymotrypsin

| | Half-life (min) | Ratio |
|---|---|---|
| Trypsin and α-chymotrypsin | 0.77 | 1.00 |
| TkOVM (1:4) | 70 | 90.91 |
| TkOVM (1:6) | 433.12 | 562.49 |
| TkOVM (1:8) | 17325 | 22500.00 |

In Vitro Metabolism of sCT in Caco-2 Cell and Goat Intestinal Homogenates

The degradation of sCT in homogenates of FIG. 57 shows the metabolism of sCT in the homogenates of the Caco-2 and goat intestine. The trypsin and chymotrypsin contents are given in Table 31. In accordance with the protease contents, Caco-2 metabolised sCT in 60 min to a higher extent than intestinal homogenate. As the intestinal tissue was washed thrice with PBS, all luminal contents were washed off; therefore, the luminal enzyme activity was minimum as the fluids of the small intestine was removed prior to homogenization. The protease activity was higher in Caco-2 as compared to the intestinal homogenate as seen in FIG. 57.

TABLE 31
Trypsin and chymotrypsin contents of
Caco-2 cell and goat intestinal homogenates

| | Caco-2 cell | Goat intestinal |
|---|---|---|
| | homogenate | homogenate |
| Protein content (mg/ml) | 1.3 | 31.1 |
| Trypsin (μM) | 0.22 | 0.00056 |
| α-Chymotrypsin (μM) | 0.029 | 0.0045 |
| OR | | |
| Trypsin (μmoles/mg) | 0.169 | 0.000018 |
| α-Chymotrypsin | 0.0223 | 0.000144 |
| (μmoles/mg) | | |

Evaluation of Metabolites of sCT

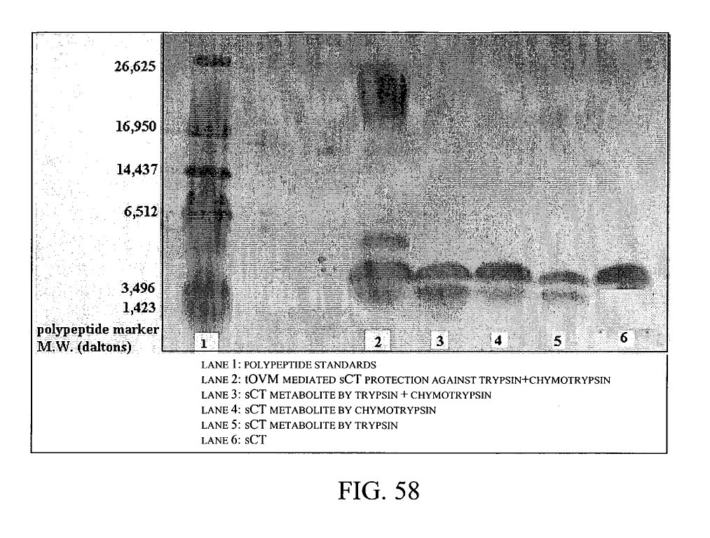

Gel electrophoresis of sCT samples with or without proteases are shown in FIG. 58. sCT showed a band at M.W. 3440 Da and there were no other bands when sCT was electrophoresed alone. But with trypsin and chymotrypsin incubation, the degradation of sCT was observed. As it is unlikely to get the same M.W. band with both trypsin and chymotrypsin further evaluation was undertaken.

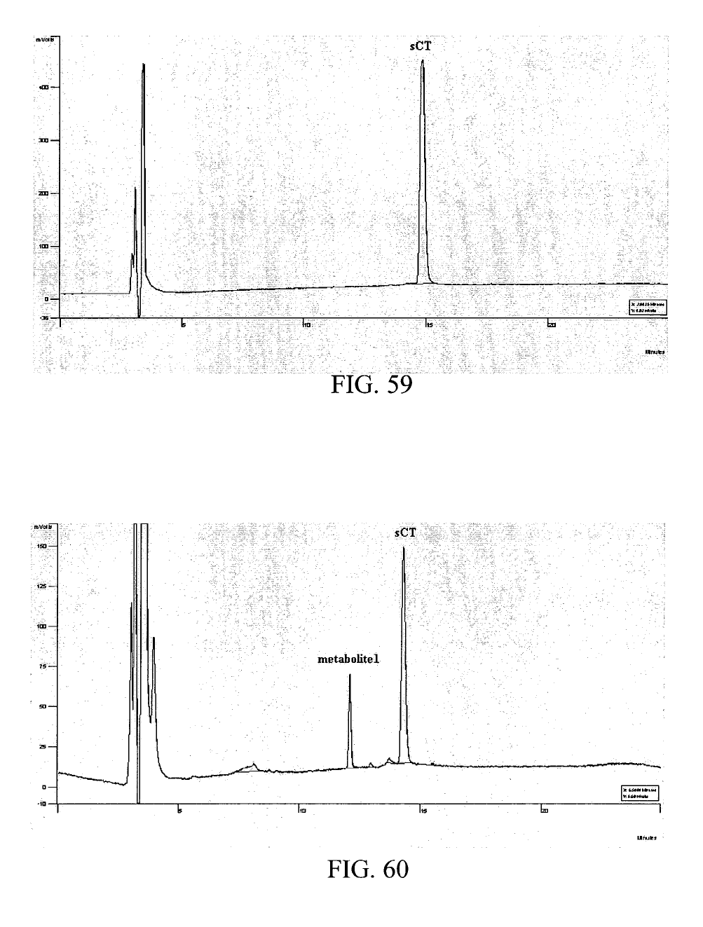

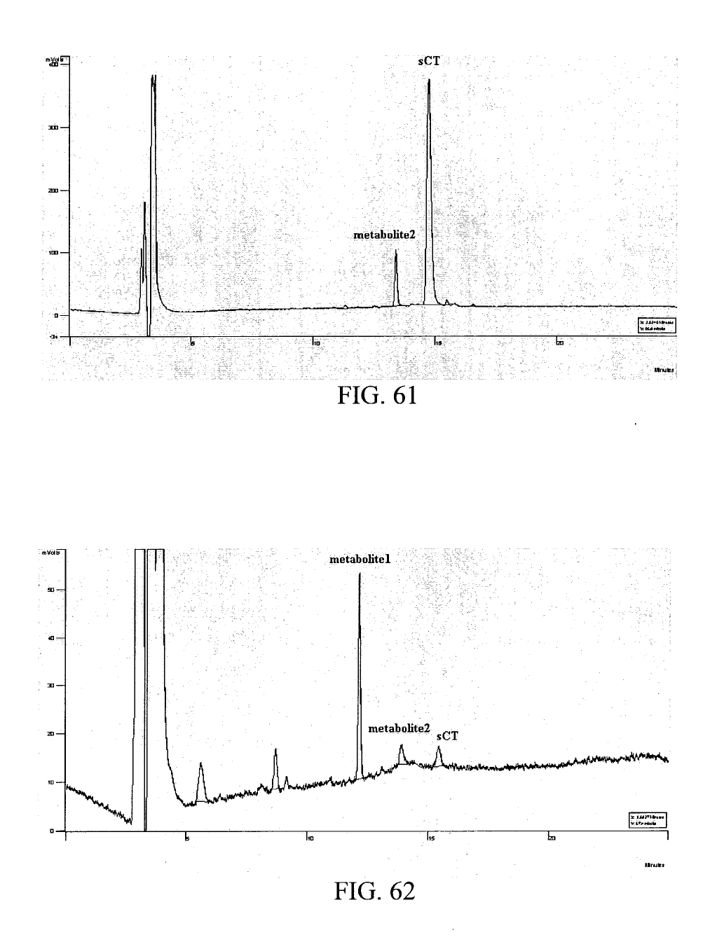

An HPLC chromatogram of sCT (FIG. 59), HPLC chromatogram of trypsin mediated sCT metabolite (FIG. 60), HPLC chromatogram of chymotrypsin mediated sCT metabolite (FIG. 61), and HPLC chromatogram of sCT metabolites formed by trypsin and chymotrypsin (FIG. 62) are shown. There were no other peaks other than sCT at 14 min when it was incubated alone without any proteases. However, a metabolite 1 peak at 12.6 min and metabolite 2 peak at 13.3 min was obtained with trypsin and chymotrypsin, respectively. When sCT was incubated with trypsin and chymotrypsin, the two metabolites 1 and 2 were obtained at the respective retention time.

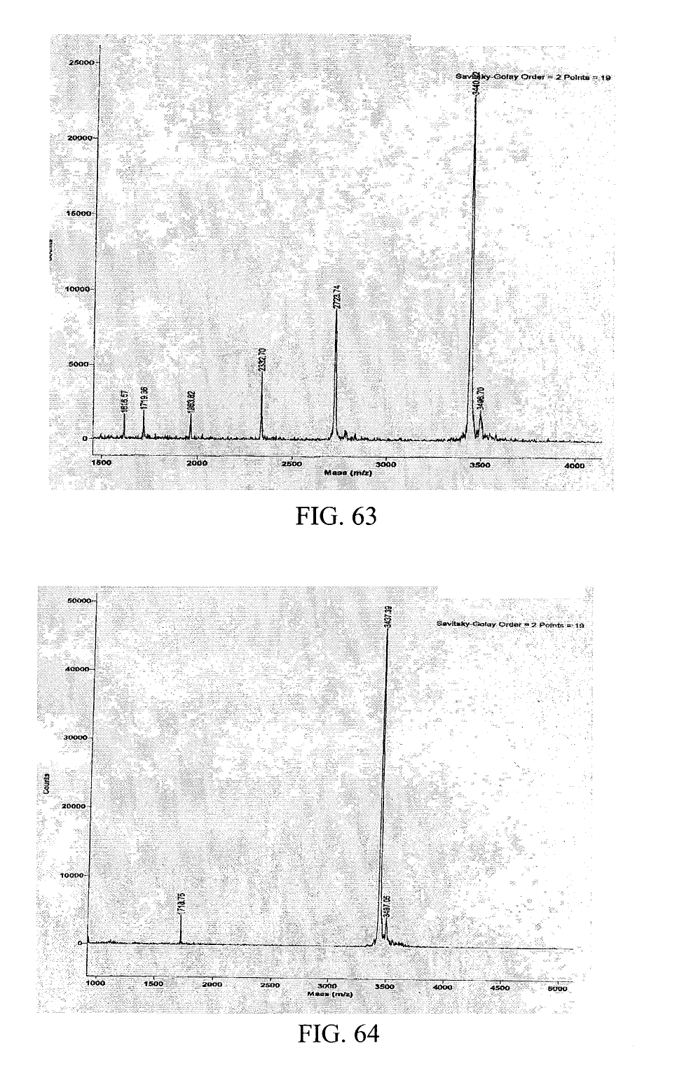

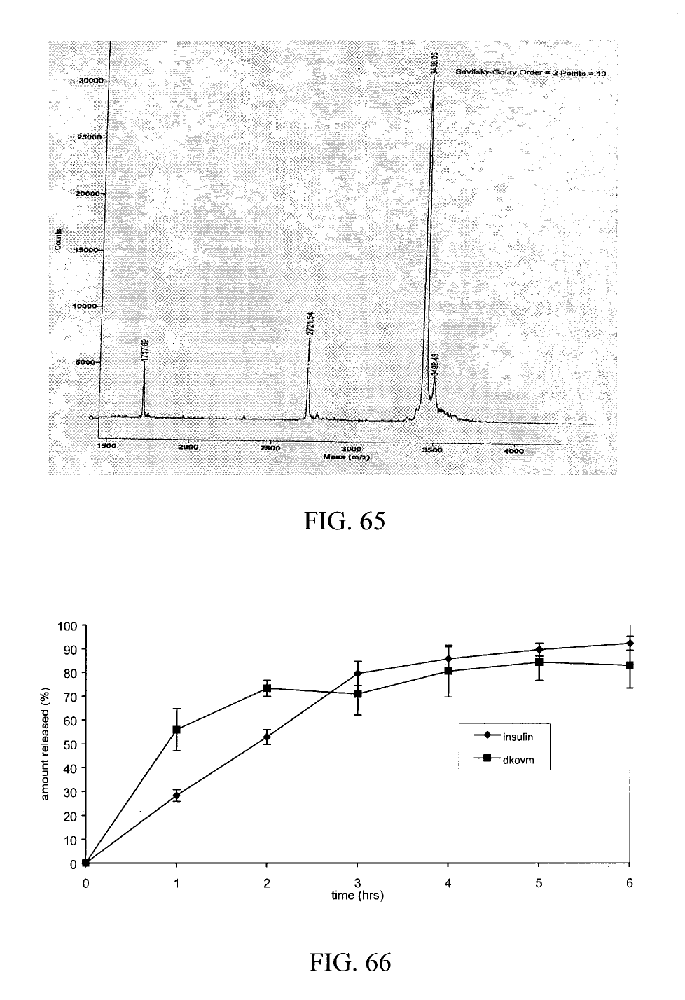

MS spectra are shown in FIG. 63, FIG. 64, and FIG. 65. FIG. 63 shows an MS chromatogram of trypsin mediated sCT degradation and metabolite formation. FIG. 64 shows an MS chromatogram of chymotrypsin mediated sCT degradation and metabolite formation. FIG. 65 shows an MS chromatogram of trypsin and chymotrypsin mediated sCT degradation and metabolite formation. Incubation with trypsin showed many peaks as compared to chymotrypsin. As it was expected as the structure of the peptide revealed only one cleavage site for chymotrypsin compared to 4 cleavage sites for trypsin. The fragments of different M.W. were obtained as shown in Table 32.

TABLE 32
MALDI-MS peak spectrum of metabolites (M + H)⁺ of sCT.

| | Peak No. | MALDI | Fragments |
|---|---|---|---|
| Trypsin | 1 | 1615.57 | 18-23 |
| | 2 | 1719.36 | 11-17 |
| | 3 | 1963.82 | 24-32 |
| | 4 | 2332.70 | |
| | 5 | 2723.74 | 1-23 |
| | 6 | 3440.02 | 1-32 (intact sCT) |
| α-Chymotrypsin | 1 | 1718.75 | 22-32 |
| | 2 | 3437.39 | 1-32 (intact sCT) |
| Trypsin + α-chymotrypsin | 1 | 1717.69 | 22-32 |
| | 2 | 2721.55 | |
| | 3 | 3438.03 | 1-32 (intact sCT) |

Discussion

Within this study, novel excipients for the peroral administration of calcitonin, providing a strong protective effect towards enzymatic attack of intestinal proteases have been generated. Among these enzymes, luminally secreted serine proteases seem to be mainly responsible for digestion of salmon calcitonin (Sakuma et al. 1997), (Sakuma et al. 1997), for instance, could demonstrate a high cleavage rate of calcitonin caused by trypsin. Furthermore, Dohi et al. (1993), and Lang et al. (1996), showed that the peptide is digested not only by trypsin but also by α-chymotrypsin. Elastase is also reported to degrade salmon calcitonin recently. (Guggi and Bernkop-Schnurch, 2003). The presystemic metabolism of perorally administered calcitonin caused by luminally secreted proteases should be strongly reduced.

Ovomucoid is a glycoprotein with molecular weight between 20 kDa to 30 kDa. Protein component of ovomucoid molecules can interact with serine proteolytic enzymes and inhibit their activities (Plate et al., 1993; Plate et al. 1993). At the same time polysaccharide component of ovomucoid can form a complex with lectins (proteins which recognize and bind sugars in glycoconjugates) (Woodley J F, 2000 Woodley J F. 2000). DkOVM was investigated as discussed above to successfully deliver insulin orally. Also a new approach to overcome the degradation of protein drugs by proteolytic enzymes and their targeting to the blood through the digestive apparatus was developed (Plate et al., 2002; Plate et al. 2002). The approach is based on the immobilization of drugs into the polymeric hydrogel containing glycoprotein—ovomucoid from duck egg whites. This glycoprotein inhibits the activity of proteolytic enzymes and acts as a biospecific ligand to lectins on the walls of the gastrointestinal tract.

sCT is well known to be unstable in aqueous solvents at ambient temperature. sCT is reported to be more stable in solvents of acidic pH compared to neutral or alkaline pH (Lee et al. 1992). However, it was fairly stable for 1 hr at 37° C. based on the fact that the peak area of sCT obtained was unchanged under given conditions as shown as control in FIG. 44, FIG. 46, FIG. 48, and FIG. 50. TFA used to stop the proteolytic reaction is known to stabilize sCT. (Song et al. 2002). This indicates that sCT is stable enough during the assay procedures.

The degradation profiles of sCT in the presence of trypsin and different enzyme inhibitors at 1:1 enzyme:inhibitor ratio is shown in FIG. 44. The extent of degradation decreased in the presence of inhibitors. Among the inhibitiors studied, the rank order of effectiveness for the protection of sCT against trypsin mediated degradation was aprotinin>TkOVM=CkOVM>DkOVM. Thus maximum reduction in proteolytic cleavage of sCT was seen in the presence of aprotinin whereas ovomucoids moderately inhibited the degradation of sCT. TkOVM and CkOVM were found to be more effective in protecting sCT against trypsin mediated degradation as compared with DkOVM. DkOVM was previously shown, as described above, to be the most protective for insulin against trypsin mediated degradation as compared to CkOVM. However, our studies in this experimentation with sCT did not show the similar trend. This might be due to the fact that the purity of DkOVM cannot be guaranteed as it is not available commercially. Therefore, at the same micromolar concentration, DkOVM might have some impurities and it is not effective to the same extent. When increasing concentration of DkOVM was used, at 1:4 or 1:6 ratios, the degradation of sCT was minimum (FIG. 46). When TkOVM was added in increasing concentration, the degradation of sCT was negligible even at 1:2 ratio (FIG. 48).

When comparing different enzyme inhibitors for protecting sCT against α-chymotrypsin, it was seen that DkOVM as well as TkOVM were both equally effective. The extent of degradation by α-chymotrypsin was not affected by the presence of CkOVM. Also, as described above, it was shown for insulin degradation by α-chymotrypsin was not affected by CkOVM even at 1:4 enzyme:inhibitor ratio. It is clear that the inhibitory action of ovomucoids is enzyme- and species-dependent. Ovomucoids belong to the pancreatic secretory trypsin family of inhibitors (Laskowski and Kato, 1960). Each inhibitor molecule has at least one peptide bond, known as the reactive site, that interacts with the corresponding enzyme by means of Van der Waals interaction, hydrogen-bonding and salt bridges. CkOVM has only one inhibitory site for trypsin whereas DkOVM and TkOVM has two sites for trypsin and one each for chymotrypsin, subtilin and elastase. The results presented show the inhibitory action of TkOVM and DkOVM when sCT is used as substrate. Further inhibitory response curves were established as a function in the range 0.25 to 3.0 μM for trypsin and 0.05 to 0.6 μM for chymotrypsin for the TkOVM and DkOVM studied. Aprotinin is a non-specific protease inhibitor derived from bovine lung tissue and is associated with anti-fibrinolytic activity and preservation of platelet function (Robert et al., 1996). If it is administered orally it undergoes gastric inactivation (Royston, 1992).

Evaluation of Metabolites of sCT Produced by Trypsin and α-Chymotrypsin

The identified metabolites of sCT by trypsin and α-chymotrypsin are shown in HPLC chromatograms (FIG. 61 and FIG. 62). Comparing the metabolites produced at 0.5 min after incubating with trypsin and 15 min after incubating with α-chymotrypsin and 0.5 min after incubating with trypsin and α-chymotrypsin with that of sCT chromatogram (FIG. 60), trypsin produced a major metabolite with retention time of 12.8 min. The peak areas of metabolite I produced by trypsin and metabolite II produced by α-chymotrypsin were monitored in the presence and absence of enzyme inhibitors. The formation of metabolites 1 and 2 were absent in the presence of DkOVM and TkOVM (FIG. 45, FIG. 47, FIG. 49, FIG. 51, and FIG. 53). Other metabolites were not detected by HPLC within 1 hour. The potential cleavage sites for trypsin, α-chymotrypsin and elastase present in the structure of sCT was shown in one of the studies (Guggi and Bernkop-Schnurch, 2003).

MALDI-MS of sCT Metabolites

MALDI-MS analyses were performed directly from the sCT incubation solution. The resulting spectra contained signals of the (M+H)⁺ ion of each peptide present in the incubation solution. The results of the MALDI-MS of sCT incubation solution is shown in FIG. 63, FIG. 64, and FIG. 65. The incubation times were 0.5 min, 15 min and 0.5 min for tryptic, chymotryptic and both, tryptic and chymotryptic digests, respectively.

Matrix-assisted laser desorption/ionisation-time of flight mass spectrometry (MALDI-TOF MS) is a relatively novel technique in which a co-precipitate of an UV-light absorbing matrix and a biomolecule is irradiated by a nanosecond laser pulse. Most of the laser energy is absorbed by the matrix, which prevents unwanted fragmentation of the biomolecule. The ionized biomolecules are accelerated in an electric field and enter the flight tube. During the flight in this tube, different molecules are separated according to their mass to charge ratio and reach the detector at different times. In this way each molecule yields a distinct signal.

In Vitro Metabolism of sCT in Caco-2 Cell and Goat Intestinal Homogenates

The proteolytic activity of luminal extracts from small intestine towards sCT was investigated. There were no significant differences in the sCT concentration remaining after incubating with goat intestinal homogenate as compared to control without any enzyme. But a significant proteolysis was observed with Caco-2 cell homogenate as compared to control. This finding suggests that serine proteases such as trypsin and chymotrypsin are responsible for degradation of sCT in the homogenate of Caco-2 cell.

### EXAMPLE 7

### Design and In Vivo Evaluation of a Novel Oral Drug Delivery System for Proteins with Ovomucoid

Oral delivery of proteins has limitations with respect to enzymatic degradation in the GIT, poor absorption and formulation stability issues. Some approaches to overcome these limitations are use of enzymatic inhibitors, absorption modifiers and polymeric preparations of proteins. A synergistic approach of polymeric preparation with enzyme inhibitors or/and permeation enhancers has shown promising results. However, the toxicity due to high inhibitor concentrations remains a challenge for oral delivery.

Ovomucoids are enzyme inhibitors isolated from the egg white of avian species. They have two properties that are attractive 1) inhibitory action that is dependent upon the species from which they are isolated and 2) they have a tendency to bind to lectins through their carbohydrate moiety for enhanced bioadhesion. Their role in the oral delivery of proteins has not been investigated in detail. In the present study, a representative inhibitor, duck ovomucoid (DkOVM) has been incorporated in the dosage form and tested for improvement in in vivo efficacy.

Purpose

One of the approaches to reduce the toxicity may be to control the release of inhibitor with the active substance. This will have a two-fold advantage over immediate release of inhibitor-exposure of GIT to small amounts and extended inhibitory action. The purpose of the present investigation was to develop a dosage form that incorporates dual controlled release characteristics of protein and ovomucoid. To evaluate the in vivo efficacy of dosage form, relative hypoglycemia (R.H.) values were assessed.

Methods

Preparation of Dosage Form Containing Insulin and DkOVM

Insulin microcapsules were prepared by a coprecipitation technique. Briefly, insulin was dissolved in a mixture of ethyl alcohol and 0.01N HCl. This was transferred using a peristaltic pump to a beaker containing cold under vigorous stirring with a homogenizer. Microcapsules were separated by filtration and dried in an oven.

To optimize the critical process variables that affect the dissolution of microcapsules, a three-factor three-level optimization design was used. The variables tested were rate of addition of polymeric drug solution with respect to precipitating medium, volume of precipitating medium and compression pressure. The formulation had insulin microcapsules, DkOVM, talc, lactose and magnesium stearate. The tablet ingredients were compressed using an automated carver press assembly. Dissolution was performed for 6 hours at 37 C in pH 6.8 phosphate buffer. The dissolution assembly was fitted with a conversion kit for 100 ml.

HPLC Analysis

Analysis was performed using a novel HPLC method that allowed for simultaneous analysis of insulin and duck ovomucoid. The mobile phase consisted of 0.05% v/v TFA/Water (A) and 0.05% v/v TFA/Acetonitrile (B). The mobile phase conditions were 20% B for 5 mins, 20-52% B for the next 15 mins, 52%-20% B in the next 10 mins. The flow rate was set at 1 ml/min and the detection wavelength was 210 nm. The column used was a Vydac 218MS54 C₁₈ column (4.6×30 mm)

In Vivo Studies

In vivo studies were performed in 6 rabbits using a three-way crossover design with two replicates. The oral formulations tested were tablet without inhibitor (insulin 14 IU/kg) and tablet with inhibitor (insulin 14 IU/kg and DkOVM 2.7 mg/kg). Subcutaneous injection (Novolin®, 1.5 IU/kg) served as the control. A plot of glucose reduction (%) versus time was constructed and relative hypoglycemia values were calculated from the AUC.

Results and Discussion

The role of ovomucoids is being investigated in our laboratory for enhanced oral delivery of insulin. In the presence of ovomucoids, we have seen improved enzymatic stability, enhanced flux in rat jejunum, and increased dissolution stability of microcapsules in the presence of enzymes.

In the present investigation we report the development of oral dosage form with ovomucoid and testing of its in vivo efficacy. Response surface methodology was used in the optimization studies to provide optimum levels of response −100% release in 6 hours with Higuchi's square root of time dissolution kinetic model. The optimized values of the independent variables predicted were rate of addition 20 ml/min, volume of water 80.7 ml and compression pressure 1.2 tons. Dissolution profiles generated were in close agreement with predicted values. The release of DkOVM from the optimized formulation also followed Higuchi's square root of time kinetics over 6 hours. The dissolution profile of insulin and DkOVM form the optimized formulation is shown in FIG. 66.

In the HPLC analysis, baseline separation was achieved. Retention time of DkOVM was 11.7 min and that of insulin was 14.5 min.

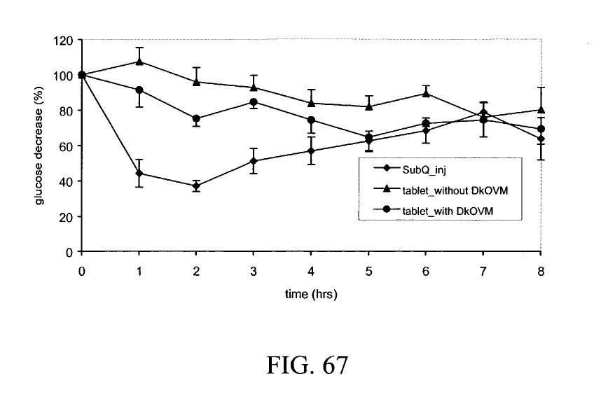

The hypoglycemic effect of the various formulations is shown in FIG. 67. In vivo efficacies of oral formulations were significantly different (p<0.01) than Sub Q injection. Formulation with inhibitor was significantly different (p<0.05) from formulation without inhibitor. The relative hypoglycemia of the formulations calculated after adjustment of dose was 1.6% for the formulation without inhibitor and 5.0% for formulation with inhibitor.

Conclusions

In the present study enhanced delivery of insulin is reported in the presence of DkOVM. Optimized dual controlled release tablet formulation containing microcapsules of insulin and DkOVM displayed a 3.2 fold greater hypoglycemic effect when compared to a similar preparation without ovomucoid. The hypoglycemic activity was due to inhibition of luminal enzymes, enhanced flux of insulin and increased dissolution stability.

Selection of proper concentration of inhibitor may address the issue of systemic intoxication of inhibitors in vivo. The toxicity of inhibitor may be further reduced by modification of the dosage form to release it slowly.

Equivalents

Those skilled in the art will be able to recognize, or be able to ascertain using no more than routine experimentation, many equivalents to the specific embodiments of the invention described herein. Such equivalents are intended to be encompassed by the following claims.

We claim:

1. A therapeutic composition adapted for oral administration, comprising:
an absorption modifier;
a polymeric carrier; and
a biologically active protein;
wherein said absorption modifier and said protein are controllably released within the body.

2. The composition of claim 1, wherein the controlled release of said protein occurs in the small intestine.

3. The composition of claim 1, wherein the absorption modifier is an enzyme inhibitor.

4. The composition of claim 1, wherein the absorption modifier is a permeation enhancer.

5. The composition of claim 1, wherein the absorption modifier is an enzyme inhibitor and a permeation enhancer.

6. The composition of claim 1, wherein the absorption modifier is an ovomucoid.

7. The composition of claim 6, wherein said ovomucoid is selected from the group consisting of chicken and duck ovomucoid.

8. The composition of claim 1, wherein said polymeric carrier is an aqueous methacrylic polymer having a pH sensitive solubility.

9. The composition of claim 8, wherein said polymeric carrier is an anionic polymer solubilizing above pH 5.0.

10. The composition of claim 8, wherein said polymeric carrier is an anionic polymer solubilizing above pH 6.0.

11. The composition of claim 8, wherein said polymeric carrier is an anionic polymer solubilizing above pH 7.0.

12. The composition of claim 1, wherein the protein is insulin.

13. The composition of claim 1, wherein the protein is calcitonin.

14. A method for microencapsulating a protein by a coprecipitation technique to form a polymeric system that maximizes the cumulative amount of protein released at the end of a targeted delivery time, comprising the steps of:
adding polymeric solution at a rate in the range of 10-20 ml/min., and applying compression pressure in the range of 0.6-1.2 tons,
to an aqueous solution having a volume of water to volume of polymeric solution in the range of 50-150.

15. The method of claim 14, wherein the protein is insulin.

16. The method of claim 14, further including the step of compressing an absorption modifier within the polymeric system such that the absorption modifier is gradually released.

17. The method of claim 14, further including the step of microencapsulating an absorption modifier such that the absorption modifier is gradually released.

18. The method of claim 14, wherein the stability and bio-availability of the protein is increased.

19. A method of orally administering one or more biologically active materials comprising the steps of:
preparing a composition for oral ingestion containing an absorption modifier; a polymeric carrier; and a biologically active protein; wherein said absorption modifier and said protein are controllably released within the body; and
orally administering said composition to a human or animal specie.

20. The method of claim 19, wherein the controlled release of said protein occurs in the small intestine.

21. The method of claim 19, wherein the absorption modifier is an enzyme inhibitor.

22. The method of claim 19, wherein the absorption modifier is a permeation enhancer.

23. The method of claim 19, wherein the absorption modifier is an enzyme inhibitor and a permeation enhancer.

24. The method of claim 19, wherein the absorption modifier is an ovomucoid.

25. The method of claim 24, wherein said ovomucoid is selected from the group consisting of chicken and duck ovomucoid.

26. The method of claim 19, wherein said polymeric carrier is an aqueous methacrylic polymer having a pH sensitive solubility.

27. The method of claim 26, wherein said polymeric carrier is an anionic polymer solubilizing above pH 5.0.

28. The method of claim 26, wherein said polymeric carrier is an anionic polymer solubilizing above pH 6.0.

29. The method of claim 26, wherein said polymeric carrier is an anionic polymer solubilizing above pH 7.0.

30. The method of claim 19, wherein the protein is insulin.

31. The method of claim 19, wherein the protein is calcitonin.
